SEQUENCE LISTING (129 sequences)

SEQ ID NO: 1 - protein, 15 aa, Artificial Sequence
  Description of Artificial Sequence Synthetic peptide motif
XXXHXXGXXXXXXXG

SEQ ID NO: 2 - protein, 32 aa, Artificial Sequence
  Description of Artificial Sequence Synthetic peptide motif
XXXXXXXXXXXXXXXXXXXXXXXXDXXXGGXX

SEQ ID NO: 3 - protein, 14 aa, Artificial Sequence
  Description of Artificial Sequence Synthetic peptide motif
DRSPXGXGXXAXXA

SEQ ID NO: 4 - protein, 14 aa, Artificial Sequence
  Description of Artificial Sequence Synthetic peptide motif
DRSPCGXGXXAXXA

SEQ ID NO: 5 - DNA, 28 bp, Artificial Sequence
  Description of Artificial Sequence Synthetic oligonucleotide
ctctcccatggggcaggaaaagcttctg

SEQ ID NO: 6 - DNA, 23 bp, Artificial Sequence
  Description of Artificial Sequence Synthetic oligonucleotide
ctgagctcgaccagatmtactgc

SEQ ID NO: 7 - DNA, 30 bp, Artificial Sequence
  Description of Artificial Sequence Synthetic oligonucleotide
gcggatcgctctccaagcgggacaggcacc

SEQ ID NO: 8 - DNA, 30 bp, Artificial Sequence
  Description of Artificial Sequence Synthetic oligonucleotide
ggtgcctgtcccgcttggagagcgatccgc

SEQ ID NO: 9 - DNA, 39 bp, Artificial Sequence
  Description of Artificial Sequence Synthetic oligonucleotide
ggctatttaaatatgtctggacataactcaattgcagcg

SEQ ID NO: 10 - DNA, 38 bp, Artificial Sequence
  Description of Artificial Sequence Synthetic oligonucleotide
cgctgcaattgagttatgtccagacatatttaaatagc

SEQ ID NO: 11 - DNA, 51 bp, Artificial Sequence
  Description of Artificial Sequence Synthetic oligonucleotide
atggataccggtggctatttaaatatgtgtggacataactcaattgcagcg

SEQ ID NO: 12 - protein, 17 aa, Artificial Sequence
  Description of Artificial Sequence Synthetic peptide
MDTGGYLNMCGHNSIAA

SEQ ID NO: 13 - DNA, 39 bp, Artificial Sequence; identical to SEQ ID NO: 9
  Description of Artificial Sequence Synthetic oligonucleotide

SEQ ID NO: 14 - DNA, 51 bp, Artificial Sequence
  Description of Artificial Sequence Synthetic oligonucleotide
atggataccggtggctatttaaatatgtctggacataactcaattgcagcg

SEQ ID NO: 15 - DNA, 51 bp, Artificial Sequence; identical to SEQ ID NO: 11
  Description of Artificial Sequence Synthetic oligonucleotide

SEQ ID NO: 16 - protein, 17 aa, Artificial Sequence
  Description of Artificial Sequence Synthetic peptide
MDTGGYLNMSGHNSIAA

SEQ ID NO: 17 - DNA, 51 bp, Artificial Sequence
  Description of Artificial Sequence Synthetic oligonucleotide
gtgatatttggcaatcgccaggcggatcgctctccatgtgggacaggcacc

SEQ ID NO: 18 - protein, 17 aa, Artificial Sequence
  Description of Artificial Sequence Synthetic peptide
VIFGNRQADRSPCGTGT

SEQ ID NO: 19 - DNA, 30 bp, Artificial Sequence; identical to SEQ ID NO: 7
  Description of Artificial Sequence Synthetic oligonucleotide

SEQ ID NO: 20 - DNA, 51 bp, Artificial Sequence; identical to SEQ ID NO: 17
  Description of Artificial Sequence Synthetic oligonucleotide

SEQ ID NO: 21 - DNA, 51 bp, Artificial Sequence
  Description of Artificial Sequence Synthetic oligonucleotide
gtgatatttggcaatcgccaggcggatcgctctccaagcgggacaggcacc

SEQ ID NO: 22 - protein, 17 aa, Artificial Sequence
  Description of Artificial Sequence Synthetic peptide
VIFGNRQADRSPSGTGT

SEQ ID NO: 23 - protein, 16 aa, Artificial Sequence
  Description of Artificial Sequence Synthetic peptide motif
XXXHXXGXXXXXXXXG

SEQ ID NO: 24 - protein, 14 aa, Artificial Sequence
  Description of Artificial Sequence Synthetic peptide motif
DRSPXGXXXXAXXA

SEQ ID NO: 25 - protein, 5 aa, Artificial Sequence
  Description of Artificial Sequence Synthetic peptide
EPRGH

SEQ ID NO: 26 - protein, 6 aa, Artificial Sequence
  Description of Artificial Sequence Synthetic peptide
EPRGSD

SEQ ID NO: 27 - protein, 6 aa, Artificial Sequence
  Description of Artificial Sequence Synthetic peptide
EPRGND

SEQ ID NO: 28 - DNA, 1598 bp, Trypanosoma cruzi
cctttttctttttaaaaacaaaaaaaattccggggggaatatggaacagggtatatgcgtaaaagtgtctgtcccaaacaaaaattttttttttccgccttcccattttttttttttttttgtgtgtttcccttgatctctcgaacagggcaggaaaagcttctgtttgaccaaaaatataaaattattaagggcgagaaaaaagaaaagaaaaaaaatcaacgagcaaacaggagagaacaccaacaaaaaagggaaattatgcgatttaagaaatcattcacatgcatcgacatgcatacggaaggtgaagcagcacggattgtgacgagtggtttgccacacattccaggttcgaatatggcggagaagaaagcatacctgcaggaaaacatggattatttgaggcgtggcataatgctggaaccacgtggtcatgatgatatgtttggagcctttttatttgaccctattgaagaaggcgctgacttgggcatggtattcatggataccggtggctatttaaatatgtgtggacataactcaattgcagcggttacggcggcagttgaaacgggaattgtgagcgtgccggcgaaggcaacaaatgttccggttgtcctggacacacctgcggggttggtgcgcggtacggcacaccttcagagtggtactgagagtgaggtgtcaaatgcgagtattatcaatgtaccctcatttttgtatcagcaggatgtggtggttgtgttgccaaagccctatggtgaagtacgggttgatattgcatttggaggcaattttttcgccattgttcccgcggagcagttgggaattgatatctccgttcaaaacctctccaggctgcaggaggcaggagaacttctgcgtactgaaatcaatcgcagtgtgaaggttcagcaccctcagctgccccatattaacactgtggactgtgttgagatatacggtccgccaacgaacccggaggcaaactacaagaacgttgtgatatttggcaatcgccaggcggatcgctctccatgtgggacaggcaccagcgccaagatggcaacactttatgccaaaggccagcttcgcatcggagagacttttgtgtacgagagcatactcggctcactcttccagggcagggtacttggggaggagcgaataccgggggtgaaggtgccggtgaccaaagatgccgaggaagggatgctcgttgtaacggcagaaattactggaaaggcttttatcatgggtttcaacaccatgctgtttgacccaacggatccgtttaagaacggattcacattaaagcagtagatctggtagagcacagaaactattggggaacacgtgcgaacaggtgctgctacgtgaagggtattgaatgaatcgtttttttttatttttattttttatttttattagtgcattattattaaattttttttttgttttggggtttcaacggtaccgcgttgggagcagggaagcgatagcggccggacaattttttgcttttattttcattttcatcttcctacccaacccccttggttccaccggtcgcggcggggtcttgtgggtggagg

SEQ ID NO: 29 - DNA, 1582 bp, Trypanosoma cruzi
gtgtgttcaacagttttgtttccttttttctctttttctctttccatcatacatacatacatacatacatatatatatctgcgtagatatgcacatgcgtatatgcgtgaagagtgtctgtcccaacattttttttttttttttgtgtgttttcccttgattcccgaacgggcaggaaaagcttctgtttgaccaaaaatataaaattattaagggcgagaaaagaaaaaaaaaatcaaccgaggagacaacaccaacaaaaaagggaaattatgcgatttaagaaatcattgacatgcatcgacatgcatacggaaggtgaagcagcacggattgtgacgagtggtttgccacacattccaggttcgaatatggcggagaagaaagcatacctgcaggaaaacatggattatttgaggcgtggcataatgctggagccacgtggtcatgatgatatgtttggagcctttttatttgaccctattgaagaaggcgctgacttgggcatcgtattcatggataccggtggctatttaaatatgtgtggacataactcaattgcagcggttacggcggcagtggaaacgggaattttgagcgtgccggcgaaggcaacaaatgttccggttgtcctggacacacctgcggggttggtgcgcggtacggcacaccttcagagtggtactgagagtgaggtgtcaaatgcgagtattatcaatgtgccctcatttttgtatcagcaggatgtggtgattgttttgccaaagccctatggtgaggtacgggttgatattgcatttggaggcaattttttcgccattgttcccgcggagcacttgggaattgatatctccgttcaaaacctctccaggctgcaggaggcaggagaacttctgcgtactgaaatcaatcgcagtgtgaaggttcagcaccctcagctgccccatattaacactgtggactgtgttgagatatacggtccgccaacgaacccggaggcaaaatacaagaacgttgtgatatttggcaatcgccaggcggatcgctctccatgtgggacaggcaccagcgccaagatggcaacactttatgccaaaggccagcttcgcatcggagagacttttgtgtacgagagcatactcggctcactcttccagggcagggtacttggggaggagcgaataccgggggtgaaggtgccggtgaccaaagatgccgaggaagggatgctcgttgtaacgacagaaattactggaaaggcttttatcatgggtttcaacaccatgctgtttgacccaacggatccgttcttaaacggattcacactaaagcggtagatctggtagagcacagaaactattggggaacacgtgcgaacaggtgctgctacgtaaagggtattgaatgaatcgtttttttttttttttttttattagtgcattatttttttttttttttgttttggggtttcaacggtaccacgttgggagcagggaaacgatagcggccggacaattttttacttttattttcattttcaccttcctacccaacccccttggttccaccggtcgcggcgggg

SEQ ID NO: 30 - protein, 423 aa, Trypanosoma cruzi [sequence omitted]

SEQ ID NO: 31 - protein, 354 aa, Trypanosoma cruzi
MRFKKSLTCIDMHTEGEAARIVTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVIVLPKPYGEVRVDIAFGGNFFAIVPAEHLGIDISVQNLSRLQEAGELLRTEINRSVKVQHPQLPHINTVDCVEIYGNATNPEAKYKNVVIFGNRQADRSPCGTGTSAKMATLYAKGQLRIGETFVYESILGSLFQGRVLGEERIPGVKVPVTKDAEEGMLVVTTEITGKAFIMGFNTMLFDPTDPFLNGFTLKR

SEQ ID NO: 32 - protein, 335 aa, Clostridium sticklandii
MKFSKGIHAIDSHTMGEPTRIVVGGIPQINGETMADKKKYLEDNLDYVRTALMHEPRGHNDMFGSIITSSNNKEADFGIIFMDGGGYLNMCGHGSIGAATVAVETGMVEMVEPVTNINMEAPAGLIKAKVMVENEKVKEVSITNVPSFLYMEDAKLEVPSLNKTITFDISFGGSFFAIIHAKELGVKVETSQVDVLKKLGIEIRDLINEKIKVQHPELEHIKTVDLVEIYDEPSNPEATYKNVVIFGQGQVDRSPCGTGTSAKLATLYKKGHLKIDEKFVYESITGTMFKGRVLEETKVGEFDAIIPEITGGAYITGFNHFVIDPEDPLKYGFTV

SEQ ID NO: 33 - protein, 354 aa, Homo sapiens
MESALAVPWLPPHDPGTPVLSVVDMHTGGEPLRIVLAGCPEVSGPTLLAKRRYMRQHLDHVRRRLMFEPRGHRDMYGAVLVPSELPDAHLGVLFLHNEGYSSMCGHAVLALGRFALDFGLVPAPPAGTREARVNIHCPCGLVTAFVACEDGRSHGPVRFHSVPAFVLATDLMVDVPGHGKVMVDIAYGGAFYAFVTAEKLGLDICSAKTRDLVDAASAVTEAVKAQFKINHPDSEDLAFLYGTILTDGKDAYTKEPTTNICVFADEQVDRSPTGSGVTARIALQYHKGLLELNQMRAFKSSATGSVFTGKAVREAKCGDFKAVIVEVSGQAHYTGTASFIIEDDDPLRDGFLLK

SEQ ID NO: 34 - protein, 354 aa, Homo sapiens
MESALAVPRLPPHDPGTPVLSVVDMHTGGEPLRIVLAGCPEVSGPTLLAKRRYMRQHLDHVRRRLMFEPRGHRDMYGAVLVPSELPDAHLGVLFLHNEGYSSMCGHAVLALGRFALDFGLVPAPPAGTREARVNIHCPCGLVTAFVACEDGRSHGPVRFHSVPAFVLATDLMVDVPGHGKVMVDIAYGGAFYAFVTAEKLGLDICSAKTRDLVDAASAVTEAVKAQFKINHPDSEDLAFLYGTILTDGKDAYTKEPTTNICVFADEQVDRSPTGSGVTARIALQYHKGLLELNQMRAFKSSATGSVFTGKAVREAKCGDFKAVIVEVSGQAHYTGTASFIIEDDDPLRDGFLLK

SEQ ID NO: 35 - protein, 354 aa, Mus musculus
MEAALAVTRLPPNDPRTPALSVVDMHTGGEPLRIVHAGCPEVAGPTLLAKRRYMRQHLDYIRRRLVFEPRGHRDMYGAILVPSELPDAHLGVLFLHNEGYSSMCGHAVLALGRFALDFGLVPAPPKGAREAQVNIHCPCGLVTAFVECEGGRSCGPVRFHSVPAFVLASDLTVDVPGHGKVLVDIAYGGAFYAFVSAEKLGLDVCSAKTRDLVDAASALTGAVKAQFKINHPESEDLGFLYGSILTDGKDAYSEEATTNICVFADEQVDRSPTGSGVTARIALQYHKGLLQLNQTRAFKSSATGSVFTGCAVREAKCGDFKAVIVEVAGQAHYTGTANLTVEDGDPLRDGFLLK

SEQ ID NO: 36 - protein, 354 aa, Mus musculus
MEAALAVTRLPPHDSRTPALSVVDMHTGGEPLRIVHAGCPEVAGPTLLAKRRYMRQHLDYIRRRLVFEPRGHRDMYGAILMPSELPDAHLGVLFLHNEGYSSMCGHAVLALGRFALDFGLVPAPPEGAREAQVNIHCPCGLVTAFVECEGGRSCGPVRFHSVPAFVLASDLTVDVPGHGKVLVDIAYGGAFYAFVSAEKLGLDVCSAKTRDLVDAASALTGAVKAQFKINHPESEDLGFLYGSILTDGKDAYSEEATTNICVFADEQVDRSPTGSGVTARIALQYHKGLLQLNQTRAFKSSATGSVFTGCAVREAKCGDFKAVIVEVAGQAHYTGTANLTVEDGDPLRDGFLLK

SEQ ID NO: 37 - protein, 333 aa, Rhizobium loti
MAKKSFFCIDGHTCGNPVRLVAGGGPLLEGSTMMERRAHFLAEYDWIRTGLMFEPRGHDVMSGSILYPPTREDCDIAILFIETSGCLPMCGHGTIGTVTMAIEHGLIKPKTPGVLRLDTPAGLVIAEYKQVGEYVEEVRITNVPSFLHAEGLTVECPGLGEITVDVAYGGNFYAIVEPQENYRDMADHSAGDLIAWSPVVRQRLNEKYSFVHPENPGINRLSHMLWTGKPTVEGADARNAVFYGDKAIDRSPCGTGTSARMAQLHAKGKLKAGDAFVHESIIGSLFKGKVEKEVTVAGKPAIIPSIGGWARLTGLNTIFIDDRDPFAHGFVVT

SEQ ID NO: 38 - protein, 333 aa, Brucella melitensis
MARHSFFCVDGHTCGNPVRLVAGGGPNLNGSTMMEKCAHFLAEYDWIRTGLMFEPRGHDMMSGSILYPPTRPDCDVAVLFIETSGCLPMCGHGTIGTVTMAIEQGLVTPKTPGKLNLDTPAGLVAIEYEQDGQYVERVRLTNVPAFLYAEGLEVECPDLGPIKVDVAYGGNFYAIVEPQENYTDMDDYSALQLIAWSPVLRQRLNEKYKFQHPELPDINRLSHILWTGKPKHPQAHARNAVFYGDKAIDRSPCGTGTSARMAQLAAKGKLKPGDEFIHESIIGSLFHGRVERAAEVAGRPAIVPSIAGWARMTGYNTIFIDDRDPFAHGFSAA

SEQ ID NO: 39 - protein, 333 aa, Rhizobium meliloti
MATHTFSCIDGHTCGNPVRLVSGGGPRLEGANMLEKRAHFLKEFDWIRTGLMFEPRGHDMMSGSILYPPTRPDCDVAVLFIETSGCLPMCGHGTIGTITMGIENGLITPREPGKLSIDAPAGKVDITYRQEGRFVEEVRLTNVPSFLYAEGLAAEVEGLGEIVVDVAYGGNFYAIVEPQKNFRDMADHTAGELVGWSPKLRAALNAKYEFVHPEHPEIRGLSHIQWTGKPTQPEAHARNAVFYGEKAIDRSPCGTGTSARIAQLAAKGKLKVGDEFVHESIIGSLFKGRVEAAAKVADRDAIIPSIAGWARMTGINTIFIDDRDPFAHGFVVR

SEQ ID NO: 40 - protein, 332 aa, Agrobacterium tumefaciens
MRHSFFCIDSHTCGNPVRLVAGGGPLLPHLPISERRDLFVRNHDWVRQALMFEPRGHDIMSGAVIYPAYRDDCDFAVIFIEVSGCLPMCGAGTIGLVTAAIEEGLVTPRIPGRLSIETPAGKVDIQYDKPGEFVESVRIFNVASYLHAADVEVNVPGLGKLVVDIAYGGNYYAVIEPQVGWPGLDGMTAGDVVDLSQKLRDALGTICDPVHPDDERIRGVHHAIWCDRPVSAEADGRGAVFYGDKAIDRSPGGTGTSARMAQLHGKGRLKAGETFRQESLIGTIFEGKVEEETTVGSFSGIRPSIGGWARIIGHNTIFVDDRDPLAHGFQVR

SEQ ID NO: 41 - protein, 312 aa, Xanthomonas campestris
MHTIDVIDSHTAGEPTRVVLAGFPDLGDGDLAQCRERFRSDFDHWRSAIACEPRGSDTMVGALLLPPRDPSACTGVIFFNNVGYLGMCGHGTIGVVRTLAELGRIAPGQHRIETPVGTVGVALADDGTVSIDNVESYRHAAGVEVDVPGHGRVRGDVAWGGNWFFITEQAPCALGLAQQRELTAYTEAIRLALEAAGITGEAGGEIDHIEISGVAPDGSGAARNFVLCPGLAYDRSPCGTGTSAKLACLAADGKLAEGERWLQQGILGSAFEGSYRHSGRGIAPRISGHAFITARSQLLIDPADPFAWGIVA

SEQ ID NO: 42 - protein, 312 aa, Xanthomonas axonopodis
MHTIDVIDSHTAGEPTRVVLSGFPDLGDGDLAQCRERFRSEFDHWRSAIACEPRGSDTMVGALLLPPRDPSACTGVIFFNNVGYLGMCGHGTIGVVRTLAELGRIAPGQHRIETPVGTVGVELADDGTVSVDNVESYRFASGVEVEVPGHGRVCGDVAWGGNWFFITEHAPCALDLAHQRELTAYTEAIRLALEAAGITGEAGGEIDHIEVNGAAPDGSGVARNFVLCPGLAYDRSPCGTGTSAKLACLAADGKLAEGERWVQQGILGSAFEGNYRLSGRGIAPRISGRAYITARAQLVIDPADPFAWGIVA

SEQ ID NO: 43 - protein, 314 aa, Pseudomonas aeruginosa
MQRIRIIDSHTGGEPTRLVIGGFPDLGQGDMAERRRLLGERHDAWRAACILEPRGSDVLVGALLCAPVDPEACAGVIFFNNSGYLGMCGHGTIGLVASLAHLGRIGPGVHRIETPVGEVEATLHEDGSVSVRNVPAYRYRRQVSVEVPGIGRVSGDIAWGGNWFFLVAGHGQRLAGDNLDALTAYTVAVQQALDDQDIRGEDGGAIDHIELFADDPHADSRNFVLCPGKAYDRSPCGTGTSAKLACLAADGKLLPGQPWRQASVIGSQFEGRYEWLDGQPGGPIVPTIRGRAHVSAEATLLLADDDPFAWGIRR

SEQ ID NO: 44 - protein, 344 aa, Pseudomonas aeruginosa
MRSQRIVHIVSCHAEGEVGDVIVGGVAAPPGATLWEQSRWIARDQDLRNFVLNEPRGGVFRHANLLVPAKDPRAQMGWIIMEPADTPPMSGSNSLCVATVLLDSGILPMREPLTRLLLEAPGGLIEARAECRDGKAERVEIRNVPSFADRLDAWIEVEGLGSLQVDTAYGGDSFVIADARRLGFALRADEAAELVATGLKITHAANEQLGFRHPTNPDWDHLSFCQLAAPPERRDGVLGANNAVVIRPGKIDRSPCGTGCSARMAVLQAKGQLRVGERFVGRSIIGSEFHCHIESLTELGGRPAILPCLSGRAWITGIHQYLLDPDDPWPQGYRLSDTWPGGHC

SEQ ID NO: 45 - protein, 342 aa, Brucella melitensis
MRSTKVIHIVGCHAEGEVGDVIVGGVAPPPGETVWEQSRFIANDETLRNFVLNKPRGGVFRHVNLLVPPKDPRAQMGFIIMEPADTPPMSGSNSICVSTVLLDSGIIAMQEPVTHMVLEAPGGIIEVEAECRNGKAERISVRNVPSFADRLDAPLDVTGLGTIMVDTAYGGDSFVIVDAAQIGMKIEPGQARELAEIGVKITKAANEQLGFRHPERDWRHISFCQITEPVTREGDVLTGVNTVAIRPAKFDRSPTGTGCSARMAVLHAKGQMKAGERFIGKSVLGTEFHCRLDKVLELGGKPAISPIISGRAWVTGTSQLMLDPSDPFPHGYRLSDTWPRDE

SEQ ID NO: 46 - protein, 342 aa, Agrobacterium tumefaciens
MRSIKTVHVISAHAEGEVGDVIVGGVKPPPGETIWEQSRFIARDETLRNFVLNEPRGGVFRHVNLLVPPKHPDADAAFIIMEPEDTPPMSGSNSICVSTVLLDGGIVPMQEPETHMLLEAPGGLVKVRAECRNGKAERIFVQNLPSFAAKLDAELEVEGLGKLKVDTAYGGDSFVIVDAEAMGFSLKPEEAHEIARLGVRITNAANKALGFDHPENPDWRHFSFCLFAGKVERTAEGLRAGAAVAIQPGKVDRSPTGTALSARMAVLHARGEMKEGETLTAVSLIGSTFTGRILGTTTVGDRPAILPEISGRGWITGIHQHMLDPSDPWPEGYRLTDTWGAR

SEQ ID NO: 47 - protein, 342 aa, Rhizobium meliloti
MRSTKTIHVISAHAEGEVGDVIVGGVAPPPGDTIWEQSRWIAREQTLRNFVLNEPRGGVFRHVNLLVPPKHPDADAAFIIMEPEDTPPMSGSNSICVSTVLLDSGILPMKEPVTEITLEAPGGLVRVRAECRDGKAERIFVENLPSFAERLDAKLEVEGLGTLTVDTAYGGDSFVIVDAAAMGFALKPDEAHDIARLGVRITNAANAKLGFHHPENPDWRHFSFCLFAGPVERTAEGLRAGAAVAIQPGKVDRSPTGTALSARMAVLHARGQMGLSDRLTAVSLIGSTFSGRILGTTEVGGRPAVLPEISGRAWITGTHQHMLDPSDPWPEGYRLTDTWGAR

SEQ ID NO: 48 - protein, 346 aa, Rhizobium loti
MRSKTSIRVVGCHAEGEVGDVIIGGVLPPAGRTMMDKMITMERDHDHIRRMLICEPRGSVARHVNLLVPSTREDCAAGAIIMEPTEYPPMSGSNTICVATVLLETGMVPMQEPETRFKLDMPGGVIEVRAQCRDGKCVSITLRNAPAFVDRLDASIEVEGLGTLTVDIAYGGMFYAIVDAKALGFSIAPDEARELAVAGEKIRRAAREQLDVVHPQFDHVRGVSIVQFAMPFQGPGNVTRNTCIVSPGRSDRSPTGTGTSARMAVLQARGLMGVGDVLIHESIIGSRFTGRIVELAEIAGRKAIVPEITGRAWITGEHSYYLDPTDPYPQGYVLSDTWGTSTSVKQ

SEQ ID NO: 49 - protein, 339 aa, Xanthomonas axonopodis
MRWSKQFSVVDCHAEGEVGKVIVGGVGNIPGTTMFEKKLYLEQHRDDIRKLVLQEPRGATWHNANIILPPSHPEASMGFVILEATEYPAMSGSNTICVATVLLETGILPMQEPITDLVLEAPAGLIRVRCDCKDGKVTRVKLVNQPAFVYHLDAKVEVAGIGTVSADIAFGGMTFALVDASSLGFEIVPAEARELCEYGQKIKAAAAEQLDVAFPGNPDMPGITMTQFTGPLSRADGKFFSRNTTIVSPGRCDRSPCGAGSSARLAALHAKGVLAKGDTLVHESIIGSRFECGIEDMSNVGDYPAVVPSIAGQAWISGLSQLGLDPSDPYAEGFTLADR

SEQ ID NO: 50 - protein, 333 aa, Streptomyces coelicolor
MRSTVCYHAVDSHTEGMPTRVITGGVGVLPGATMFERRQRFVAERDHLRTLLMCEPRGHASMSGAILQPPTRPDADYGVLFIEVSGCLPMCGHGTIGVATVLVETGMVEVTEPETTVRLDTPAGLVTARVRVRDGHAESVTLENVASYSHALDQVVDVPGHGEVRYDIAYGGNFYAFVRTDDLGIPFERAHKQPLLDAGLAVMDAINKQNPVSHPENPDIDVCHHVYLEAPGSTAEHSRHAMAIHPGWFDRSPCGTGTSARMAQLHARGLLPAGRDFVNESFIGSRFVGRVLGETTVGGRPAVLPSVTGRAWITGTAQYLLDPSDPYPAGFTL

SEQ ID NO: 51 - protein, 345 aa, Agrobacterium tumefaciens
MRWKRTLQLLDVHCEGEIGRVVTGGAPKIPGNTVAEQLHWMNTDPQGEALRRFLTLEPRGTPMGSVDLLLPPKHPDAHAAFVILQPDQAHASSGSNSICATTALLESGMVEMQEPETVIILETAAGLVKATATCRDGRCEKVKLTMVPSFVHELDVSIDTPEWGRVTMDISYGGIFYALVDVRQIGLTIEKANAAKLVAAGMTLKDLVNREMTVVHPEIPAISGVAYVMFRDVDADGSIRTCTTMWPGRADRSPCGTGNSANLATLYARGKVKVGDEYKSRSIIGSEFDVGLSAVTEVAGRPAVIPTIAGRGFTFGLHQVGLDPFDPLGDGFAMTDVWGPEAGNI

SEQ ID NO: 52 - protein, 354 aa, Trypanosoma cruzi
MRFKKSLTCIDMHTEGEAARIVTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVIVLPKPYGEVRVDIAFGGNFFAIVPAEHLGIDISVQNLSRLQEAGELLRTEINRSVKVQHPQLPHINTVDCVEIYGPPTNPEAKYKNVVIFGNRQADRSPCGTGTSAKMATLYAKGQLRIGETFVYESILGSLFQGRVLGEERIPGVKVPVTKDAEEGMLVVTTEITGKAFIMGFNTMLFDPTDPFLNGFTLKR

SEQ ID NO: 53 - DNA, 15 bp, Artificial Sequence
  Description of Artificial Sequence Synthetic oligonucleotide
tctccatgtgggaca

SEQ ID NO: 54 - protein, 5 aa, Artificial Sequence
  Description of Artificial Sequence Synthetic peptide
SPCGT

SEQ ID NO: 55 - DNA, 15 bp, Artificial Sequence
  Description of Artificial Sequence Synthetic oligonucleotide
tctccaagcgggaca

SEQ ID NO: 56 - protein, 5 aa, Artificial Sequence
  Description of Artificial Sequence Synthetic peptide
SPSGT

SEQ ID NO: 57 - protein, 146 aa, Trypanosoma cruzi
GEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPGVKVPVTK

SEQ ID NO: 58 - protein, 146 aa, Bacillus anthracis
GTVEADIAYGGNFYAIIDAKSVGLELVPEHASTIIDKAIHIRNIINERFEIIHPEYSFIRGLTHVEFYTDPTHESAHVKNTVVVPPGGIDRSPCGTGTSAKLAVLYANQKIEMNEEFVHESIVGSLFKGCVINTTNVANMEAVVTK

SEQ ID NO: 59 - protein, 117 aa, Trypanosoma cruzi
MRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNV

SEQ ID NO: 60 - protein, 117 aa, Bacillus anthracis
MRTQKVFTTIDTHTGGNPTRTLISGLPKLLGETMAEKMLHMKKEYDWIRKLLMNEPRGHDVMSGALLTDPCHPDADIGVIYIETGGYLPMCGHDTIGVCTALIESGLIPVVEPITSL

SEQ ID NO: 61 - protein, 145 aa, Trypanosoma cruzi
EVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPGVKVPVTK

SEQ ID NO: 62 - protein, 145 aa, Bacillus anthracis
EFQVDIAFGGAFYAVVDSKEFGLKVDFKDLSAIQQWGGKIKHYIESKMEVKHPLEEGLKGIYGVIFSDDPKGEGATLRNVTIFADGQVDRSPCGTGTSARIATLFEKGILQKGEIFIHECITDGEFEGEVLSVTAVHTYEAVVPK

SEQ ID NO: 63 - protein, 113 aa, Trypanosoma cruzi
MRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAK

SEQ ID NO: 64 - protein, 113 aa, Bacillus anthracis
MKVSKVYTTIDAHVAGEPLRIITGGVPEIKGETQLERRWYCMEHLDYLREVLMYEPRGHHGMYGCIITPPASAHADFGVLFMHNEGWSTMCGHGIIAVITVGIETGMFETKQK

SEQ ID NO: 65 - protein, 138 aa, Trypanosoma cruzi
YGEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERI

SEQ ID NO: 66 - protein, 138 aa, Clostridium botulinum
YGKLTLDISFGGSFFAMVDAEKVGIDISPANSQKLNDLGMKIVHAVNEQVEIKHPVLEHIKTVDLCEFYGPAKSEDADVQNVVVFGQGQVDRSPCGTGTSAKMALLYAQGKMKVGEEIVNESIICTKFKGKILEETKV

SEQ ID NO: 67 - protein, 118 aa, Trypanosoma cruzi
IMRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNV

SEQ ID NO: 68 - protein, 118 aa, Clostridium botulinum
IMRAIKTIQTIESHTMGEPTRIVIGGLPKVPGKTMAEKMEYLEENNDSLRTMLMSEPRGHNDMFGAIYTEPADETADLGIIFMDGGGYLNMCGHGSIGAATCAVEMGIVKVEEPYTNI

SEQ ID NO: 69 - protein, 224 aa, Trypanosoma cruzi
ADLGIVFMDTGGYLNMCGHNSIAAVTAAVETGILSVPAKATNVPVVLDTPAGLVRGTAHLQSGTESEVSNASIINVPSFLYQQDVVIVLPKPYGEVRVDIAFGGNFFAIVPAEHLGIDISVQNLSRLQEAGELLRTEINRSVKVQHPQLPHINTVDCVEIYGNATNPEAKYKNVVIFGNRQADRSPCGTGTSAKMATLYAKGQLRIGETFVYESILGSLFQGRV

SEQ ID NO: 70 - protein, 218 aa, Clostridium botulinum
ADLGIIFMDGGGYLNMCGHGSIGAATCAVEMGIVKVEEPYTNIKLEAPAGMINARVKVEDGKAKETSIVNVPAFLYKKDVEIDVPDYGKLTLDISFGGSFFAMVDAEKVGIDISPANSQKLNDLGMKIVHAVNEQVEIKHPVLEHIKTVDLCEFYGPAKSEDADVQNVVVFGQGQVDRSPCGTGTSAKMALLYAQGKMKVGEEIVNESIICTKFKGKI

SEQ ID NO: 71 - protein, 72 aa, Trypanosoma cruzi
PTNPEANYKNVVIFGNRQADRSPCGTGTSAKMATLYAKGQLRIGETFVYESILGSLFQGRVLGEERIPGVKV

SEQ ID NO: 72 - protein, 72 aa, Aspergillus fumigatus
PDDVQGAETGLCYFAENQIDRSPTGSCVIARMALAYAKGLRSLGQRWAYNSLVSNRFGTGAFSAEIVEEVTI

SEQ ID NO: 73 - protein, 34 aa, Trypanosoma cruzi
MAEKKAYLQENMDYLRRGIMLEPRGHDDMFGAFL

SEQ ID NO: 74 - protein, 34 aa, Aspergillus fumigatus
LLEQRDQAKQHHDHIRKCLMLEPRGHNGMYGAII

SEQ ID NO: 75 - protein, 29 aa, Trypanosoma cruzi
CIDMHTEGEAARIVTSGLPHIPGSNMAEK

SEQ ID NO: 76 - protein, 29 aa, Aspergillus fumigatus
CIDMHTTGEPTRIIYSGFPPLSGTLLEQR

SEQ ID NO: 77 - protein, 27 aa, Trypanosoma cruzi
VDIAFGGNFFAIVPAEQLGIDISVQNL

SEQ ID NO: 78 - protein, 27 aa, Aspergillus fumigatus
LDISYGGAFYAIVQASELGFSGGLRDL

SEQ ID NO: 79 - protein, 20 aa, Trypanosoma cruzi
SSIGSNKKAPNISSPRGSSI

SEQ ID NO: 80 - protein, 20 aa, Aspergillus fumigatus
SSVSGRMMAPYIPLPRGSSI

SEQ ID NO: 81 - protein, 146 aa, Trypanosoma cruzi; identical to SEQ ID NO: 57

SEQ ID NO: 82 - protein, 146 aa, Clostridium difficile
GTVKFDISFGGSFFAIIHASQLGLKIEPQNAGKLTELAMKLRDIINEKIEIQHPTLAHIKTVDLVEIYDEPTHPEATYKNVVIFGQGQVDRSPCGTGTSAKLATLHAKGELKVGEKFVYESILGTLFKGEIVEETKVADFNAVVPK

SEQ ID NO: 83 - protein, 117 aa, Trypanosoma cruzi; identical to SEQ ID NO: 59

SEQ ID NO: 84 - protein, 117 aa, Clostridium difficile
MKFSRSIQAIDSHTAGEATRIVVGGIPNIKGNSMPEKKEYLEENLDYLRTAIMLEPRGHNDMFGSVMTQPCCPDADFGIIFMDGGGYLNMCGHGTIGAMTAAIETGVVPAVEPVTHV

SEQ ID NO: 85 - protein, 139 aa, Trypanosoma cruzi
GEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPG

SEQ ID NO: 86 - protein, 139 aa, Brucella suis
GPIKVDVAYGGNFYAIVEPQENYTDMDDYSALQLIAWSPVLRQRLNEKYKFQHPELPDINRLSHILWTGKPKHPQAHARNAVFYGDKAIDRSPCGTGTSARMAQLAAKGKLKPGDEFIHESIIGSLFHGRVERAAEVAG

SEQ ID NO: 87 - protein, 106 aa, Trypanosoma cruzi
KKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVS

SEQ ID NO: 88 - protein, 106 aa, Brucella suis
RHSFFCVDGHTCGNPVRLVAGGGPNLNGSTMMEKRAHFLAEYDWIRTGLMFEPRGHDMMSGSILYPPTRPDCDVAVLFIETSGCLPMCGHGTIGTVTMAIEQGLVT

SEQ ID NO: 89 - protein, 109 aa, Trypanosoma cruzi
MRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVS

SEQ ID NO: 90 - protein, 109 aa, Rhodobacter sphaeroides
MRVQDVYNVIYTHTEGEPLCIIYSGVPYPAGSTILEKRAFLEENYDWLRKALMREPRGHADMFGVFLTPPSSRDYDAGLIYIDGKEYSHMCGHGTIAVAMAMVANGLVA

SEQ ID NO: 91 - protein, 47 aa, Trypanosoma cruzi
KVQHPQLPHINTVDCVEIYGPPTNPEANYKNVVIFGNRQADRSPCGT

SEQ ID NO: 92 - protein, 47 aa, Rhodobacter sphaeroides
KSSTPTEAHINNLNFVTLWHKPPSRGWLYKNVHCFLEGQLDRLPGGT

SEQ ID NO: 93 - protein, 118 aa, Trypanosoma cruzi
RFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNVPV

SEQ ID NO: 94 - protein, 118 aa, Burkholderia pseudomallei
RDMKHIHIIDSHTGGEPTRVVVSGFPALGGGTMAERLAVLAREHDRYRAACILEPRGSDVLVGALLCEPVSAGAAAGVIFFNNAGYLGMCGHGTIGLVRTLHHMGRIGPGVHRIETPV

SEQ ID NO: 95 - protein, 63 aa, Trypanosoma cruzi
NPEANYKNVVIFGNRQADRSPCGTGTSAKMATLYAKGQLRIGETFVYESILGSLFQGRVLGEE

SEQ ID NO: 96 - protein, 63 aa, Burkholderia pseudomallei
DPEYDSRSFVLCPGHAYDRSPCGTGTSAKLACLAADGKLAAGVTWRQASVIGSVFSASYAAAE

SEQ ID NO: 97 - protein, 118 aa, Trypanosoma cruzi; identical to SEQ ID NO: 93

SEQ ID NO: 98 - protein, 118 aa, Burkholderia mallei; identical to SEQ ID NO: 94

SEQ ID NO: 99 - protein, 63 aa, Trypanosoma cruzi; identical to SEQ ID NO: 95

SEQ ID NO: 100 - protein, 63 aa, Burkholderia mallei
DPEYDSRSFVLCPGHAYDRSPCGTGTSAKLACLAADGKLVAGVTWRQASVIGSVFSASYAAAE

SEQ ID NO: 101 - protein, 115 aa, Trypanosoma cruzi
KSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNVPV

SEQ ID NO: 102 - protein, 115 aa, Pseudomonas putida
KQIHVIDSHTGGEPTRLVMKGFPQLRGRSMAEQRDELRELHDRWRRACLLEPRGNDVLVGALYCPPVSADATCGVIFFNNAGYLNMCGHGTIGLVASLQHMGLITPGVHKIDTPV

SEQ ID NO: 103 - protein, 58 aa, Trypanosoma cruzi
NPEANYKNVVIFGNRQADRSPCGTGTSAKMATLYAKGQLRIGETFVYESILGSLFQGR

SEQ ID NO: 104 - protein, 58 aa, Pseudomonas putida
DPNADSRNFVMCPGKAYDRSPCGTGTSAKLACLAADGKLAEGQTWVQASITGSQFHGR

SEQ ID NO: 105 - protein, 180 aa, Trypanosoma cruzi
SGLPHIPGSNMAEKKAYLQENMDYLRRGIMLEPRGHDDMFGAFLFDPIEEGADLGIVFMDTGGYLNMCGHNSIAAVTAAVETGIVSVPAKATNVPVVLDTPAGLVRGTAHLQSGTESEVSNASIINVPSFLYQQDVVVVLPKPYGEVRVDIAFGGNFFAIVPAEQLGIDISVQNLSRLQE

SEQ ID NO: 106 - protein, 164 aa, Leishmania major
TGFPELAGETIADKLDNLRTQHDQWRRACLLEPRGNDVLVGALYCAPVSADATCGVIFFNNAGYLGMCGHGTIGLVASLHHLGRIAPGVHKIDTPVGPVSATLHADGAVTLRNVPAYRYRQQVPVDVPGHGRVYGDIAWGGNWFFLVSDHGQALQMDNVEALTD

SEQ ID NO: 107 - protein, 68 aa, Trypanosoma cruzi
PTNPEANYKNVVIFGNRQADRSPCGTGTSAKMATLYAKGQLRIGETFVYESILGSLFQGRVLGEERIP

SEQ ID NO: 108 - protein, 68 aa, Leishmania major
PTTPTPTATSSCAQGKAYDRSPCGTGTNAKLACLAGDSKLAAGEPWLQVTITCRQFKRSYQWECKRVP

SEQ ID NO: 109 - protein, 28 aa, Trypanosoma cruzi
VTAEITGKAFIMGFNTMLFDPTDPFKNG

SEQ ID NO: 110 - protein, 27 aa, Leishmania major
VPPSITRRAYMTADSTLLIDQDPFAWG

SEQ ID NO: 111 - protein, 182 aa, Trypanosoma cruzi
VTSGLPHIPGSNMAEKKAYLQENMDYLRRGIMLEPRGHDDMFGAFLFDPIEEGADLGIVFMDTGGYLNMCGHNSIAAVTAAVETGIVSVPAKATNVPVVLDTPAGLVRGTAHLQSGTESEVSNASIINVPSFLYQQDVVVVLPKPYGEVRVDIAFGGNFFAIVPAEQLGIDISVQNLSRLQE

SEQ ID NO: 112 - protein, 166 aa, Leishmania major
VMTGFPELAGETIADKLDNLRTQHDQWRRACLLEPRGNDVLVGALYCAPVSADATCGVIFFNNAGYLGMCGHGTIGLVASLHHLGRIAPGVHKIDTPVGPVSATLHADGAVTLRNVPAYRYRQQVPVDVPGHGRVYGDIAWGGNWFFLVSDHGQALQMDNVEALTD

SEQ ID NO: 113 - protein, 142 aa, Trypanosoma cruzi
RIVTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVI

SEQ ID NO: 114 - protein, 137 aa, Trypanosoma brucei
RIITGGVPEIKGETQLERRAYCMEHLDYLREILMYEPRGHHGMYGCIITPPASAHADFGVLFMHNEGWSTMCGHGIIAVITVGIETGMFEVKGEKQNFIIDSPAGEVIAYAKYNGSEVESVSFENVPSFVYKKDVPI

SEQ ID NO: 115 - protein, 140 aa, Trypanosoma cruzi
VTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVI

SEQ ID NO: 116 - protein, 135 aa, Trypanosoma brucei
ITGGVPEIKGETQLERRAYCMEHLDYLREILMYEPRGHHGMYGCIITPPASAHADFGVLFMHNEGWSTMCGHGIIAVITVGIETGMFEVKGEKQNFIIDSPAGEVIAYAKYNGSEVESVSFENVPSFVYKKDVPI

SEQ ID NO: 117 - protein, 103 aa, Trypanosoma cruzi
FGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQL

SEQ ID NO: 118 - protein, 95 aa, Trypanosoma congolense
WGGNWFFLVSDHGHELQMDNVEALTDYTWAMLNALEAQGIRGADGALIDHIELFADDAHADSRNFVMCPGKAYDRSPCGTGTSAKLACLAADAKL

SEQ ID NO: 119 - protein, 55 aa, Trypanosoma cruzi
NVPSFLYQQDVVVVLPKPYGEVRVDIAFGGNFFAIVPAEQLGIDISVQNLSRLQE

SEQ ID NO: 120 - protein, 52 aa, Trypanosoma congolense
HVPAYRYRKQVPVEVPGHGVVLGDIAWGGNWFFLVSDHGHELQMDNVEALTD

SEQ ID NO: 121 - protein, 117 aa, Trypanosoma cruzi
LPKPYGEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLR

SEQ ID NO: 122 - protein, 116 aa, Trypanosoma vivax
LPHPYGKYAVISFGGSFFALIDAAQLQLTVDKGHLSTLQHVGGLLRDTLNRNVSVQHPQLPHINRIDCVEIYDPPTNPAASCKNVVIFGNSQVDRSPCGTGTCAKMALLYAKGKLK

SEQ ID NO: 123 - protein, 106 aa, Trypanosoma cruzi
REIMRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAV

SEQ ID NO: 124 - protein, 106 aa, Trypanosoma vivax
RVVMQFTGTMTCIDMHTAGEPARIVTSGFPNIPGASLVEKRDHLQRHMDHIRRRVMLEPRGHDNMFGAFLFYPLTDGADFSVIFMDAGGYLNMCGHNSIAIATAAV

SEQ ID NO: 125 - protein, 357 aa, Trypanosoma cruzi
KREIMRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNVPVVLDTPAGLVRGTAHLQSGTESEVSNASIINVPSFLYQQDVVVVLPKPYGEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPGVKVPVTKDAEEGMLVVTAEITGKAFIMGFNTMLFDPTDPFKNGFTLK

SEQ ID NO: 126 - protein, 337 aa, Vibrio parahaemolyticus
KERKMRQGTFFCIDAHTCGNPVRLVAGGVPPLEGNTMSEKRQYFLEHYDWIRQALMFEPRGHSMMSGSVVLPPCSDNADASILFIETSGCLPMCGHGTIGTVTTAIENRLITPKEEGRLILDVPAGQIEVHYQTKGDKVTSVKIFNVPAYLAHQDVTVEIEGLGEITVDVAYGGNYYVIVDPQENYAGLEHYSPDEILMLSPKVRTAVSKAVECIHPNDPTVCGVSHVLWTGKPTQEGATARNAVFYGDKALDRSPCGTGTSARMAQWHAKGKLKSGEDFVHESIIGSLFNGRIEGITEVNGQTAILPSIEGWAQVYGHNTIWVDDEDPYAYGFEVK

SEQ ID NO: 127 - protein, 14 aa, Artificial Sequence
  Description of Artificial Sequence Synthetic peptide motif
DRSPCGTGTSAKMA

SEQ ID NO: 128 - protein, 5 aa, Artificial Sequence
  Description of Artificial Sequence Synthetic peptide motif
NMCGH

SEQ ID NO: 129 - protein, 6 aa, Artificial Sequence
  Description of Artificial Sequence Synthetic 6xHis tag
HHHHHH

(19) United States
(12) Patent Application Publication
(10) Pub. No.: US 2012/0076815 A1
(43) Pub. Date: Mar. 29, 2012

(54) IDENTIFICATION AND CHARACTERIZATION OF RACEMASES, DEFINITION OF PROTEIN SIGNATURES, AND A TEST FOR DETECTING D-AMINO ACID AND FOR SCREENING MOLECULES CAPABLE OF INHIBITING THE ACTIVITY OF RACEMASE, ESPECIALLY PROLINE RACEMASE

(75) Inventors: Paola Minoprio, Villiers sur Marne (FR); Nathalie Chamond, Paris (FR); Wim M. Degrave, Rio de Janeiro (BR); Armand Berneman, Paris (FR)

(73) Assignee: Institut Pasteur

(21) Appl. No.: 12/895,268

(22) Filed: Sep. 30, 2010

Related U.S. Application Data
(63) Continuation of application No. 11/819,161, filed on Jun. 25, 2007, now abandoned.
Application No. 11/819,161 is a division of application No. 11/008,570, filed on Dec. 10, 2004, now Pat. No. 7,262,015.
Application No. 11/008,570 is a continuation of application No. 10/775,339, filed on Feb. 11, 2004, now abandoned.
(60) Provisional application No. 60/446,263, filed on Feb. 11, 2003.

(51) Int. Cl. A61K 39/005; C12N 15/63; C07K 4/12; C07K 14/44; C07K 16/20; C12N 1/21; C12Q 1/68; G01N 33/573; C12Q 1/533; C12Q 1/26; C12Q 1/30; C12Q 1/28; C12N 5/10; C12N 1/19; A61P 33/02; A61P 37/04; C12N 15/30

(52) U.S. Cl. 424/191.1; 536/23.7; 435/320.1; 530/300; 530/387.9; 435/252.33; 435/6.11; 435/7.1; 435/4; 435/25; 435/27; 435/28; 435/325; 435/254.2

(57) ABSTRACT

This invention provides identification and characterization of racemases and definition of protein signatures of these racemases. This invention also provides identification of nucleic acid molecules encoding a peptide consisting of a motif characteristic of the protein signatures, and to the peptides consisting of these motifs. Antibodies specific for the peptides and to immune complexes of these antibodies with the peptides are also provided. Further, the invention relates to methods and kits for detecting racemases using the nucleic acid molecules of the invention, as well as the peptides consisting of the motifs and antibodies to these peptides.

### CROSS-REFERENCE TO RELATED APPLICATION

This application is based on and claims the benefit of U.S. Provisional Application Ser. No. 60/446,263, filed Feb. 11, 2003 (Attorney Docket No. 03495.6087). The entire disclosure of this Provisional application is relied upon and incorporated by reference herein

### BACKGROUND OF THE INVENTION

This invention relates to the identification and characterization of racemases and definition of protein signatures of these racemases. More particularly, this invention relates to the identification of nucleic acid molecules encoding a peptide consisting of a motif characteristic of the protein signatures, and to the peptides consisting of these motifs. This invention also relates to antibodies specific for the peptides and to immune complexes of these antibodies with the peptides. Further, the invention relates to methods and kits for detecting racemases using the nucleic acid molecules of the invention, as well as the peptides consisting of the motifs and antibodies to these peptides.

D-amino acids have long been described in the cell wall of gram-positive and especially gram-negative bacteria, where they constitute essential elements of the peptidoglycan and as substitutes of cell wall techoic acids (1). Moreover, various types of D-amino acids were discovered in a number of small peptides made by a variety of microorganisms through non-ribosomal protein synthesis (2), that function mainly as antibiotic agents. However, these examples were considered exceptions to the rule of homochirality and a dogma persisted that only L-amino acid enantiomers were present in eukaryotes, apart from a very low level of D-amino acids from spontaneous racemization due to aging (3).

Recently, an increasing number of studies have reported the presence of various D-amino acids (D-aa) either as protein bound (4) or under free forms (5) in a wide variety of organisms, including mammals. The origin of free D-aa, is less clear than that of protein bound D-aa. For instance, in mammals, free D-aa may originate from exogenous sources (as described in (6), but the recent discovery of amino acid racemases in eukaryotes has also uncovered an endogenous production of D-aa, questioning their specific functions. Thus, the level of D-aspartate is developmentally regulated in rat embryos (7); the binding of D-serine to NMDA mouse brain receptors promotes neuromodulation (8),(9), and D-aspartate appears to be involved in hormonal regulation in endocrine tissues (10).

All amino acid racemases require pyridoxal phosphate as a cofactor, except proline and hydroxyproline racemases, which are cofactor-independent enzymes. For example, two reports have been published addressing the biochemical and enzymatic characteristics of the proline racemase from the gram-positive bacterium Clostridium sticklandii (11,12). A reaction mechanism was proposed whereby the active site Cys²⁵⁶ forms a half-reaction site with the corresponding cysteine of the other monomer in the active, homodimeric enzyme.

Although a variety of racemases and epimerases has been demonstrated in bacteria and fungi, the first eukaryotic amino acid (proline) racemase isolated from the infective metacyclic forms of the parasitic protozoan Trypanosoma cruzi, the causative agent of Chagas' disease in humans (13), was recently described. This parasite-secreted proline racemase (TcPRAC) was shown to be a potent mitogen for host B cells and to play an important role in T. cruzi immune evasion and persistence through polyclonal lymphocyte activation (13). This protein, previously annotated as TcPA45, with monomer size of 45 kDa, is only expressed and released by infective metacyclic forms of the parasite (13).

The genomic organization and transcription of TcPRAC proline racemase gene indicated the presence of two homologous genes per haploid genome (TcPRACA and TcPRACB). Furthermore, localization studies using specific antibodies directed to 45 kDa-TcPRAC protein revealed that an intracellular and/or membrane associated isoform, with monomer size of 39 kDa is expressed in non-infective epimastigote forms of the parasite.

Computer-assisted analysis of the TcPRACA gene sequence suggested that it could give rise to both isoforms (45 kDa and 39 kDa) of parasite proline racemases through a mechanism of alternative trans-splicing, one of which would contain a signal peptide (13). In addition, preliminary analysis of putative TcPRACB gene sequences had revealed several differences that include point mutations as compared to TcPRACA, but that also suggest that TcPRACB gene could only encode an intracellular isoform of the enzyme as the gene lacks the export signal sequence. Any of these molecular mechanisms per se would ensure the differential expression of intracellular and extracellular isoforms of proline racemases produced in different T. cruzi developmental stages.

The process of production of a D-amino acid by using a L-amino acid source comprises the use of an amino acid racemase specific for the amino acid of interest, the racemase being produced from a recombinant expression system containing a vector having a polynucleotide sequence encoding the enzyme. In prokaryotic hosts, the racemases are known to be implicated in the synthesis of D-amino acids and/or in the metabolism of L-amino acids. For instance, the presence of free D-amino acids in tumors and in progressive autoimmune and degenerative diseases suggests the biological importance of eukaryotic amino acid racemases. It is well known that proteins or peptides containing D-amino acids are resistant to proteolysis by host enzymes. In addition, such proteins containing D-amino acids, at least one D-amino acid residue, can display antibiotic or immunogenic properties.

There is a growing interest in the biological role of D-amino acids, either as free molecules or within polypeptide chains in human brain, tumors, anti-microbial and neuropeptides, suggesting widespread biological implications. Research on D-amino acids in living organisms has been hampered by their difficult detection. There exists a need in the art for the identification of racemases and the identification of their enzymatic properties and their specificity for other compounds.

Although much progress has been made concerning prophylaxis of Chagas' disease, particularly vector eradication, additional cases of infection and disease development still occur every day throughout the world. Whilst infection was largely limited in the past to vector transmission in endemic areas of Latin America, its impact has increased in terms of congenital and blood transmission, transplants and recrudescence following immunosuppressive states. Prevalence of Chagas' disease in Latin America may reach 25% of the population, as is the case of Bolivia, or yet 1%, as observed in Mexico. From the 18-20 million people already infected with the parasite Trypanosoma cruzi, more than 60% live in Brazil and WHO estimates that 90 million individuals are at risk in South and Central America.

Some figures obtained from a recent census in USA, for instance, revealed that the net immigration from Mexico is about 1000 people/day, of those 5-10 individuals are infected by Chagas' disease. The disease can lie dormant for 10-30 years and as an example of many other progressive chronic pathologies it is characterized by being “asymptomatic”. Although at the 1990's, blood banks increased their appeals to Hispanics (50% of Bolivian blood is contaminated), panels of Food and Drug Administration (FDA) have recommended that all donated blood be screened for Chagas. Today, FDA has not yet approved an ‘accurate’ blood test to screen donor blood samples. This allegation seriously contrasts with the more than 30 available tests used in endemic countries. Additionally, recent reports on new insect vectors adapted to the parasite and domestic animals infected in more developed countries like USA, and the distributional predictions based on Genetic Algorithm for Rule-set Prediction models indicate a potentially broad distribution for these species and suggest additional areas of risk beyond those previously reported emphasizing the continuing worldwide public health issue.

To date, two drugs are particularly used to treat Trypanosoma cruzi infections. Nifurtimox (3-methyl-4-5′-nitrofurfurylidene-amino tetrahydro 4H-1,4-thiazine-1,1-dioxide), a nitrofurane from Bayer, known as Lampit, was the first drug to be used since 1967. After 1973, Benznidazol, a nitroimidazol derivative, known as Rochagan or Radanyl (N-benzyl-2-nitro-1-imidazol acetamide) was produced by Hoffman-La-Roche and is consensually the drug of choice. Both drugs are trypanosomicides and act against intracellular or extracellular forms of the parasite. Adverse side-effects include a localized or generalized allergic dermopathy, peripheral sensitive polyneuropathy, leucopenia, anorexia, digestive manifestations and rare cases of convulsions which are reversible by interruption of treatment. The most serious complications include agranulocytosis and trombocytopenic purpura.

Unquestionably, the treatment is efficient and should be applied in acute phases of infection, in children, and in cases where reactivation of parasitaemia is observed following therapy with immunosuppressive drugs or organ transplantation procedures. Some experts recommend that patients in indeterminate and chronic phases should also be treated. However, close to a hundred years after the discovery of the infection and its consequent disease, researchers still maintain divergent points of view concerning therapy against the chronic phases of the disease. As one of the criteria of cure is based on the absence of the parasite in the blood, it is very difficult to evaluate the efficacy of the treatment in indeterminate or chronic phases. Because the indeterminate form is asymptomatic, it is impossible to clinically evaluate the cure. Furthermore, a combination of serology and more sensitive advanced molecular techniques will be required and still may not be conclusive. The follow-up of patients for many years is then inevitable to objectively ascertain the cure.

Chagas' disease was recently considered as a neglected disease and DND-initiative (Drug for Neglected Diseases Initiative, DNDi) wishes to support drug discovery projects focused on the development of effective, safe and affordable new drugs against trypanosomiasis. Since current therapies remain a matter of debate, may be inadequate in some circumstances, are rather toxic and may be of limited effectiveness, the characterization of new formulations and the discovery of parasite molecules capable of eliciting protective immunity are absolutely required and must be considered as priorities.

### SUMMARY OF THE INVENTION

This invention aids in fulfilling these needs in the art. It has been discovered that the TcPRAC genes in T. cruzi encode functional intracellular or secreted versions of the enzyme exhibiting distinct kinetic properties that may be relevant for their relative catalytic efficiency. While the K_{M} of the enzyme isoforms were of a similar order of magnitude (29-75 mM), Vₘₐₓ varied between 2×10⁻⁴ to 5.3×10⁻⁵ mol of L-proline/sec/0.125 μM of homodimeric recombinant protein. Studies with the enzyme specific inhibitor and abrogation of enzymatic activity by site-directed mutagenesis of the active site Cys³³⁰ residue, reinforced the potential of proline racemase as a critical target for drug development against Chagas' disease.

This invention provides a purified nucleic acid molecule encoding a peptide consisting of a motif selected from SEQ ID NOS: 1, 2, 3, or 4.

This invention also provides a purified nucleic acid molecule that hybridizes to either strand of a denatured, double-stranded DNA comprising this nucleic acid molecule under conditions of moderate stringency.

In addition, this invention provides a recombinant vector that directs the expression of a nucleic acid molecule selected from these purified nucleic acid molecules.

Further, this invention provides a purified polypeptide encoded by a nucleic acid molecule selected from the group consisting of a purified nucleic acid molecule coding for
(a) a purified polypeptide consisting of Motif I (SEQ ID NO:1);
(b) a purified polypeptide consisting of Motif II (SEQ ID NO:2);
(c) a purified polypeptide consisting of Motif III (SEQ ID NO:3); and
(d) a purified polypeptide consisting of Motif III* (SEQ ID NO:4).

Purified antibodies that bind to these polypeptides are provided. The purified antibodies can be monoclonal antibodies. An immunological complex comprises a polypeptide and an antibody that specifically recognizes the polypeptide of the invention.

A host cell transfected or transduced with the recombinant vector of the invention is provided.

A method for the production of a polypeptide consisting of SEQ ID NOS: 1, 2, 3, or 4, comprises culturing a host cell of the invention under conditions promoting expression, and recovering the polypeptide from the host cell or the culture medium. The host cell can be a bacterial cell, parasite cell, or eukaryotic cell.

A method of the invention for detecting a racemase encoded by a nucleotide sequence containing a subsequence encoding a peptide selected from SEQ ID NO: 1, 2, 3, or 4, comprises:
(a) contacting the nucleotide sequence with a primer or a probe, which hybridizes with the nucleic acid molecule of the invention;
(b) amplifying the nucleotide sequence using the primer or the probe; and
(c) detecting a hybridized complex formed between the primer or probe and the nucleotide sequence.

This invention provides a method of detecting a racemase encoded by a nucleotide sequence containing a subsequence encoding a peptide selected from SEQ ID NO: 1, 2, 3, or 4. The method comprises:
(a) contacting the racemase with antibodies of the invention; and
(b) detecting the resulting immunocomplex.

A kit for detecting a racemase encoded by a nucleotide sequence containing a subsequence encoding a peptide selected from SEQ ID NO: 1, 2, 3, or 4, comprises:
(a) a polynucleotide probe or primer, which hybridizes with the polynucleotide of the invention; and
(b) reagents to perform a nucleic acid hybridization reaction.

This invention also provides a kit for detecting a racemase encoded by a nucleotide sequence containing a subsequence encoding a peptide selected from SEQ ID NO: 1, 2, 3, or 4. The kit comprises:
(a) purified antibodies of the invention;
(b) standard reagents in a purified form; and
(c) detection reagents.

An in vitro method of screening for an active molecule capable of inhibiting a racemase encoded by a nucleotide sequence containing a subsequence encoding a peptide selected from SEQ ID NO: 1, 2, 3, or 4, comprises:
(a) contacting the active molecule with the racemase;
(b) testing the capacity of the active molecule, at various concentrations, to inhibit the activity of the racemase; and
(c) choosing the active molecule that provides an inhibitory effect of at least 80% on the activity of any proline racemase.

In a preferred embodiment of the invention the racemase is a proline racemase.

An immunizing composition of the invention contains at least a purified polypeptide of the invention, capable of inducing an immune response in vivo, and a pharmaceutical carrier.

### BRIEF DESCRIPTION OF THE DRAWINGS

This invention will be understood with reference to the drawings in which:

FIG. 1: Comparative analysis of sequences of T. cruzi TcPRACA and TcPRACB proline racemase isoforms. A. Alignment of TcPRACA (Tc-A) and TcPRACB (Tc-B) nucleotide sequences: non coding sequences are shown in italics; trans-splicing signals are underlined and putative spliced leader acceptor sites are double-underlined; the region encoding the computer-predicted signal peptide is indicated by double-headed arrow; initiation of translation for TcPRACA and TcPRACB are shown by single-headed arrows; nucleotides shaded in light and dark grey, represent respectively silent mutations or point mutations; box, proline racemase active site; UUA triplets are underlined in bold and precede polyadenylation sites that are double-underlined. B. Schematic representation of amino acid sequence alignments of T. cruzi TcPRACA (Tc-A), TcPRACB (Tc-B) proline racemases. The common scale is in amino acid residue positions along the linear alignment and represent the initiation codons for TcPRACA and TcPRACB proteins, respectively; ∇ represents an alternative TcPRACA putative initiation codon; Amino acid differences are indicated above and below the vertical lines and their positions in the sequence are shown in parenthesis. SP: signal peptide; the N-terminal domain of TcPRACA extends from positions 1 to 69; SPCGT; conserved active sites of TcPRACA and TcPRACB proline racemases; N-terminus and C-terminus are indicated for both proteins. C. Hydrophobicity profile of TcPRACA: dotted line depicts the cleavage site as predicted by Von Heijne's method (aa 31-32). D. Ethidium bromide-stained gel of chromosomal bands of T. cruzi CL Brener clone after separation by PFGE (lane 1) and Southern blot hybridization with TcPRAC probe (lane 2). The sizes (Mb) of chromosomal bands are indicated, as well as the region chromosome numbers in roman numerals.

FIG. 2: Biochemical characterization of T. cruzi proline racemase isoforms and substrate specificities. A. SDS-PAGE analysis of purified rTcPRACA (lane 1) and rTcPRACB (lane 2) recombinant proteins. A 8% polyacrylamide gel was stained with Coomassie blue. Right margin, molecular weights. B. Percent of racemization of L-proline, D-proline, L-hydroxy (OH) proline and D-hydroxy (OH) proline substrates by rTcPRACB (open bar) as compared to rTcPRACA (closed bar). Racemase activity was determined with 0.25 μM of each isoform of proline racemase and 40 mM substrate in sodium acetate buffer pH 6.0. C. Percent of racemization as a function of pH: Racemase assays were performed in buffer containing 0.2 M Tris-HCl (squares), sodium acetate (triangles) and sodium phosphate (circles), 40 mM L-proline and 0.25 μM of purified rTcPRACA (closed symbols) and rTcPRACB (open symbols). After 30 min at 37° C., the reaction was stopped by heat inactivation and freezing. D. 39 kDa intracellular isoform was isolated from soluble (Ese) extracts of non-infective epimastigote forms of the parasite. Western-blots of serial dilutions of the soluble suspension was compared to known amounts of rTcPRACB protein and used for protein quantitation using Quantity One® software. Racemase assays were performed in sodium acetate buffer pH6, using 40 mM L-proline and the equivalent depicted amounts of 39 kDa (ng) contained in Ese extract.

FIG. 3: Kinetic parameters of L-proline racemization catalyzed by rTcPRACA and rTcPRACB proline racemase isoforms. The progress of racemization reaction was monitored polarimetrically, as previously described (13). A. The determination of the linear part of the curve was performed at 37° C. in medium containing 0.2 M sodium acetate, pH 6.0; 0.25 μM purified enzyme and 40 mM L-proline. rTcPRACA reactions are represented by black squares and rTcPRACB reactions by white squares. B. Initial rate of racemase activity was assayed at 37° C. in medium containing 0.2 M sodium acetate, pH 6.0, 0.125 μM of rTcPRACA (solid squares) or rTcPRACB (open squares) purified enzymes and different concentrations of L-proline. Lineweaver-Burk double reciprocal plots were used to determine values for K_{M} and Vₘₐₓ where 1/V is plotted in function of 1/[S] and the slope of the curve represents K_{M}/Vₘₐₓ. Values obtained were confirmed by using the Kaleïdagraph® program and Michaelis-Menten equation. The values are representative of six experiments with different enzyme preparations. C. Double reciprocal plot kinetics of 0.125 μM rTcPRACA proline racemase isoform in the presence (open) squares or absence (solid) squares of 6.7 μM PAC competitive inhibitor in function of L-proline concentration. For comparison: K_{M} reported for the proline racemase of C. sticklandii was 2.3 mM; kinetic assays using the native protein obtained from a soluble epimastigote fraction revealed a K_{M} of 10.7 mM and a K_{I} of 1.15 μM.

FIG. 4: Size exclusion chromatography of rTcPRACA protein using a Superdex 75 column. Fractions were eluted by HPLC at pH 6.0, B2 and B4 peaks correspond to rTcPRACA dimer and monomer species respectively. B1 and B5 eluted fractions were reloaded into the column (bold, see inserts) using the same conditions and compared to previous elution profile (not bold).

FIG. 5: Site-directed mutagenesis of TcPRACA proline racemase. Schematic representation of the active site mutagenesis of proline racemase of TcPRACA gene.

FIG. 6: Sequence alignments of proteins (Clustal X) obtained by screening SWISS-PROT and TrEMBL databases using motifs I, II and III. Amino acids involved in MI, MII and MIII are shaded in dark grey and light grey figures the 13-14 unspecific amino acids involved in M II. SWISS-PROT accession numbers of the sequences are in Table IV.

FIG. 7: Cladogram of protein sequences obtained by T-coffee alignment radial tree. See Table IV for SWISS-PROT protein accession numbers.

FIG. 8 shows the percent of racemisation inhibition of different L-proline concentrations (ranging from 10-40 mM) using the D-AAO (D-AA0/L-) microtest as compared to conventional detection using a polarimeter (Pol/L-).

FIG. 9 shows the comparison of D-AAO/HRP reaction using D-Proline alone or an equimolar mixture of D- and L-Proline as standard.

FIG. 10 shows optical density at 490 nm as a function of D-proline concentration under the following conditions.

FIG. 11 is a Graph obtained with the serial dilutions of D-proline, as positive reaction control Obs: OD of wells (−) average of OD obtained from blank wells.

FIG. 12 shows the loss of the enzymatic activity of proline racemase after mutagenesis of the residue Cys¹⁶⁰ or the residue Cys³³⁰.

### DETAILED DESCRIPTION OF THE INVENTION

Proline racemase catalyses the interconversion of L- and D-proline enantiomers and has to date been described in only two species. Originally found in the bacterium Clostridium sticklandii, it contains cysteine residues in the active site and does not require co-factors or other known coenzymes. The first eukaryotic amino acid (proline) racemase, after isolation and cloning of a gene from the pathogenic human parasite Trypanosoma cruzi, has been described. While this enzyme is intracellularly located in replicative non-infective forms of T. cruzi, membrane-bound and secreted forms of the enzyme are present upon differentiation of the parasite into non-dividing infective forms. The secreted isoform of proline racemase is a potent host B-cell mitogen supporting parasite evasion of specific immune responses.

Primarily it was essential to elucidate whether TcPRACB gene could encode a functional proline racemase. To answer this question, TcPRACA and TcPRACB paralogue genes were expressed in Escherichia coli and detailed studies were performed on biochemical and enzymatic characteristics of the recombinant proteins. This invention demonstrates that TcPRACB indeed encodes a functional proline racemase that exhibits slightly different kinetic parameters and biochemical characteristics when compared to TcPRACA enzyme. Enzymatic activities of the respective recombinant proteins showed that the 39 kDa intracellular isoform of proline racemase produced by TcPRACB construct is more stable and has higher rate of D/L-proline interconversion than the 45 kDa isoform produced by TcPRACA. Additionally, the dissociation constant of the enzyme-inhibitor complex (K_{I}) obtained with pyrrole-2-carboxylic acid, the specific inhibitor of proline racemases, is lower for the recombinant TcPRACB enzyme.

Moreover, this invention demonstrates that Cys³³⁰ and Cys¹⁶⁰ are key amino acids of the proline racemase active site since the activity of the enzyme is totally abolished by site-direct mutagenesis of these residues.

Also, multiple alignment of proline racemase amino acid sequences allowed the definition of protein signatures that can be used to identify putative proline racemases in other microorganisms. The significance of the presence of proline racemase in eukaryotes, particularly in T. cruzi, is discussed, as well as the consequences of this enzymatic activity in the biology and infectivity of the parasite.

This invention provides amino acid motifs, which are useful as signatures for proline racemaces. These amino acid motifs are as follows:

MOTIF I
[SEQ ID NO: 1]
[IVL][GD]XHXXG[ENM]XX[RD]X[VI]XG
MOTIF II
[SEQ ID NO: 2]
[NSM][VA][EP][AS][FY]X(13,14)[GK]X[IVL]XXD[IV][AS]
[YWF]GGX[FWY]
MOTIF III
[SEQ ID NO: 3]
DRSPXGXGXXAXXA
MOTIF III*
[SEQ ID NO: 4]
DRSPCGXGXXAXXA

where X is an amino acid in each of these sequences.

This invention also provides polynucleotides encoding amino acid motifs, which are also referred to herein as the “polynucleotides of the invention” and the “polypeptides of the invention.”

Databases were screened using these polynucleotide or polypeptide sequences of TcPRACA. Motifs I to III were searched. M I corresponds to [IVL][GD]XHXXG[ENM]XX[RD]X[VI]XXG, M II to of [NSM][VA][EP][AS][FY]X(13,14)[GK]X[IVL]XXD[IV][AS][YWF]GGX[FWY] M III to DRSPXGXGXXAXXA and M III* to DRSPCGXGXX AXXA. Sequences presented in the annex, where the conserved regions of 2 Cysteine residues of the active site are squared, are presented in Table V in bold with corresponding Accession numbers. The two cysteine residues are Cys³³⁰ and its homologue Cys¹⁶⁰, where residue Cys¹⁶⁰ mutation by a serine by site directed mutagenesis also induces a drastic loss of the enzymatic activity as for residue Cys³³⁰.

Proline racemase, an enzyme previously only described in protobacterium Clostridium sticklandii (11), was shown to be encoded also by the eukaryote Trypanosoma cruzi, a highly pathogenic protozoan parasite (13). The Trypanosoma cruzi proline racemase (TcPRAC), formerly called TcPA45, is an efficient mitogen for host B cells and is secreted by the metacyclic forms of the parasite upon infection, contributing to its immune-evasion and persistence through non-specific polyclonal lymphocyte activation (13). Previous results suggested that TcPRAC is encoded by two paralogous genes per haploid genome. Protein localization studies have also indicated that T. cruzi can differentially express intracellular and secreted versions of TcPRAC during cell cycle and differentiation, as the protein is found in the cytoplasm of non-infective replicative (epimastigote) forms of the parasite, and bound to the membrane or secreted in the infective, non-replicative (metacyclic trypomastigote) parasites (13).

This invention characterizes the two TcPRAC paralogues and demonstrates that both TcPRACA and TcPRACB give rise to functional isoforms of co-factor independent proline racemases, which display different biochemical properties that may well have important implications in the efficiency of the respective enzymatic activities. As suggested before by biochemical and theoretical studies for the bacterial proline racemase (11,17,18), TcPRAC activities rely on two monomeric enzyme subunits that perform interconversion of L- and/or D-proline enantiomers by a two base mechanism reaction in which the enzyme removes an α-hydrogen from the substrate and donates a proton to the opposite side of the α-carbon. It has been predicted that each subunit of the homodimer furnishes one of the sulphydryl groups (18).

The present invention demonstrates that TcPRAC enzymatic activities are bona fide dependent on the Cys³³⁰ residue of the active site, as site-specific ³³⁰Cys>Ser mutation totally abrogates L- and D-proline racemization, in agreement with a previous demonstration that TcPRAC enzymatic activity is abolished through alkylation with iodoacetate or iodoacetamide (13), similarly to the Clostridium proline racemase, where carboxymethylation was shown to occur specifically with the two cysteines of the reactive site leading to enzyme inactivation (12). The present invention demonstrates also that the residue Cys¹⁶⁰ is also a critical residue of the active site and that TcPRAC possesses two active sites in its homodimer. These observations make it possible to search for inhibitors by means of assays based on the native and mutated sequences.

While gene sequence analysis predicted that, by a mechanism of alternative splicing, TcPRACA could generate both intracellular and secreted versions of parasite proline racemase, the present invention demonstrates that TcPRACB gene sequence per se codes for a protein lacking the amino acids involved in peptide signal formation and an extra N-terminal domain present in TcPRACA protein, resembling more closely the CsPR. Thus, TcPRACB can only generate an intracellular version of TcPRAC proline racemase. This discovery makes it possible to carry out a search of one putative inhibitor of an intracellular enzyme should penetrate the cell.

Interestingly, the presence of two homologous copies of TcPRAC genes in the T. cruzi genome, coding for two similar polypeptides but with distinct specific biochemical properties, could reflect an evolutionary mechanism of gene duplication and a parasite strategy to ensure a better environmental flexibility. This assumption is comforted by the potential of TcPRACA gene to generate two related protein isoforms by alternative splicing, a mechanism that is particularly adept for cells that must respond rapidly to environmental stimuli. Primarily, trans-splicing appears indeed to be an ancient process that may constitute a selective advantage for split genes in higher organisms (19) and alternative trans-splicing was only recently proven to occur in T. cruzi (20). As an alternative for promoter selection, the regulated production of intracellular and/or secreted isoforms of proline racemase in T. cruzi by alternative trans-splicing of TcPRACA gene would allow the stringent conservation of a constant protein domain and/or the possibility of acquisition of an additional secretory region domain. As a matter of fact, recent investigations using RT-PCR based strategy and a common 3′ probe to TcPRACA and TcPRACB sequences combined to a 5′ spliced leader oligonucleotide followed by cloning and sequencing of the resulting fragments have indeed proved that an intracellular version of TcPRAC may also originate from the TcPRACA gene, corroborating this hypothesis.

Gene duplication is a relatively common event in T. cruzi that adds complexity to parasite genomic studies. Moreover, TcPRAC chromosomal mapping revealed two chromosomal bands that possess more than 3 chromosomes each and that may indicate that proline racemase genes are mapped in size-polymorphic homologous chromosomes, an important finding for proline racemase gene family characterization. Preliminary results have, for instance, revealed that T. cruzi DM28c type I strain maps proline racemase genes to the same chromoblot regions identified with T. cruzi CL type II strain used in the present invention.

It is well known that proline constitutes an important source of energy for several organisms, such as several hemoflagellates (21),(22),(23), and for flight muscles in insects (24). Furthermore, a proline oxidase system was suggested in trypanosomes (25) and the studies reporting the abundance of proline in triatominae guts (26) have implicated proline in metabolic pathways of Trypanosoma cruzi parasites as well as in its differentiation in the digestive tract of the insect vector (27). Thus, it is well accepted that T. cruzi can use L-proline as a principal source of carbon (25).

Moreover, preliminary results using parasites cultured in defined media indicate that both epimastigotes, found in the vector, and infective metacyclic trypomastigote forms can efficiently metabolize L- or D-proline as the sole source of carbon. While certain reports indicate that biosynthesis of proline occurs in trypanosomes, i.e. via reduction of glutamate carbon chains or transamination reactions, an additional and direct physiological regulation of proline might exist in the parasite to control amino acid oxidation and its subsequent degradation or yet to allow proline utilization. In fact, a recent report showed two active proline transporter systems in T. cruzi (28). T. cruzi proline racemase may possibly play a consequential role in the regulation of intracellular proline metabolic pathways, or else, it could participate in mechanisms of post-translational addition of D-amino acid to polypeptide chains.

On one hand, these hypotheses would allow for an energy gain and, on the other hand, would permit the parasite to evade host responses. In this respect, it was reported that a single D-amino acid addition in the N-terminus of a protein is sufficient to confer general resistance to lytic reactions involving host proteolytic enzymes (29). The expression of proteins containing D-amino acids in the parasite membrane would benefit the parasite inside host cell lysosomes, in addition to the contribution to the initiation of polyclonal activation, as already described for polymers composed of D-enantiomers (30), (31). Although D-amino acid inclusion in T. cruzi proteins would benefit the parasite, this hypothesis remains to be proven and direct evidence is technically difficult to obtain.

It is worth noting that metacyclogenesis of epimastigotes into infective metacyclic forms involves parasite morphologic changes that include the migration of the kinetoplast, a structure that is physically linked to the parasite flagellum, and many other significant metabolic alterations that combine to confer infectivity/virulence to the parasite (13,32). Proline racemase was shown to be preferentially localized in the flagellar pocket of infective parasite forms after metacyclogenesis (13), as are many other known proteins secreted and involved in early infection (33).

It is also conceivable that parasite proline racemase may function as an early mediator for T. cruzi differentiation through intracellular modification of internalized environmental free proline, as suggested above and already observed in some bacterial systems. As an illustration, exogenous alanine has been described as playing an important role in bacterial transcriptional regulation by controlling an operon formed by genes coding for alanine racemase and a smaller subunit of bacterial dehydrogenase (34).

In bacteria, membrane alanine receptors are responsible for alanine and proline entry into the bacterial cell (35). It can then be hypothesized that the availability of proline in the insect gut milieu associated to a mechanism of environmental sensing by specific receptors in the parasite membrane would stand for parasite praline uptake and its further intracellular racemization. Proline racemase would then play a fundamental role in the regulation of parasite growth and differentiation by its participation in both metabolic energetic pathways and the expression of proteins containing D-proline, as described above, consequently conferring parasite infectivity and its ability to escape host specific responses.

Thus far, and contrasting to the intracellular isoform of TcPRAC found in epimastigote forms of T. cruzi, the ability of metacyclic and bloodstream forms of the parasite to express and secrete proline racemase may have further implications in host/parasite interaction. In fact, the parasite-secreted isoform of proline racemase participates actively in the induction of non-specific polyclonal B-cell responses upon host infection (13) and favors parasite evasion, thus ensuring its persistence in the host.

As described for other mitogens and parasite antigens (36), (37), (38), and in addition to its mitogenic property, TcPRAC could also be involved in modifications of host cell targets enabling better parasite attachment to host cell membranes in turn assuring improved infectivity. Since several reports associate accumulation of L-proline with muscular dysfunction (39) and inhibition of muscle contraction (40), the release of proline racemase by intracellular parasites could alternatively contribute to the maintenance of infection through regulation of L-proline concentration inside host cells, as proline was described as essential for the integrity of muscular cell targets. Therefore, it has recently been demonstrated that transgenic parasites hyperexpressing TcPRACA or TcPRACB genes, but not functional knock outs, are 5-10 times more infective to host target cells pointing to a critical role of praline racemases in the ongoing of the infectious process. Likewise, previous reports demonstrated that genetic inactivation of Lysteria monocytogenes alanine racemase and D-amino acid oxidase genes abolishes bacterial pathogenicity, since the presence of D-alanine is required for the synthesis of the mucopeptide component of the cell wall that protects virtually all bacteria from the external milieu (41).

Present analysis using identified critical conserved residues in TcPRAC and C. sticklandii proline racemase genes and the screening of SWISS-PROT and TrEMBL databases led to the discovery of a minimal signature for proline racemases, DRSPXGX[GA]XXAXXA, and to confirm the presence of putative proteins in at least 10 distinct organisms. Screening of unfinished genome sequences showed highly homologous proline racemase candidate genes in an additional 8 organisms, amongst which are the fungus Aspergillus fumigatus and the bacteria Bacillus anthracis and Clostridium botulinum. This is of particular interest, since racemases, but not proline racemases, are widespread in bacteria and only recently described in more complex organisms such as T. cruzi, 42,43). These findings may possibly reflect cell adaptive responses to extracellular stimuli and uncover more general mechanisms for the regulation of gene expression by D-amino acids in eukaryotes. The finding of similar genes in human and mouse genome databases using less stringent signatures for proline racemase is striking. However, the absence of the crucial amino acid cysteine in the putative active site of those predicted proteins suggests a different functionality than that of a proline racemase.

This invention shows that TcPRAC isoforms are highly stable and have the capacity to perform their activities across a large spectrum of pH. In addition, the affinity of pyrrol-carboxylic acid, a specific inhibitor of proline racemase, is higher for TcPRAC enzymes than for CsPR.

The invention also provides amino acid or nucleic acid sequences substantially similar to specific sequences disclosed herein.

The term “substantially similar” when used to define either amino acid or nucleic acid sequences means that a particular subject sequence, for example, a mutant sequence, varies from a reference sequence by one or more substitutions, deletions, or additions, the net effect of which is to retain activity. Alternatively, nucleic acid subunits and analogs are “substantially similar” to the specific DNA sequences disclosed herein if: (a) the DNA sequence is derived from a region of the invention; (b) the DNA sequence is capable of hybridization to DNA sequences of (a) and/or which encodes active molecules; or DNA sequences that are degenerate as a result of the genetic code to the DNA sequences defined in (a) or (b) and/or which encode active molecules.

In order to preserve the activity, deletions and substitutions will preferably result in homologously or conservatively substituted sequences, meaning that a given residue is replaced by a biologically similar residue. Examples of conservative substitutions include substitution of one aliphatic residue for another, such as Ile, Val, Leu, or Ala for one another, or substitution of one polar residue for another, such as between Lys and Arg; Glu and Asp; or Gln and Asn. Other such conservative substitutions, for example, substitutions of entire regions having similar hydrophobicity characteristics, are well known. When said activity is proline racemase activity, Cys³³⁰ and Cys¹⁶⁰ must be present.

The polynucleotides of the invention can be used as probes or to select nucleotide primers notably for an amplification reaction. PCR is described in the U.S. Pat. No. 4,683,202 granted to Cetus Corp. The amplified fragments can be identified by agarose or polyacrylamide gel electrophoresis, or by a capillary electrophoresis, or alternatively by a chromatography technique (gel filtration, hydrophobic chromatography, or ion exchange chromatography). The specificity of the amplification can be ensured by a molecular hybridization using as nucleic acid probes the polynucleotides of the invention, oligonucleotides that are complementary to these polynucleotides, or their amplification products themselves.

Amplified nucleotide fragments are useful as probes in hybridization reactions in order to detect the presence of one polynucleotide according to the present invention or in order to detect the presence of a gene encoding racemase activity, such as in a biological sample. This invention also provides the amplified nucleic acid fragments (“amplicons”) defined herein above. These probes and amplicons can be radioactively or non-radioactively labeled using, for example, enzymes or fluorescent compounds.

Other techniques related to nucleic acid amplification can also be used alternatively to the PCR technique. The Strand Displacement Amplification (SDA) technique (Walker et al., 1992) is an isothermal amplification technique based on the ability of a restriction enzyme to cleave one of the strands at a recognition site (which is under a hemiphosphorothioate form), and on the property of a DNA polymerase to initiate the synthesis of a new strand from the 3′ OH end generated by the restriction enzyme, and on the property of this DNA polymerase to displace the previously synthesized strand being localized downstream.

The SDA amplification technique is more easily performed than PCR (a single thermostated water bath device is necessary), and is faster than the other amplification methods. Thus, the present invention also comprises using the nucleic acid fragments according to the invention (primers) in a method of DNA or RNA amplification, such as the SDA technique.

The polynucleotides of the invention, especially the primers according to the invention, are useful as technical means for performing different target nucleic acid amplification methods, such as:

TAS (Transcription-based Amplification System), described by Kwoh et al. in 1989;

SR (Self-Sustained Sequence Replication), described by Guatelli et al. in 1990;

NASBA (Nucleic acid Sequence Based Amplification), described by Kievitis et al. in 1991; and

TMA (Transcription Mediated Amplification).

The polynucleotides of the invention, especially the primers according to the invention, are also useful as technical means for performing methods for amplification or modification of a nucleic acid used as a probe, such as:

LCR (Ligase Chain Reaction), described by Landegren et al. in 1988 and improved by Barany et al. in 1991, who employ a thermostable ligase;

RCR (Repair Chain Reaction), described by Segev et al. in 1992;

CPR (Cycling Probe Reaction), described by Duck et al. in 1990; and

Q-beta replicase reaction, described by Miele et al. in 1983 and improved by Chu et al. in 1986, Lizardi et al. in 1988, and by Burg et al. and Stone et al. in 1996.

When the target polynucleotide to be detected is RNA, for example mRNA, a reverse transcriptase enzyme can be used before the amplification reaction in order to obtain a cDNA from the RNA contained in the biological sample. The generated cDNA can be subsequently used as the nucleic acid target for the primers or the probes used in an amplification process or a detection process according to the present invention.

The oligonucleotide probes according to the present invention hybridize specifically with a DNA or RNA molecule comprising all or part of the polynucleotide of the invention under stringent conditions. As an illustrative embodiment, the stringent hybridization conditions used in order to specifically detect a polynucleotide according to the present invention are advantageously the following:

Prehybridization and hybridization are performed as follows in order to increase the probability for heterologous hybridization:
The prehybridization and hybridization are done at 50° C. in a solution containing 5×SSC and 1×Denhardt's solution.

The washings are performed as follows:
2×SSC at 60° C. 3 times during 20 minutes each.

The non-labeled polynucleotides of the invention can be directly used as probes. Nevertheless, the polynucleotides can generally be labeled with a radioactive element (³²P, ³⁵S, ³H, ¹²⁵I) or by a non-isotopic molecule (for example, biotin, acetylaminofluorene, digoxigenin, 5-bromodesoxyuridin, fluorescein) in order to generate probes that are useful for numerous applications. Examples of non-radioactive labeling of nucleic acid fragments are described in the French Patent No. FR 78 10975 or by Urdea et al. or Sanchez-Pescador et al. 1988.

Other labeling techniques can also be used, such as those described in the French patents 2 422 956 and 2 518 755. The hybridization step can be performed in different ways. A general method comprises immobilizing the nucleic acid that has been extracted from the biological sample on a substrate (nitrocellulose, nylon, polystyrene) and then incubating, in defined conditions, the target nucleic acid with the probe. Subsequent to the hybridization step, the excess amount of the specific probe is discarded, and the hybrid molecules formed are detected by an appropriate method (radioactivity, fluorescence, or enzyme activity measurement).

Advantageously, the probes according to the present invention can have structural characteristics such that they allow signal amplification, such structural characteristics being, for example, branched DNA probes as those described by Urdea et al. in 1991 or in the European Patent No. 0 225 807 (Chiron).

In another advantageous embodiment of the present invention, the probes described herein can be used as “capture probes”, and are for this purpose immobilized on a substrate in order to capture the target nucleic acid contained in a biological sample. The captured target nucleic acid is subsequently detected with a second probe, which recognizes a sequence of the target nucleic acid that is different from the sequence recognized by the capture probe.

The oligonucleotide probes according to the present invention can also be used in a detection device comprising a matrix library of probes immobilized on a substrate, the sequence of each probe of a given length being localized in a shift of one or several bases, one from the other, each probe of the matrix library thus being complementary to a distinct sequence of the target nucleic acid. Optionally, the substrate of the matrix can be a material able to act as an electron donor, the detection of the matrix positions in which hybridization has occurred being subsequently determined by an electronic device. Such matrix libraries of probes and methods of specific detection of a target nucleic acid are described in European patent application No. 0 713 016, or PCT Application No. WO 95 33846, or also PCT Application No. WO 95 11995 (Affymax Technologies), PCT Application No. WO 97 02357 (Affymetrix Inc.), and also in U.S. Pat. No. 5,202,231 (Drmanac), said patents and patent applications being herein incorporated by reference.

The present invention also pertains to recombinant plasmids containing at least a nucleic acid according to the invention. A suitable vector for the expression in bacteria, and in particular in E. coli, is pET-28 (Novagen), which allows the production of a recombinant protein containing a 6×His affinity tag. The 6×His tag is placed at the C-terminus or N-terminus of the recombinant polypeptide.

The polypeptides according to the invention can also be prepared by conventional methods of chemical synthesis, either in a homogenous solution or in solid phase. As an illustrative embodiment of such chemical polypeptide synthesis techniques, the homogenous solution technique described by Houbenweyl in 1974 may be cited.

The polypeptides of the invention are useful for the preparation of polyclonal or monoclonal antibodies that recognize the polypeptides (SEQ ID NOS: 1, 2, 3, and 4) or fragments thereof. The monoclonal antibodies can be prepared from hybridomas according to the technique described by Kohler and Milstein in 1975. The polyclonal antibodies can be prepared by immunization of a mammal, especially a mouse or a rabbit, with a polypeptide according to the invention, which is combined with an adjuvant, and then by purifying specific antibodies contained in the serum of the immunized animal on a affinity chromatography column on which has previously been immobilized the polypeptide that has been used as the antigen.

A method of detecting a racemase encoded by a nucleotide sequence containing a subsequence encoding a peptide selected from SEQ ID NOS: 1, 2, 3, or 4.

Consequently, the invention is also directed to a method for detecting specifically the presence of a polypeptide according to the invention in a biological sample. The method comprises:
a) bringing into contact the biological sample with an antibody according to the invention; and
b) detecting antigen-antibody complex formed.

Also part of the invention is a diagnostic kit for in vitro detecting the presence of a polypeptide according to the present invention in a biological sample. The kit comprises:
a polyclonal or monoclonal antibody as described above, optionally labeled; and
a reagent allowing the detection of the antigen-antibody complexes formed, wherein the reagent carries optionally a label, or being able to be recognized itself by a labeled reagent, more particularly in the case when the above-mentioned monoclonal or polyclonal antibody is not labeled by itself.

The present invention is also directed to bioinformatic searches in data banks using the whole sequences of the polypeptides using the whole sequences of the polypeptides (SEQ ID NOS: 1, 2, 3, or 4). In this case the method detects the presence of at least a subsequence encoding a peptide selected from SEQ ID NOS: 1, 2, 3, or 4 wherein the said at least subsequence is indicative of a racemase.

The invention also pertains to:

A purified polypeptide or a peptide fragment having at least 10 amino acids, which is recognized by antibodies directed against a polynucleotide or peptide sequence according to the invention.

A monoclonal or polyclonal antibody directed against a polypeptide or a peptide fragment encoded by the polynucleotide sequences according to the invention.

A method of detecting a racemase in a biological sample comprising:
a) contacting DNA or RNA of the biological sample with a primer or a probe from a polynucleotide according to the invention, which hybridizes with a nucleotide sequence;
b) amplifying the nucleotide sequence using the primer or said probe; and
c) detecting the hybridized complex formed between the primer or probe with the DNA or RNA.

A kit for detecting the presence of a racemase in a biological sample, comprises:
a) a polynucleotide primer or probe according to the invention; and
b) reagents necessary to perform a nucleic acid hybridization reaction.

An in vitro method of screening for an active molecule capable of inhibiting a racemase encoded by a nucleic acid containing a polynucleotide according to the invention, wherein the inhibiting activity of the molecule is tested on at least said racemase, comprises:
a) providing racemase containing a polypeptide according to the invention;
b) contacting the active molecule with said racemase;
c) testing the capacity of the active molecule, at various concentrations, to inhibit the activity of the racemase; and
d) choosing the active molecule that provides an inhibitory effect of at least 80% on the activity of the racemase.

The term “recombinant” as used herein means that a protein or polypeptide employed in the invention is derived from recombinant (e.g., microbial or mammalian) expression systems. “Microbial” refers to recombinant proteins or polypeptides made in bacterial or fungal (e.g., yeast) expression systems. As a product, “recombinant microbial” defines a protein or polypeptide produced in a microbial expression system, which is essentially free of native endogenous substances. Proteins or polypeptides expressed in most bacterial cultures, e.g. E. coli, will be free of glycan. Proteins or polypeptides expressed in yeast may have a glycosylation pattern different from that expressed in mammalian cells.

The polypeptide or polynucleotide of this invention can be in isolated or purified form. The terms “isolated” or “purified”, as used in the context of this specification to define the purity of protein or polypeptide compositions, means that the protein or polypeptide composition is substantially free of other proteins of natural or endogenous origin and contains less than about 1% by mass of protein contaminants residual of production processes. Such compositions, however, can contain other proteins added as stabilizers, excipients, or co-therapeutics. These properties similarly apply to polynucleotides of the invention.

The platform of the invention relates to reagents, systems and devices for performing the process of screening of D-amino acid tests.

Appropriate carriers, diluents, and adjuvants can be combined with the polypeptides and polynucleotides described herein in order to prepare the compositions of the invention. The compositions of this invention contain the polypeptides or polynucleotides together with a solid or liquid acceptable nontoxic carrier. Such carriers can be sterile liquids, such as water an oils, including those of petroleum, animal, vegetable, or synthetic origin. Examples of suitable liquids are peanut oil, soybean oil, mineral oil, sesame oil and the like. Water is a preferred carrier. Physiological solutions can also be employed as liquid carriers.

This invention will now be described with reference to the following Examples.

### Example 1

### Cloning and Automated Sequencing

Lambda phage and plasmid DNA were prepared using standard techniques and direct sequencing was accomplished with the Big dye Terminator Kit (Perkin Elmer, Montigny-le Bretonneux, France) according to the manufacturer's Instructions. Extension products were run for 7 h in an ABI 377 automated sequencer. Briefly, to obtain the full length of the TcPRAC gene, ³²P-labeled 239 bp PCR product was used as a probe to screen a T. cruzi clone CL-Brener lambs Fix II genomic library (see details in (13)). There were isolated 4 independent positive phages. Restriction analysis and Southern blot hybridization showed two types of genomic fragments, each represented by 2 phages. Complete sequence and flanking regions of representative phages for each pattern was done. Complete characterization of TcPRACA gene, representing the first phage type, was previously described in (13). Full sequence of the putative TcPRACB gene, representing the second phage type was then performed and primers internal to the sequence were used for sequencing, as described before (13).

### Example 2

### Chromoblots

Epimastigote forms T. cruzi (clone CL Brener) are maintained by weekly passage in LIT medium. Agarose (0.7%) blocks containing 1×10⁷ cultured parasites were lysed with 0.5 M EDTA/10 mM Tris/1% sarcosyl pH 8.0, digested by proteinase K and washed in 10 mM Tris/1 mM EDTA, pH 8.0. Pulsed field gel electrophoresis (PFGE) was carried out at 18° C. using the Gene Navigator apparatus (Pharmacia, Upsala, Sweden) in 0.5×TBE. Electrophoresis were performed, as described in (14). Gels were then stained with ethidium bromide, photographed, exposed to UV light (265 nm) for 5 min and further blotted under alkaline conditions to a nylon filter (HybondN+, Amersham Life Science Inc., Cleveland, USA). DNA probe, obtained by PCR amplification of TcPRACA gene with Hi-45 (5′ CTC TCC CAT GGG GCA GGA AAA GCT TCT G 3′) [SEQ ID NO:5] and Bg-45 (5′ CTG AGC TCG ACC AGA T(CA)T ACT GC 3′) [SEQ ID NO:6] oligonucleotides (as described in (13)) was labelled with αdATP³² using Megaprime DNA labelling system (Amersham). The chromoblot was hybridized overnight in 2×Denhardt's/5×SSPE/1.5% SDS at 55° C. and washed in 2×SSPE/0.1% SDS followed by 1×SSPE at 60° C. Autoradiography was obtained by overnight exposure of the chromoblot using a Phosphorimager cassette (Molecular Dynamics, UK).

### Example 3

### Plasmid Construction and Protein Purification

TcPRACA gene fragment starting at codon 30 was obtained by PCR, using Hi- and Bg-45 primers, and cloned in frame with a C-terminal six-histidine tag into the pET28b(+) expression vector (Novagen-Tebu, Le Perray en Yvelines, France). The fragment encoding for the TcPRACB consisted of a HindIII digestion of TcPRACB gene fragment obtained by similar PCR and cloned in frame with a C-terminal six-histidine tag into the pET28b(+) expression vector. Respective recombinant proteins TcPRACA and TcPRACB were produced in E. coli BL21 (DE3) (Invitrogen, Cergy Pontoise, France) and purified using Immobilized Metal Affinity Chromatography on nickel columns (Novagen-Tebu, Le Parrayen Yvelines, France) following the manufacturers instructions.

### Example 4

### Size Exclusion Chromatography

rTcPRACA and rTcPRACB proteins were purified as described here above and dialysed against PBS pH 7.4 or 0.2 M NaOAc pH 6.0 elution buffers in dialysis cassettes (Slide-A-lyzer 7K Pierce), overnight at 4° C. The final protein concentration was adjusted to 2 mg/ml and 0.5 ml of the solution were loaded onto Pharmacia Superdex 75 column (HR10×30), previously calibrated with a medium range protein calibration kit (Pharmacia). Size exclusion chromatography (SEC) was carried out using an FPLC system (AKTA Purifier, Pharmacia). Elution was performed at a constant flow rate of 0.5 ml/min, protein fractions of 0.5 ml were collected and the absorbance was monitored at 280 nm. Each fraction was assayed in racemization assays as described here below. Fractions B1 and B5, were reloaded in the Superdex 75 column and submitted to a further SEC to verify the purity of the fractions.

### Example 5

### Racemization Assays

The percent of racemization with different concentrations of L-proline, D-proline, L-hydroxy (OH)-proline, D-hydroxy (OH)-proline was calculated, as described in (13), by incubating a 500 μl mixture of 0.25 μM of dimeric protein and 40 mM substrate in 0.2 M sodium acetate pH 6.0 for 30 min or 1 h at 37° C. The reaction was stopped by incubating for 10 min at 80° C. and freezing. Water (1 ml) was then added, and the optical rotation was measured in a polarimeter 241MC (Perkin Elmer, Montigny le Bretonneux, France) at a wavelength of 365 nm, in a cell with a path length of 10 cm, at a precision of 0.001 degree. The percent of racemization of 40 mM L-proline as a function of pH was determined using 0.2 M sodium acetate, potassium phosphate and Tris-HCl buffers; reactions were incubated 30 min at 37° C., as described above. All reagents were purchased from Sigma.

### Example 6

### Kinetic Assays

Concentrations of L- and D-proline were determined polarimetrically from the optical rotation of the solution at 365 nm in a cell of 10 cm path length, thermostated at 37° C. Preliminary assays were done with 40 mM of L-proline in 0.2 M sodium acetate pH 6 in a final volume of 1.5 ml. Optical rotation was measured every 5 sec during 10 min and every 5 min to 1 hour. After determination of the linear part of the curve, velocity in 5-160 mM substrate was measured every 30 sec during 10 min to determine K_{M} and Vₘₐₓ. Calculations were done using the Kaleïdagraph program. Inhibition assays were done by incubating 0.125 μM dimeric protein, 6.7 μM-6 mM pyrrole-2-carboxylic acid (PAC), 20 to 160 mM L-proline, as described above. Graphic representation and linear curve regression allowed the determination of K_{I} as [PAC]/[(slope with PAC/slope without PAC)−1]. All reagents were purchased from Sigma.

### Example 7

### Site-Directed Mutagenesis of C^{330S}TcPRACA

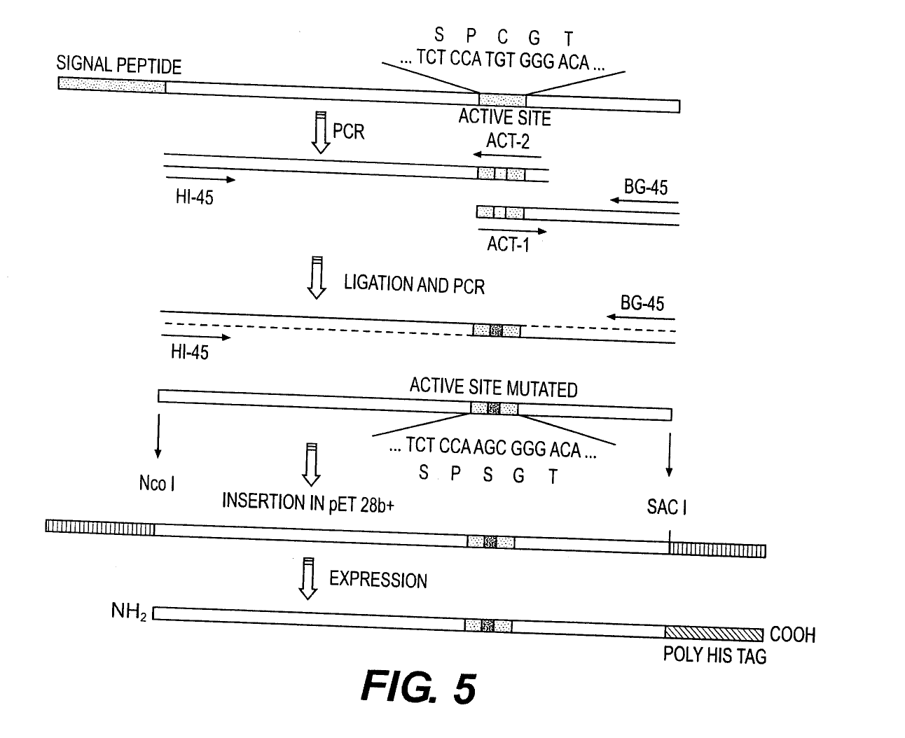

Site-directed mutagenesis was performed by PCR, adapting the method of Higuchi et al. (15). Briefly, mutation of Cys³³⁰ of the proline racemase active site was produced by two successive polymerase chain reactions based on site-directed mutagenesis using two overlapping mutagenic primers: (act-1) 5′ GCG GAT CGC TCT CCA AGC GGG ACA GGC ACC 3′ [SEQ ID NO:7] and (act-2) 5′ GGT GCC TGT CCC GCT TGG AGA GCG ATC CGC 3′, [SEQ ID NO:8] designed to introduce a single codon mutation in the active site by replacement of the cysteine (TGT) at the position 330 by a serin (AGC). A first step standard PCR amplification was performed using the TcPRACA DNA as template and a mixture of act-1 primer and the reverse C-terminus primer (Bg-45) 5′ CTG AGC TCG ACC AGA T(CA)T ACT GC 3′ (codon 423), or a mixture of act-2 primer and the forward N-terminus primer (Hi-45) 5′ CTC TCC CAT GGG GCA GGA AAA GCT TCT G 3′ (codon −53) (see FIG. 5). Resulting amplified fragments of, respectively, 316 bp and 918 bp were purified by Qiagen PCR extraction kit (Qiagen, Courtaboeuf, France), as prescribed, and further ligated by T4 ligase to generate a template consisting of the full length of a potentially mutated TcPRACA* coding sequence used for the second step PCR. Amplification of this template was performed using forward Hi-45 and reverse Bg-45 primers and the resulting TcPRACA* fragment encoding for the mature proline racemase was purified and cloned in pCR®2.1-TOPO® vector (Invitrogen). TOP10 competent E. coli were transformed with the pCR®2.1-TOP®-TcPRACA* construct and plasmid DNA isolated from individual clones prepared for DNA sequencing. Positive mutants were then sub-cloned in frame with a C-terminal six-histidine tag into the Nco I/Sac I sites of the pET 28b(+) expression vector (Novagen-Tebu, Le Parrayen Yvelines, France). Sub-clones of pET28b(+)-TcPRACA* produced in E. coli (DH5a) were sequenced again to confirm the presence of the mutation. Soluble recombinant C^{330S}TcPRACA protein was produced in E. coli BL21(DE3) (Invitrogen) and purified using a nickel column, (Novagen-Tebu), according the manufacturer's instructions.

### Example 8

### Mutagenesis

To verify the implication of the residue Cys160 in the reaction mechanism of the proline racemase, a site specific mutagenesis was performed to replace the residue Cys160 by a Serine, similarly to mutation described for Cys330 residue (see Example 7). Briefly, the site specific mutagenesis was performed by PCR using the following primers:

Ser160-Forward: ^{5′}GGCTATTTAAATATGTCTGGACATAACTCAATTGCAGCG^{3′}

### Ser160-Reverse: ^{5′}CGCTGCAATTGAGTTATGTCCAGACATATTTAAATAGC^{3′}

The presence of the mutation Cystein-Serine was verified by sequencing of the respective plasmids containing the PCR products, as shown here below. The plasmid pET-C160S was used to transform E. coli BL21(DE3) and to produce the corresponding recombinant mutated protein.

| | | | |
|---|---|---|---|
| | 139 | M D T C G Y L N M C G H N G I A A | 145 |
| pET-TcPRAC | 499 | ATCGATACCGCTGGCTATTTAAATATGTGTGGACATAACTCAATTGCAGCG | 550 |
| Ser160-F/R | | GGCTATTTAAATATGTCTGGACATAACTCAATTGCAGCG | 550 |
| pET-C160S | 499 | ATGGATACCGGTGGCTATTTAAATATGTCTGGACATAACTCAATTGCAGCG | 550 |
| pET-C330S | 499 | ATGGATACCGGTGGCTATTTAAATATGTGTGGACATAACTCAATTGCAGCG | 550 |
| | 139 | M D T C G Y L N M S G H N G I A A | 145 |
| | 318 | V I F G N R Q A D R S P C G T C T | 334 |
| pET-TcPRAC | 999 | GTGATATTTGGCAATCGCCAGGCGGATCGCTCTCCATGTGGGACAGGCACC | 1050 |
| Ser330-F/R | | GCGGATCGCTCTCCAAGCGGGACAGGCACC | 1050 |
| pET-C160S | 999 | GTGATATTTGGCAATCGCCAGGCGGATCGCTCTCCATGTGGGACAGGCACC | 1050 |
| pET-C330S | 999 | GTGATATTTGGCAATCGCCAGGCGGATCGCTCTCCAAGCGGGACAGGCACC | 1050 |
| | 318 | V I F G N R Q A D R S P S G T C T | 334 |

Underlined are the primer sequences used for the site specific mutageneses. The mutations Cys→Ser are represented in bold and underlined for both Cys160 and Cys330 residues.

### Example 9

### Expression of a Functional Intracellular Isoform of Proline Racemase

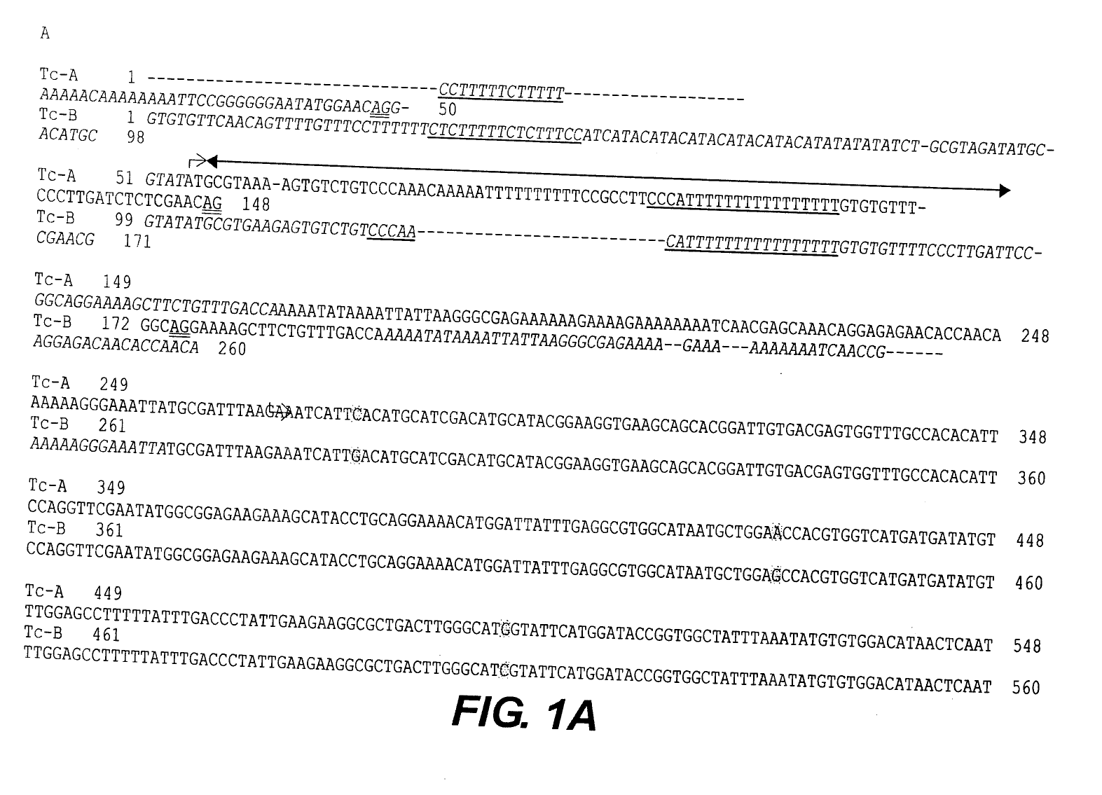

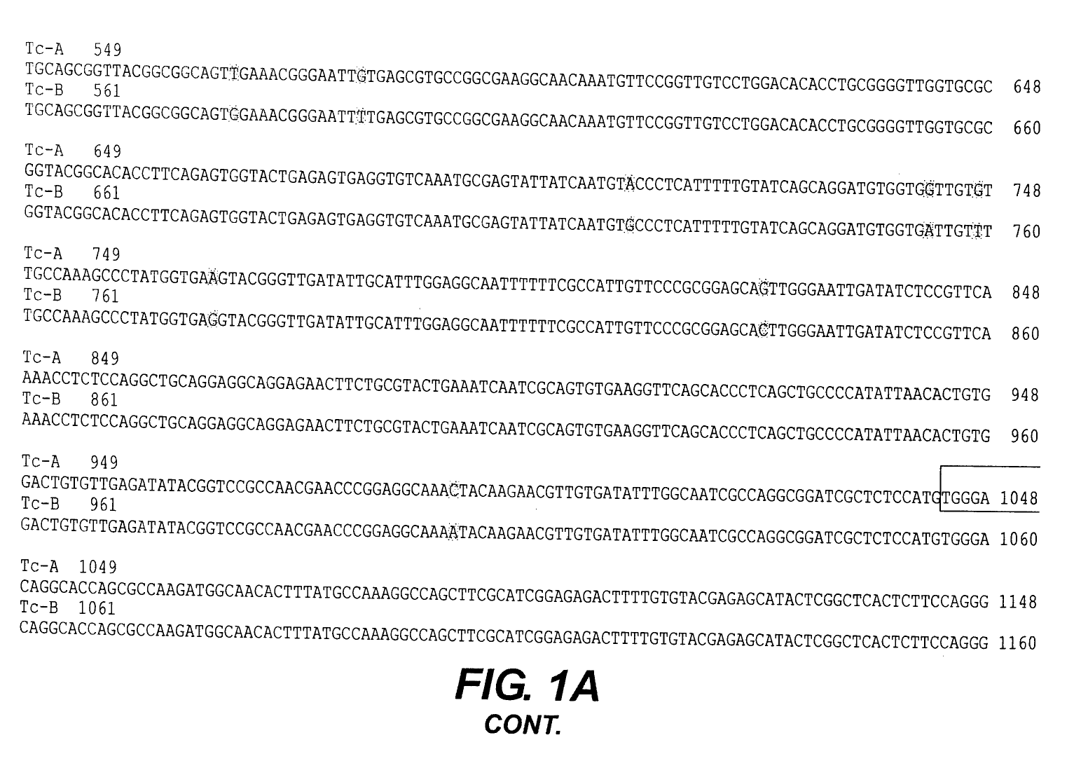

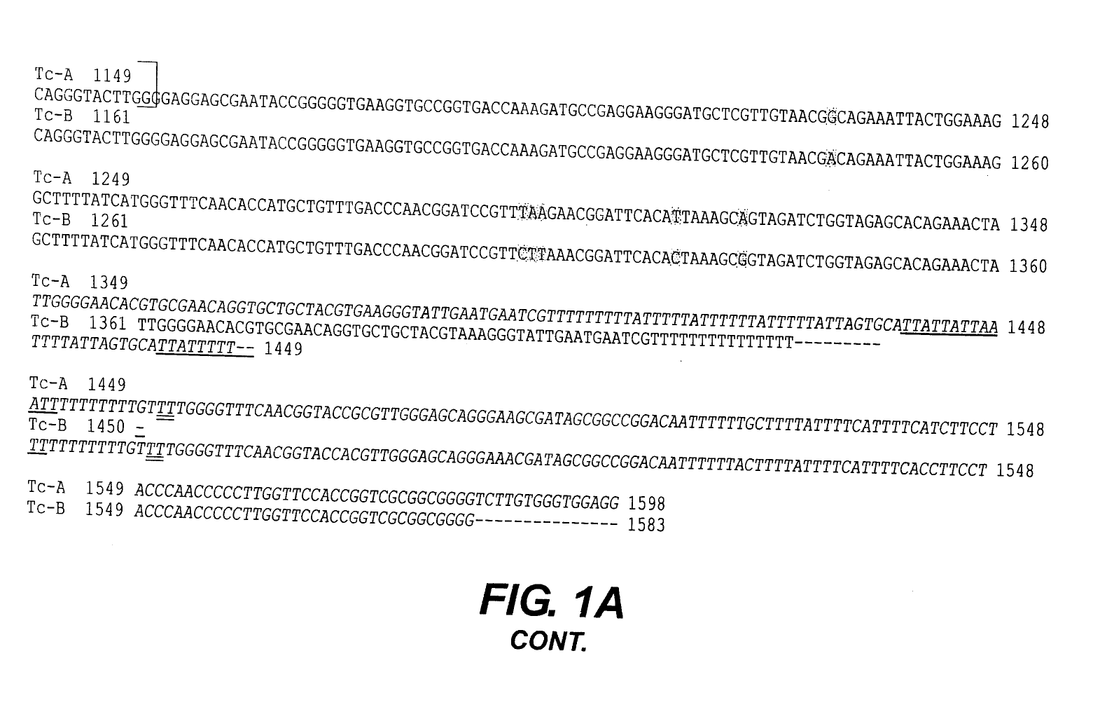

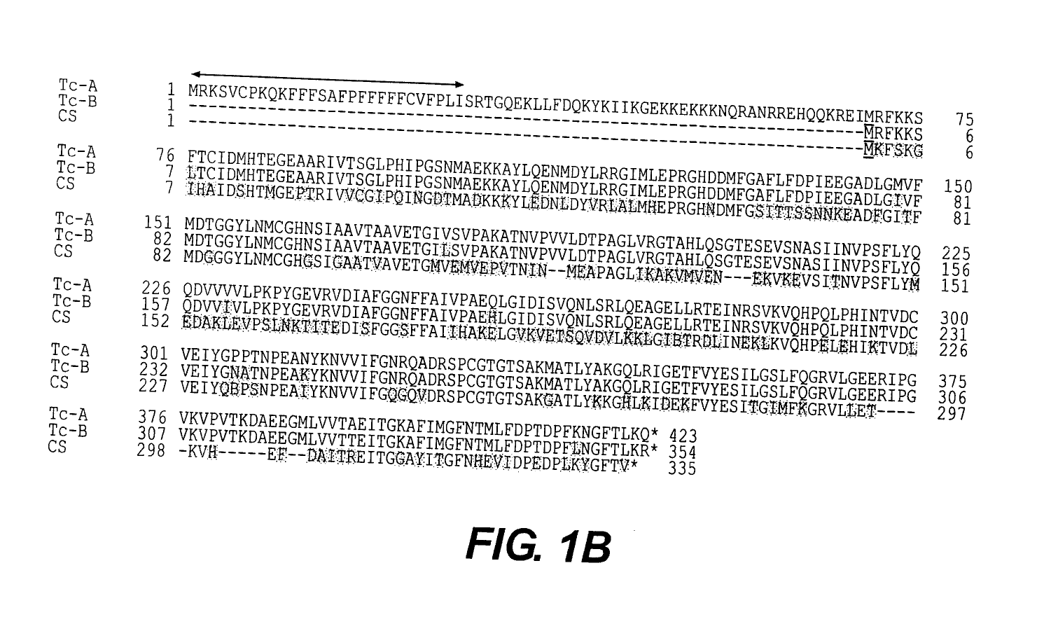

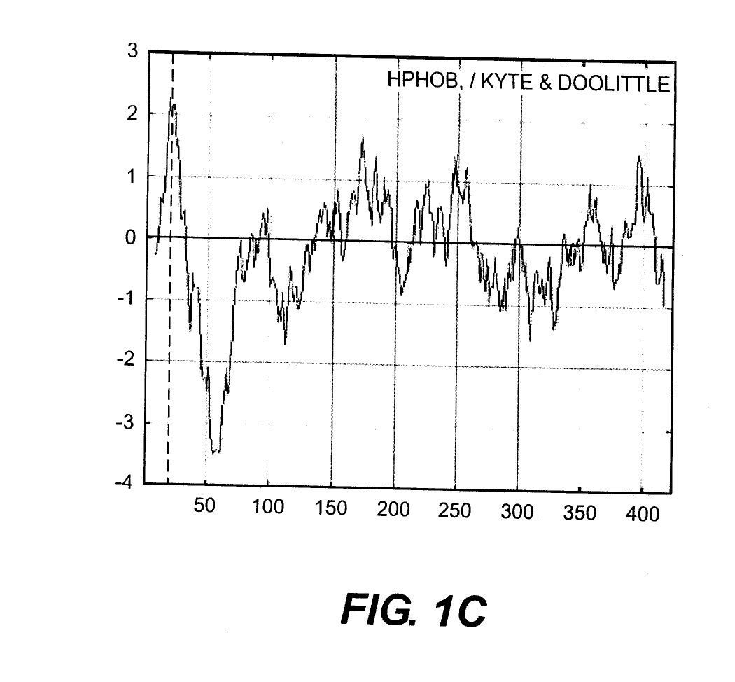

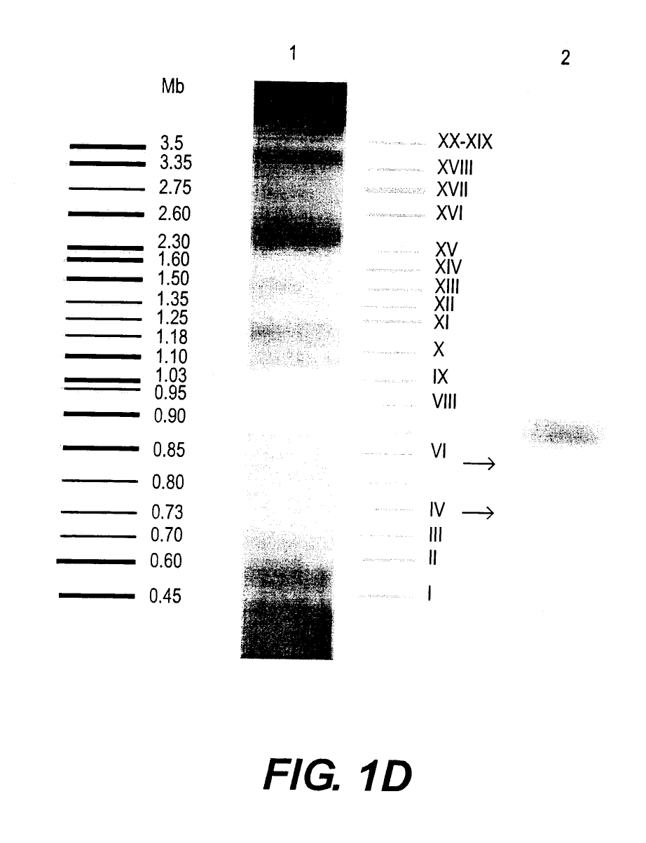

Previously characterized was a TcPRAC gene from T. cruzi, and it was demonstrated in vivo and in vitro that it encodes a proline racemase enzyme (13). Analysis of the genomic organization and transcription of the TcPRAC gene indicated the presence of two paralogue gene copies per haploid genome, named TcPRACA¹ and TcPRACB². It was shown that TcPRACA encodes a functional co-factor independent proline racemase, closely resembling the C. sticklandii proline racemase (CsPR) (11). Now sequenced was the full length of TcPRACB and, as can be observed in FIG. 1A, TcPRACA and TcPRACB genes both possess the characteristic trypanosome polypyrimidine-rich motifs in the intergenic region that are crucial trans-splicing signals when located upstream of an (AG)-dinucleotide used as acceptor site. As in other T. cruzi genes, UUA triplets are found at the end of the 3′ untranslated region preceding the polyadenylation site. Comparison between the two sequences revealed 14 point mutations (resulting in 96% identity) giving rise to 7 amino acid differences. When expressed, the TcPRACB is predicted to produce a shorter protein (39 kDa) whose translation would start at the ATG codon at position 274 located downstream of the (AG)-spliced leader acceptor site (at position 175). In comparison, TcPRACA has an open reading frame that encodes a peptide with an apparent molecular mass of 45 kDa. The schematic protein sequence alignment of the two proteins TcPRACA and TcPRACB depicted in FIG. 1B reveals that TcPRACB proline racemase lacks the amino acid sequence corresponding to the signal peptide observed in the TcPRACA protein (hatched box in the figure; see predicted cleavage site in FIG. 1C). Therefore the TcPRACB would produce a 39 kDa, intracellular and non-secreted isoform of the protein. As with CsPR (11) and TcPRACA (13 and FIG. 1B), the active site of proline racemase is conserved in TcPRACB sequence. Furthermore, while differing by only 7 amino acids, both the TcPRACA and TcPRACB sequences display around 50% homology to the CsPR (13). In accordance with other protein-coding genes in T. cruzi, TcPRAC genes are located on two different chromosomal bands of which one contains three or more chromosomes of similar size, see FIG. 1D. Thus, hybridization of blots containing T. cruzi CL Brener chromosomal bands separated by pulsed field gel electrophoresis revealed that sequences recognized by an homologous probe to both TcPRACA and TcPRACB are mapped in neighboring migrating bands of approximately 0.9 Mb and 0.8 Mb, corresponding respectively to regions VII and V, according to Cano et al. numbering system (14).

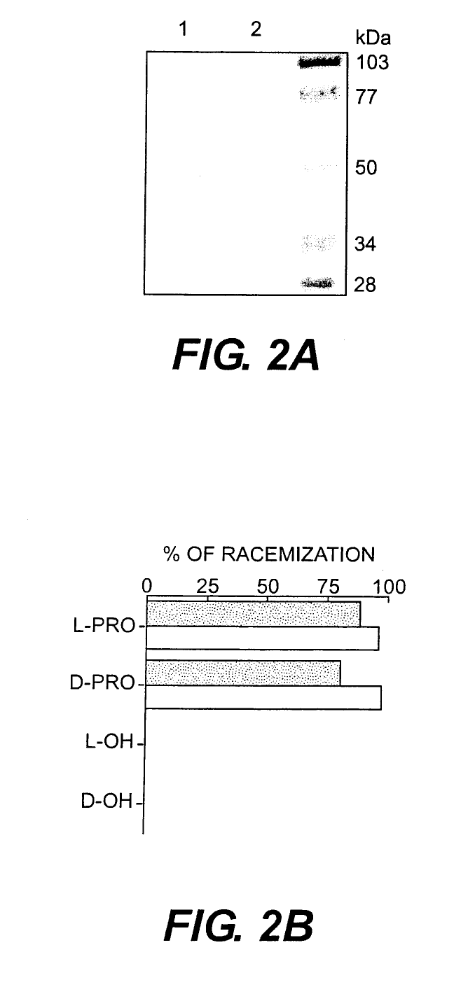

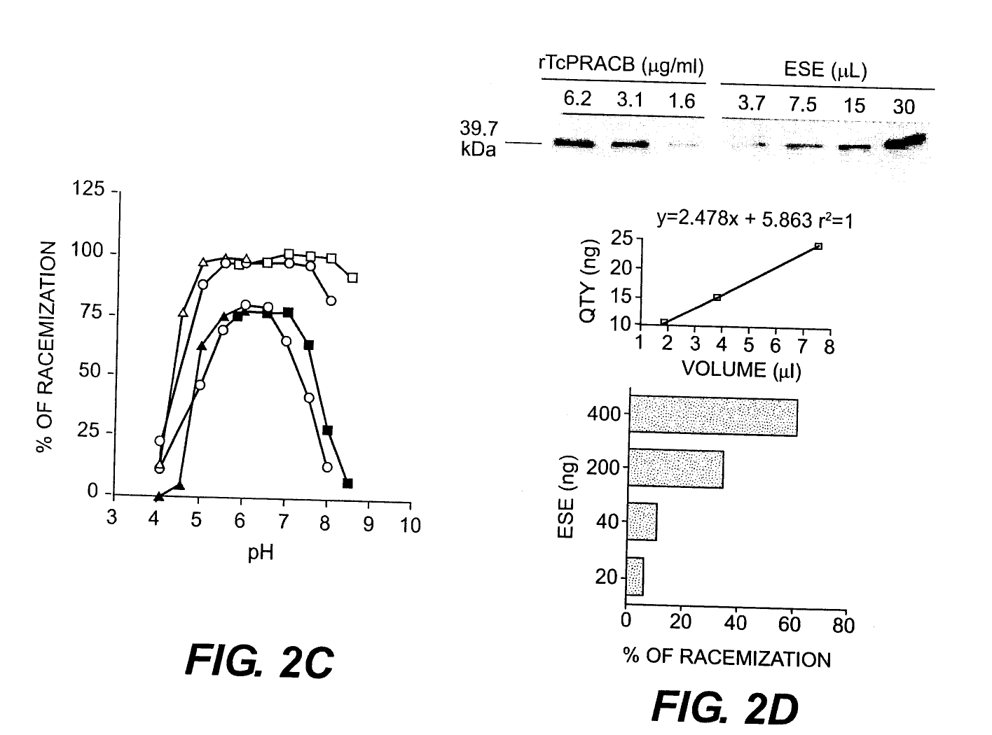

In order to verify if the TcPRACB gene could encode a functional proline racemase, both T. cruzi paralogues were expressed in E. coli to produce C-terminal His₆-tagged recombinant proteins. After purification by affinity chromatography on nickel-nitrilotriacetic acid agarose column, recombinant proteins were separated by SDS gel electrophoresis revealing single bands with the expected sizes of 45.8 and 40.1 kDa, respectively, for the rTcPRACA and rTcPRACB proteins (FIG. 2A). To determine whether rTcPRACB displays proline racemase enzymatic activity, biochemical assays were employed to measure the shift in optical rotation of L- and D-proline substrates, as described (13). As can be seen in FIG. 2B, rTcPRACB racemizes both L- and D-proline but not L-hydroxy-proline, like rTcPRACA. In a similar manner, rTcPRACB is a co-factor independent proline racemase as described for CsPR (11) and rTcPRACA (13) proline racemases. The rate of conversion of L- into D-proline was measured at various pH values using both recombinant enzymes. As illustrated in FIG. 2C, rTcPRACA activity clearly shows a pH dependency with an optimal activity from pH 5.5 to 7.0. In contrast, the optimum activity of rTcPRACB can be observed in a large pH spectrum varying from pH 4.5 to 8.5. These results revealed that translation of the open reading frame of both TcPRAC genes copies result in functional proline racemase isoforms. As previously described, Western blot analysis of non-infective epimastigote parasite extracts using antibodies raised against the 45 kDa secreted proline racemase had previously revealed a 39 kDa protein mostly in the soluble cellular fraction, only weakly in the cellular insoluble fraction and absent from culture medium (13). To demonstrate that the intracellular 39 kDa isoform of the protein was equally functional in vivo, soluble cellular extracts were obtained from 5×10⁸ epimastigotes, non-infective parasites and the levels of 39 kDa soluble protein quantified by Western blot comparatively to known amounts of rTcPRACB enzyme. As can be observed in FIG. 2D, the intracellular isoform of the protein is indeed functional in vivo, since proline racemase enzymatic activity was displayed and levels of racemization were dependent on protein concentration. This discovery is useful for specific inhibitors reaching the intracellular compartment.

### Example 10

### Functional Analysis and Kinetic Properties of Recombinant T. cruzi Proline Racemases

Since the TcPRAC gene copies encode for secreted and non-secreted isoforms of proline racemase with distinct pH requirements for activity, our investigation was made to determine whether other biochemical properties differ between rTcPRACA and rTcPRACB proteins. Such differences might reflect the cellular localization of the protein during parasite differentiation and survival in the host. Both rTcPRACA and rTcPRACB enzyme activities are maximal at 37° C. and can be abolished by heating for 5 min at 80° C. However, the stability of the two recombinant enzymes differs considerably, when analyzed under different storage conditions. Thus, as shown in Table 1, purified rTcPRACB is highly stable, since its activity is maintained for at least 10 days at room temperature in 0.5 M imidazol buffer pH 8.0, as compared to rTcPRACA that loses 84% of its activity under such conditions. In contrast, most of the enzymatic activity of rTcPRACA is maintained at 4° C. (65%), compared to that of rTcPRACB (34%). Both enzymes can be preserved in 50% glycerol at −20° C., or diluted in sodium acetate buffer at pH 6.0, but under these storage conditions rTcPRACA activity is impaired. However, best preservation of both recombinant proline racemases was undoubtedly obtained when proteins were kept at −20° C. as ammonium sulfate precipitates. Preservation is important for a kit.

TABLE I
Stability of recombinant TcPRACA and TcPRACB
proline racemases under different storage conditions
% of preservation of proline racemase activity

| | Column | | | | NaOAc | | |
|---|---|---|---|---|---|---|---|
| | | | | Gly/ | pH 6 | (NH₄)₂SO₄ | |
| Protein | CTRL | RT | +4° C. | −20° C. | 4° C. | 4° C. | −20° C. |
| rTcPRACA | 100.0 | 16.0 | 66.5 | 62.9 | 31.0 | 53.9 | 100.0 |
| rTcPRACB | 100.0 | 100.0 | 34.0 | 93.6 | 77.6 | 98.4 | 100.0 |

After purification on nickel-nitrilotriacetic acid agarose column, recombinant proteins were kept for 10 days in nickel column buffer (20 mM Tris/500 mM NaCl/500 mM imidazol, pH 8.0) at room temperature (RT) or at +4° C., or either diluted in 50% glycerol and maintained at −20° C. (Gly/−20° C.) or in optimum pH buffer (NaOAc, pH 6.0) at 4° C. Recombinant enzymes were precipitated in (NH₄)₂SO₄ and kept in solution at 4° C. or pellet dried at −20° C. Racemase assays were performed for 30 min at 37° C. Percent of preservation was determined polarimetrically using 0.25 μM of either purified rTcPRACA or rTcPRACB enzymes and 40 mM of L-proline, as compared to results obtained with freshly purified proteins (CTRL). These results are representative of at least two independent experiments.

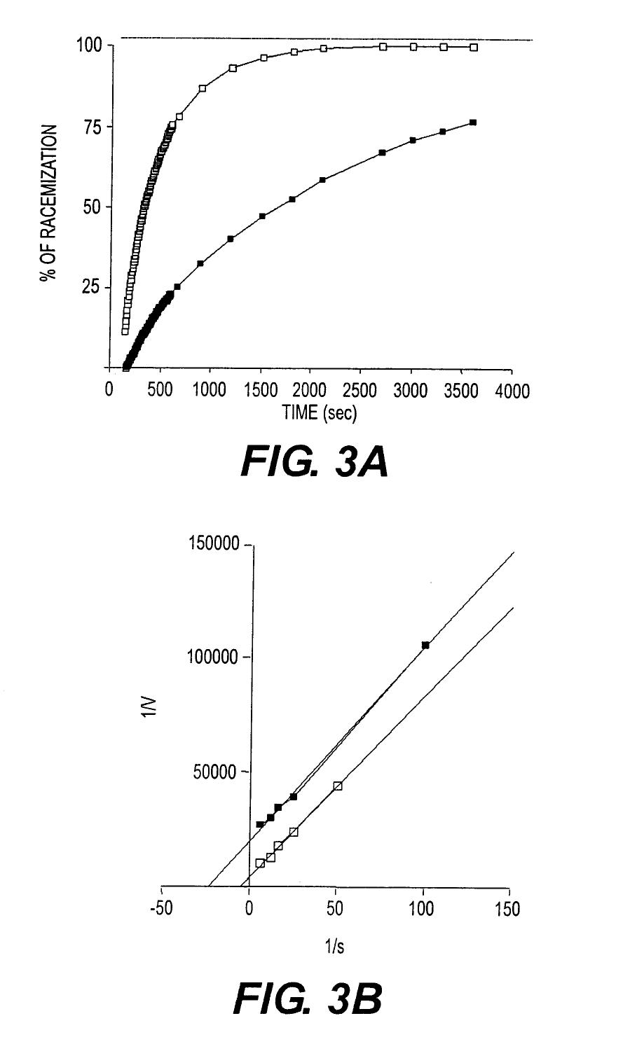

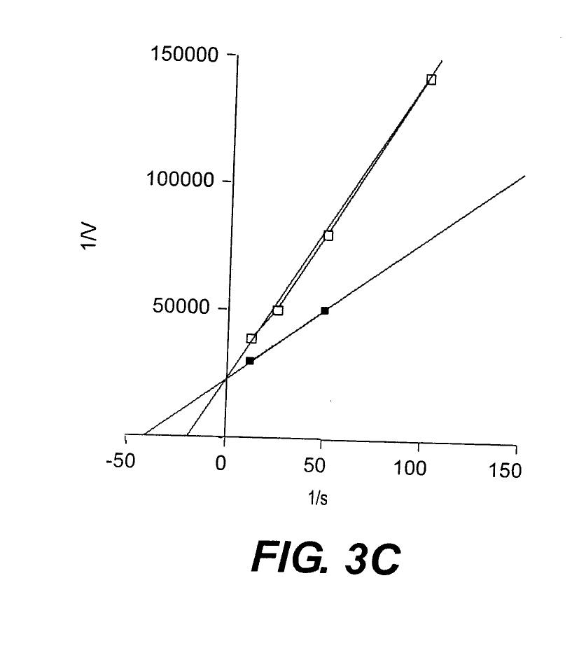

Both recombinant enzymes exhibited Michaelis-Menten kinetics (FIG. 3A) and rTcPRACB had a higher activity than rTcPRACA. Indeed, as can be observed in FIG. 3B, analysis of L>D conversion of serial dilutions of L-proline catalyzed by a constant amount of each enzyme showed that rTcPRACB enzyme (K_{M} of 75 mM and Vₘₐₓ of 2×10⁻⁴ mol·sec⁻¹) has a higher velocity as compared to rTcPRACA (K_{M} of 29 mM and Vₘₐₓ of 5.3×10 mol·sec⁻¹). In order to determine the K_{I} values for pyrrole-2-carboxylic acid (PAC), the specific and competitive inhibitor of CsPR (16), assays were performed with both recombinant proteins. These assays revealed that PAC is comparably effective as inhibitor of rTcPRACA (FIG. 3C) and rTcPRACB, and K_{I} values obtained were, respectively, 5.7 μM and 3.6 μM. The difference in K_{I} values reflects almost perfectly the difference in K_{M} values reported for both enzymes, which are similar to that of the native protein. These K_{I} values indicate that the affinity of PAC inhibitor is higher for rTcPRACA and rTcPRACB than for CsPR (K_{I} of 18 μM). The Kₘ and K_{I} values are important for an inhibitor.

### Example 11

### Requirement of a Dimeric Structure for Proline Racemase Activity

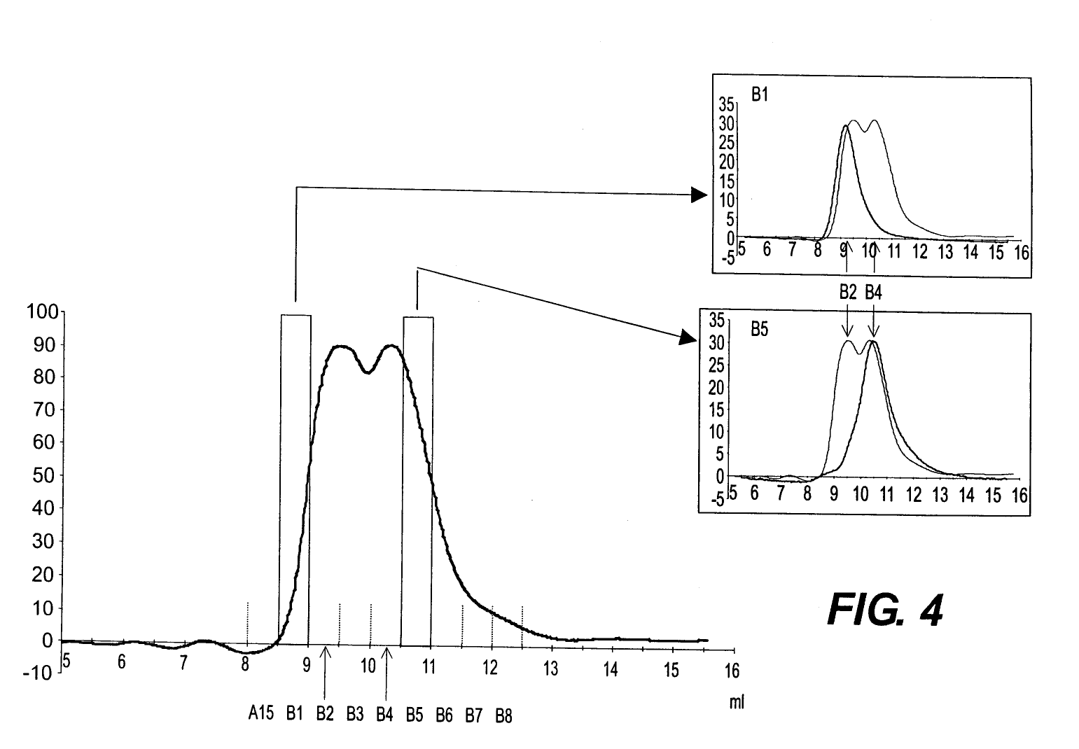

When rTcPRACA was submitted to size exclusion chromatography on a Superdex 75 column at pH 6.0, two peaks of protein were eluted, respectively, around 80 kDa (B2 fraction) and 43 kDa (B4 fraction), presumably corresponding to dimeric and monomeric forms of the enzyme (FIG. 4). Western blot analysis of whole T. cruzi epimastigote extracts using non-denaturing PAGE had previously indicated a molecular mass of 80 kDa for the native protein while a 45 kDa band was obtained by SDS-PAGE (13). In order to eliminate cross-contamination, B1 and B5 fractions, eluted, respectively, at the start and at the end of the predicted dimer (B2) or monomer (B4) peaks, were reloaded on the column and the profiles obtained (see FIG. 4 inserts) confirmed the purity of the fractions. Enzyme activity resides in the 80 kDa peak, but not in the 43 kDa peak (Table II). These results corroborated that two subunits of the protein are necessary for racemase activity. At neutral pH (7.4 or above), the rTcPRACA gives rise to high molecular weight aggregates which are not observed with rTcPRACB, consistently with its broader optimal pH spectrum. The enzyme should be in optimal pH conditions for a kit buffer, for example.

TABLE II
Racemase activity of recombinant TcPRACA
fractions after size exclusion chromatography

| Fractions | A15 | B1 | B2 | B3 | B4 | B5 | B6 | B7 |
|---|---|---|---|---|---|---|---|---|
| % racemization | 1.3 | 35.5 | 62.9 | 42.8 | 0.7 | 0 | 0 | 0 |

After elution from Superdex 75 column, 20 μl of each peak (A15 to B7, see FIG. 4) corresponding to 1 μg of protein were incubated 1 h at 37° C. with 40 mM of L-proline in 0.2 M NaOAc, pH 6.0. Optical rotation was measured and % of racemization was determined as described in Example 5.

### Example 11

### Abrogation of Proline Racemase Activity by Mutation of Cys³³⁰ and Alternately Cys¹⁶⁰ of the Catalytic Site

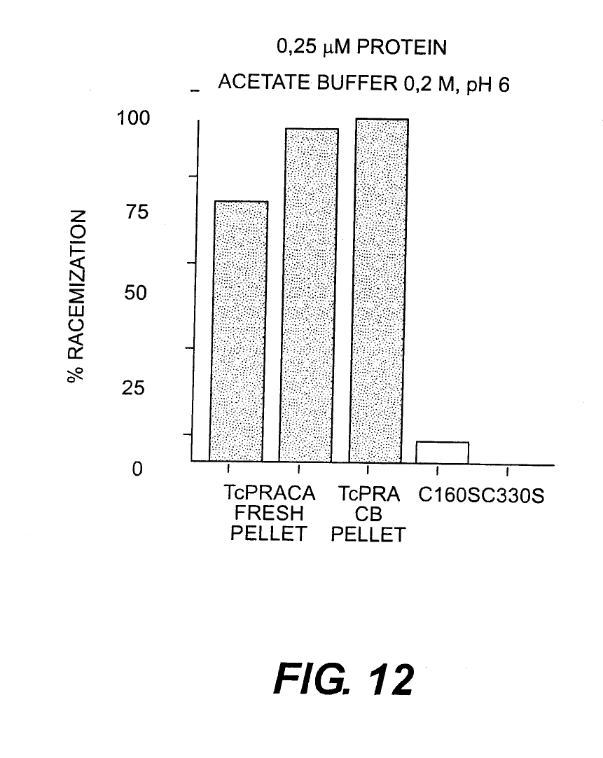

C. sticklandii proline racemase is described as a homodimeric enzyme with subunits of 38 kDa and a single proline binding site for every two subunits, where two cysteines at position 256 might play a crucial role in catalysis by the transfer of protons from and to the bound substrate (12). It has previously been shown that mitogenic properties of the T. cruzi proline racemase are dependent on the integrity of the enzyme active site, as inhibition of B-cell proliferation is obtained by substrate competition and specific use of analogues (PAC) resembling the structure assumed by the substrate proline in its transition state (16). To verify the potential role of the cysteine residues at the active site of the T. cruzi proline racemase, Cys³³⁰ and alternately Cys¹⁶⁰ were replaced by a serine residue through site specific mutation of TcPRACA. The choice of serine as the substituting amino acid was made to avoid further major disturbances on three dimensional structure of the protein (see strategy in FIG. 5 above). After confirmation of the single codon mutation through sequencing of the construct, the C^{330S} or C^{160S} rTcPRACA mutant proline racemase was expressed in E. coli and purified in the same manner as wild type rTcPRACA. Then used were C^{330S} or C^{160S} rTcPRACA in racemization assays to verify the effects of the mutation on the enzymatic activity of the protein. As can be observed in Table III (and in FIG. 12) a total loss of proline racemase activity is observed as compared to the wild type enzyme, establishing that proton transfer during proline racemization is specifically dependent on the presence of the cysteine residue in the active site.

TABLE III
Loss of racemase enzymatic activity in the site direct ^{C330S} rTcPRACA

| Data set | rTcPRACA | | | | ^{C330S} rTcPRACA | | | |
|---|---|---|---|---|---|---|---|---|
| Time (min) | 0 | 10 | 30 | 60 | 0 | 10 | 30 | 60 |
| Optical rotation | −0.385 | −0.300 | −0.162 | −0.088 | −0.385 | −0.382 | −0.391 | −0.387 |
| % racemization | 0 | 22 | 58 | 77 | 0 | 0 | 0 | 0 |

After purification, 5 μg of r TcPRACA or C^{330S}rTcPRACA were incubated at 37° C. with 40 mM of L-proline in NaOAc buffer, pH 6.0. Optical rotation was measured at different times and % of racemization was determined as described in Example 5.

### Example 12

### Proline Racemase Protein Signatures and Putative Proline Racemases in Sequence Databases

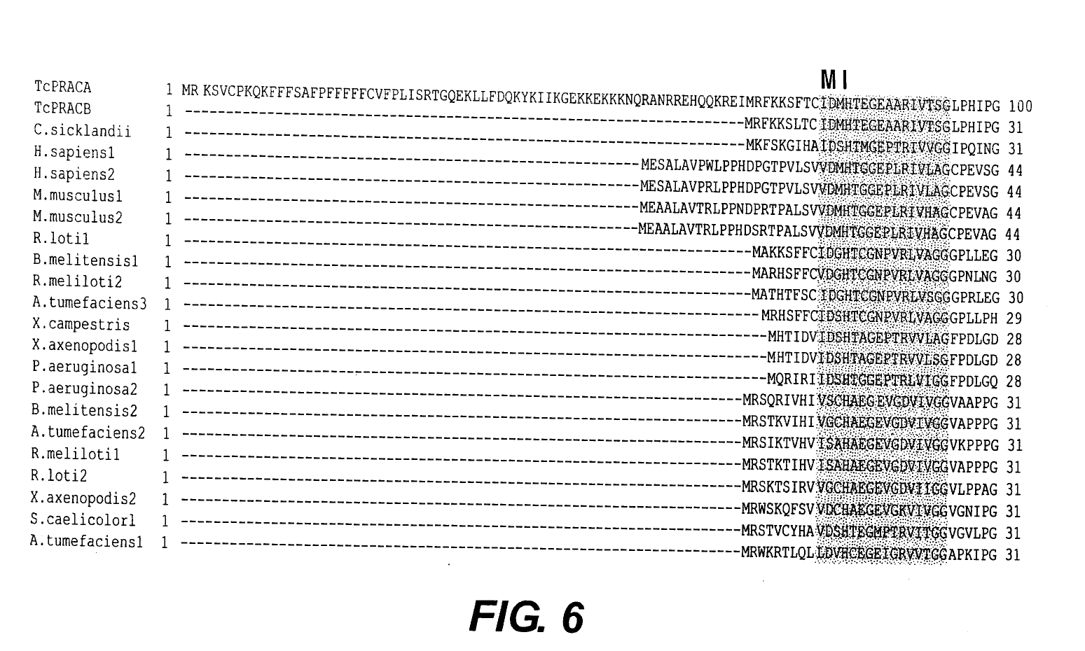

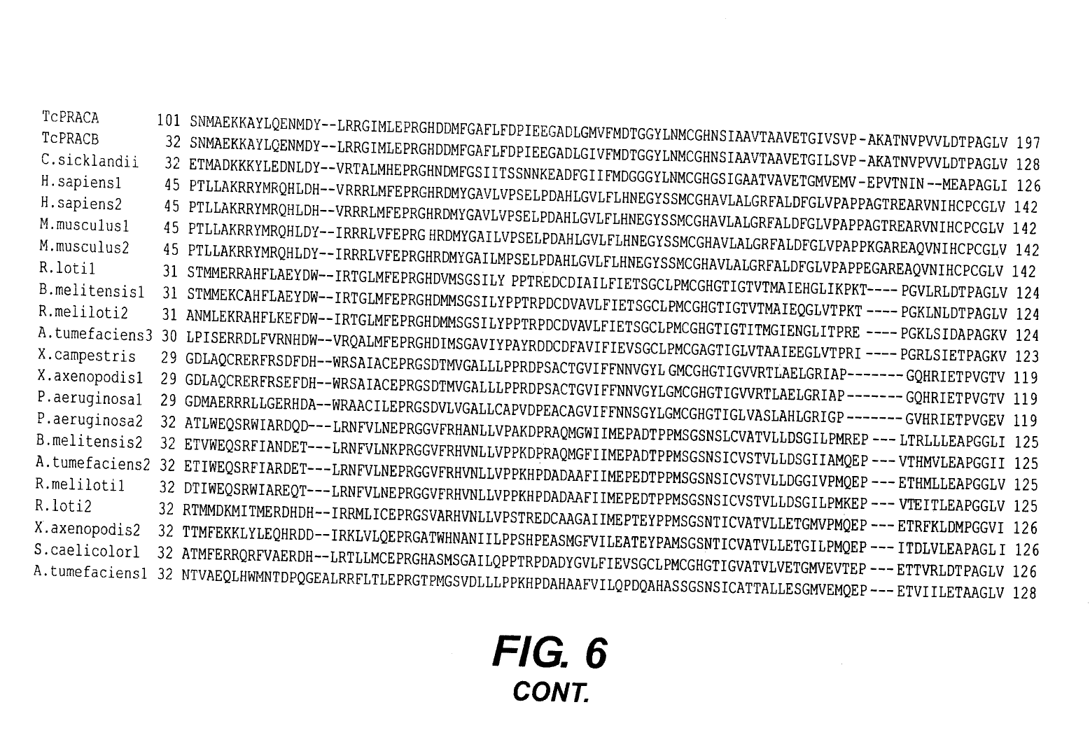

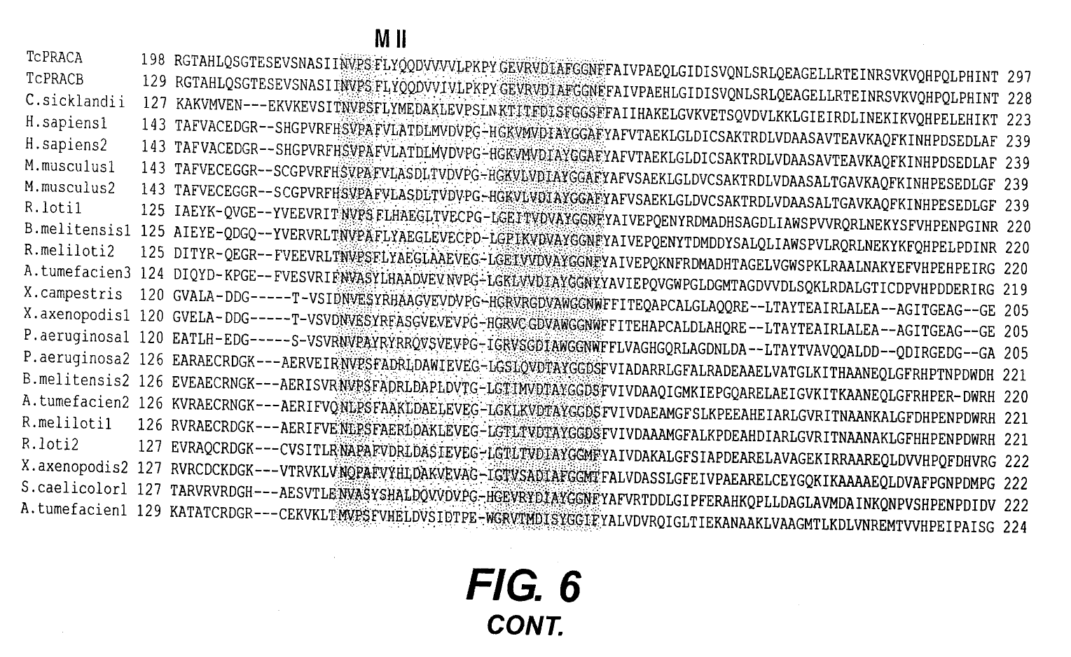

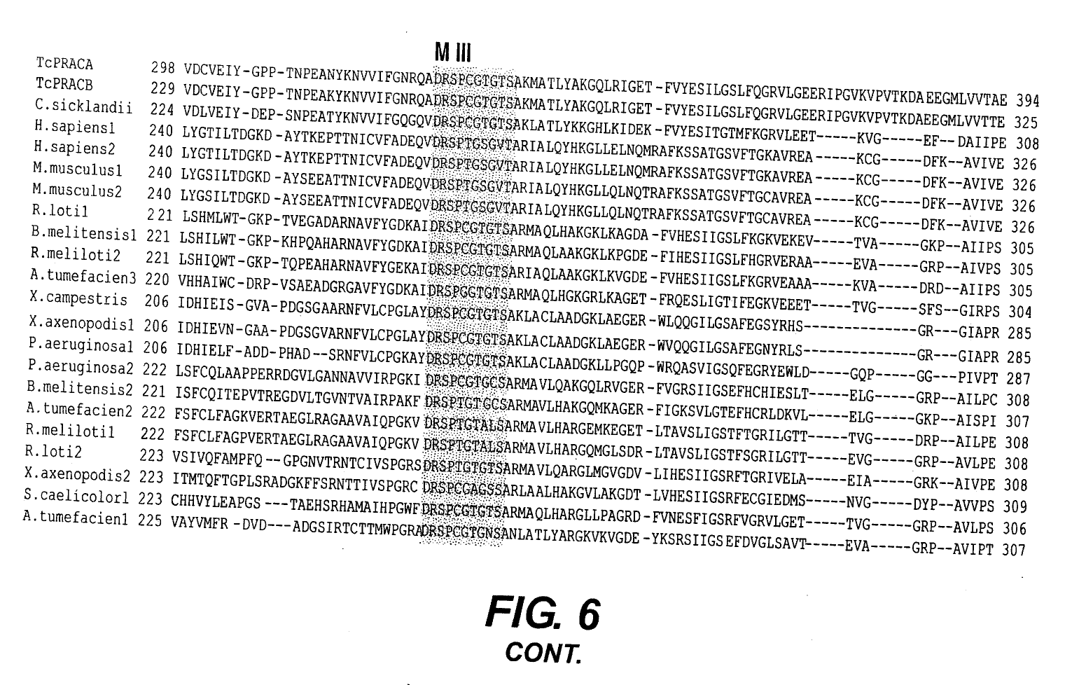

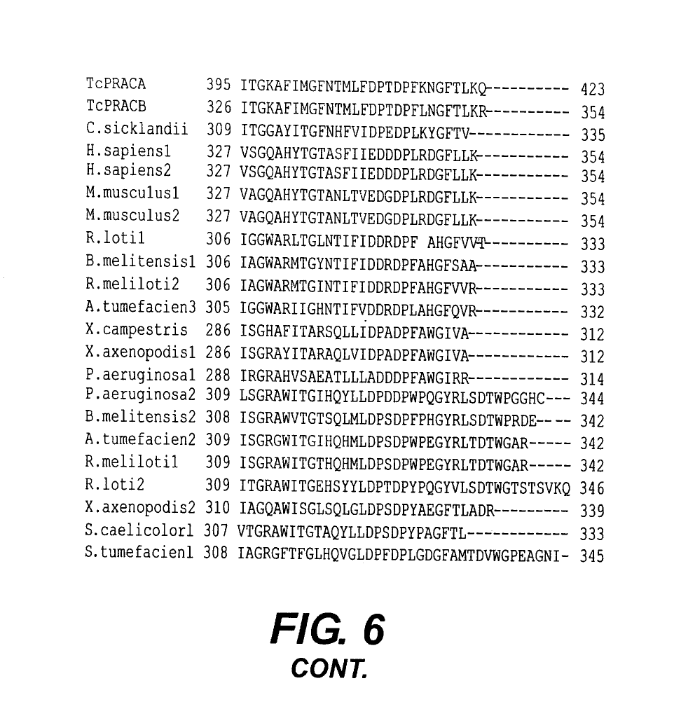

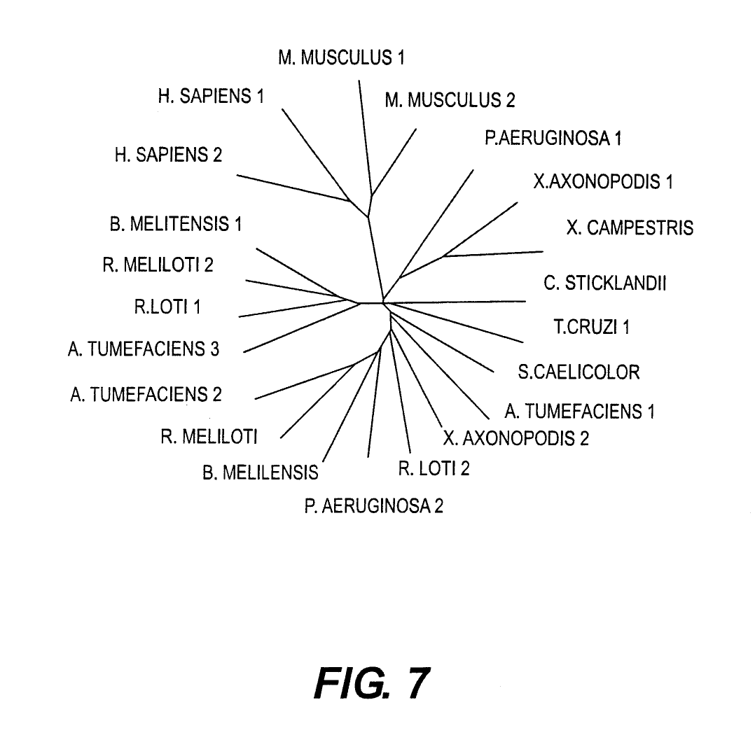

The conservation of critical residues between parasite and bacterial proline racemases prompted a search for similarities between TcPRAC and other protein sequences in SWISS-PROT and TrEMBL databases. Twenty one protein sequences yielded significant homologies, from 11 organisms, such as several proteobacteria of the alpha subdivision (Agrobacterium, Brucella, Rhizobium) and gamma subdivision (Xanthomonas and Pseudomonas), as well as of the fermicutes (Streptomyces and Clostridium). Within the eukaryota, besides in T. cruzi, homologous genes were detected in the human and mouse genomes, where predicted proteins show overall similarities with proline racemase. Except for Clostridium sticklandii and Xantomonas campestri, each other organism encodes 2 paralogues, and Agrobacterium tumefaciens contains 3 genes. The multiple alignment also allowed for the definition of three signatures of proline racemase, which are described here in PROSITE format. As can be seen in Table IV, when using a minimal motif of proline racemase protein (M I), [IVL][GD]XHXXG[ENM]XX[RD]X[VI]XXG, located immediately after the start codon at position 79, the inventors obtained 9 hits. A second motif (M II), consisting of [NSM][VA][EP][AS][FY]X(13,14)[GK]X[IVL]XXD[IV][AS][YWF]GGX[FWY], starting at position 218, gave 14 hits; however, the first or the second half of this motif is not sufficiently stringent to be restrictive for putative proline racemases, but gives hits for different protein families. A third motif (M III), from positions 326 to 339, namely DRSPXGX[GA]XXAXXA, was considered as a minimal pattern. Note that in position 330, the cysteine of the active site was replaced by an X. As shown in Table IV, this minimal pattern yields all 21 hits. Curiously, both genes in human as well as in mouse encode threonine instead of cysteine at the X position in motif III, while in Brucella, Rhizobium and Agrobacterium species each encode one protein with C and one with T in this position. One cannot hypothesize the implications of this substitution for the functionality of these putative proteins. If the residue at position 330 is maintained as a cysteine in motif III, a reduced number of 12 hits from 9 organisms is thus obtained, which can probably be considered as true proline racemases. The alignment of the 21 protein sequences and derived cladogram are shown in FIG. 6 and FIG. 7, respectively, the three boxes depicted correspond to motifs I, II and III described here above. This invention thus shows that DRSPCGXGXXAXXA is the minimal signature for proline racemases. Blast searches against unfinished genomes yielded, at present, an additional 13 predicted protein sequences from 8 organisms, with high similarity to proline racemases, all containing motif III. Organisms are Clostridium difficile, C. botulinum, Bacillus anthracis, Brucella suis, Pseudomonas putida, Rhodobacter sphaeroides, Burkholderia pseudomallei, B. mallei, and the fungus Aspergillus fumigatus. These results indicate that proline racemases might be quite widespread.

TABLE IV
SWISS-PROT and TrEMBL databases
screening using PROSITE motifs

| | | Access. | Motif | | | |
|---|---|---|---|---|---|---|
| Organism | Seq | nb | M I | M II | M III | M III* |
| Agrobacterium tumefaciens | 1 | Q8UIA0 | + | + | + | + |
| Agrobacterium tumefaciens | 2 | Q8U6X2 | − | − | + | − |
| Agrobacterium tumefaciens | 3 | Q8U8Y5 | − | − | + | − |
| Brucella melitensis | 1 | Q8YJ29 | − | + | + | + |
| Brucella melitensis | 2 | Q8YFD6 | + | − | + | − |
| Clostridium stickilandii | | Q9L4Q3 | − | + | + | + |
| Homo sapiens | 1 | Q96EM0 | + | + | + | − |
| Homo sapiens | 2 | Q96LJ5 | + | + | + | − |
| Mus musculus | 1 | Q9CXA2 | + | + | + | − |
| Mus musculus | 2 | Q99KB5 | + | + | + | − |
| Pseudomonas aeruginosa | 1 | Q9I476 | − | + | + | + |
| Pseudomonas aeruginosa | 2 | Q9I489 | − | − | + | + |
| Rhizobium loti | 1 | Q98F20 | − | + | + | + |
| Rhizobium loti | 2 | Q988B5 | + | + | + | − |
| Rhizobium meliloti | 1 | Q92WR9 | − | − | + | − |
| Rhizobium meliloti | 2 | Q92WS1 | − | + | + | + |
| Streptomyces coelicolor | | Q93RX9 | + | − | + | + |
| Trypanosoma cruzi | 1 | Q9NCP4 | + | + | + | + |
| Trypanosoma cruzi | 2 | [symbol] | + | + | + | + |
| Xanthomonas axonopodis | 1 | Q8PJI1 | − | + | + | + |
| Xanthomonas axonopodis | 2 | Q8PKE4 | − | − | + | + |
| Xanthomonas campestris | | Q8P833 | − | + | + | + |
| Bacillus anthracis (Ames) | 1 | Q81UH1 | + | − | + | + |
| Bacillus anthracis (Ames) | 2 | Q81PH1 | − | − | + | + |
| Bacillus cereus | 1 | Q81HB1 | + | − | + | + |
| Bacillus cereus | 2 | Q81CD7 | − | − | + | + |
| Brucella suis | 1 | Q8FYSO | + | + | + | + |
| Brucella suis | 2 | Q8G213 | + | − | + | − |
| Chromobacterium violaceum | | Q7NU77 | + | + | + | + |
| Photorhabdus luminescens | | Q7N4S6 | + | + | + | + |
| Pseudomonas putida | | Q88NF3 | + | + | + | + |
| Rhodopirella baltica | | Q7UWF3 | − | − | + | + |
| Streptomyces avermitilis | | Q82MDO | + | − | + | + |
| Vibrio parahaemolyticus | | Q87Q20 | + | + | + | + |

SWISS-PROT and TrEMBL databases were screened using motifs I to III (M I, M II and M III).
M I corresponds to [IVL][GD]XHXXG[ENM]XX[RD]X[VI]XXG,
M II to of [NSM][VA] [EP][AS][FY]X(13, 14)[GK]X[IVL][XXD[IV][AS][YWF]GGX[FWY]
M III to DRSPXGXGXXAXXA and
M III* to DRSPCGXGXXAXXA.
Access. nb, SWISS-PROT accession number of the sequence;
seq, sequence number according to FIG. 6;
+ and −, presence or absence respectively of hit using the corresponding motif.

Finally, Table V summarizes the genes in which the proline racemase signature has been identified and the sequences including both crucial residues Cys³³⁰ and Cys¹⁶⁰ of the catalytic site are present.

TABLE V
Results of screening using nucleotide or peptide sequence of TcPRACA

| | | | Motifs | | | | | common |
|---|---|---|---|---|---|---|---|---|
| | | | | | | M III* | MCGH | sequence |
| Organism | Accession number | Database | M I | M II | M III | Cys³³⁰ | Cys¹⁶⁰ | EPRGH |
| Aspergillus fumigatus | Af0787f05.p1c | TIGR | + | − | + | − | + | + |
| [symbol] | TIGR 5085 | TIGR | + | + | + | + | ? | + |
| Bacillus anthracis str. Ames | AE017027 | EMBL | + | + | + | + | + | + |
| Bacillus anthracis str. Ames | AE017033 | EMBL | + | + | + | + | + | + |
| (minus strand) | | | | | | | | |
| [symbol] | TIGR 1392 | TIGR | + | + | + | + | + | + |
| Bacillus cereus ATCC14579 | AE017007 | EMBL | + | + | + | + | + | + |
| (minus strand) | | | | | | | | |
| Brucella suis 1330 (minus | AE104469 | EMBL | + | + | + | + | + | + |
| strand) | | | | | | | | |
| [symbol] | TIGR 29461 | TIGR | + | + | + | + | + | + |
| Burkholderia mallei | contig:33162:b_mallei | TIGR | + | + | + | + | + | EPRGSD |
| [symbol] | TIGR 13373 | TIGR | + | ? | + | + | + | EPRGSD |
| [symbol] | SANGER 28450 | Sanger | + | ? | + | + | + | EPRGSD |
| [symbol] | Cbot12g05.q1c | Sanger | ? | + | + | + | + | + |
| [symbol] | SANGER 36826 | Sanger | + | + | + | + | + | + |
| Clostridium difficile | Clostridium difficile 630 | Sanger | ? | + | + | + | + | + |
| [symbol] | SANGER 1496 | Sanger | + | + | + | + | + | + |
| Clostridium sticklandii | CST130879 | EMBL | + | + | + | + | + | + |
| [symbol] | LM16BINcontig2054 | Sanger | ? | + | + | + | + | EPRGND |
| [symbol] | LM16W5b02.q1c | Sanger | ? | + | ? | ? | + | EPRGND |
| Pseudomonas putida KT2440 | AE016778 | EMBL | + | + | + | + | + | EPRGND |
| [symbol] KT2440 | TIGRpputida 13538 | TIGR | + | ? | + | + | + | EPRGND |
| [symbol] | UTHSC 1063 | UTHSC | + | ? | − | − | + | + |
| [symbol] | TbKIX28b06.qlc | Sanger | ? | + | ? | ? | + | + |
| [symbol] | TbKIX28b06.plc | Sanger | ? | + | ? | ? | + | + |
| [symbol] | Tviv655d02 | Sanger | ? | + | + | + | ? | ? |
| [symbol] | Tviv380d6 | Sanger | + | ? | ? | ? | + | + |
| [symbol] | congo208e06 | Sanger | ? | + | + | + | ? | ? |
| [symbol] | AP005077 | EMBL | + | + | + | + | + | + |

Databases were screened using nucleotide or peptide sequences of TcPRACA.
Motifs I to III (M I, M II and M III) were searched.
M I corresponds to [IVL][GD]XHXXG[ENM]XX[RD]X[VI]XXG,
M II to of [NSM][VA] [EP][AS][FY]X(13, 14)[GK]X[IVL]XXD[IV][AS][YWF]GGX[FFWY]
M III to DRSPXGXGXXAXXA and
M III* to DRSPCGXGXXAXXA.
Access. nbs, TIGR, EMBL or SANGER accession numbers of the sequence;
+ and −, presence or absence respectively of the corresponding motif.
Others, extremely conserved regions outside outside the motifs, including NMCGH which contains one of the active site cysteine.
Sequences presented in annexe pages where the conserved regions of 2 Cysteine residues of the active site are squared, are presented in the table in bold with corresponding Accession numbers.

A variety of free D-amino acids can be found in different mammalian tissues in naturally occurring conditions. Some examples include the presence of D-serine in mammalian brain, peripheral and physiological fluids, or else D-asp that can be also detected in endocrine glands, testis, adrenals and pituitary gland. D-pro and D-leu levels are also very high in some brain regions, pineal and pituitary glands. Some reports attribute to D-amino acids a crucial role as neuromodulators (receptor-mediated neurotransmission), as is the case of D-ser, or as regulators of hormonal secretion, oncogeny and differentiation (i.e. D-asp). It is believed that the most probable origin of naturally occurring D-amino acids in mammalian tissues and fluids is the synthesis by direct racemization of free L-enantiomers present in situ. However, a part from the cloning of serine racemase genes from rat brain and human no other amino acid racemases were identified until now in man. Some others report that D-amino acids present in mammalian tissues are derived from nutrition and bacteria.

The increasing number of reports associating the presence of D-amino acids and pathological processes indicate that the alteration of their level in biological samples would be of some diagnostic value as, for instance, the identification of changes in free levels of D-asp and D-Ala in brain regions of individuals presenting Alzheimer. The amounts of D-asp seems to decrease in brain regions bearing neuropathological changes and is paralleled by an increase of O-ala. Overall, total amounts of D-amino acids increase in the brain of individuals presenting memory deficits in Alzheimer, as compared to normal brains, offering new insights towards the development of new simple methods of D-amino acid detection. In the same line, D-ser concentrations in the brain are altered in Parkinson disease and schizophrenia but other findings clearly associate significant higher concentrations of D-amino acids in plasma of patients with renal diseases or else in plasma of elderly people.

Previous results determined that the polyclonal B cell activation by parasite mitogens contributes to the mechanisms leading to parasite evasion and persistence in the mammalian host. It has also been demonstrated that TcPRAC is a potent B cell mitogen released by the infective forms of the parasite. The TcPRAC inhibition by pyrrole carboxylic acid induces a total loss of TcPRAC B cell mitogenic ability.

It has also been shown that the overexpression of TcPRACA and TcPRACB genes by mutant parasites are able to confer to these mutants a better invasion ability of host cells in vitro. This contrasts to the inability of parasites to survive if these TcPRAC genes are inactivated by genetic manipulation. In addition, the immunization of mice with sub-mitogenic doses of TcPRAC, or with appropriate TcPRAC-DNA vector vaccine preparations, was shown to trigger high levels of specific antibody responses directed to TcPRAC and high levels of immunoprotection against an infectious challenge with live Trypanosoma cruzi.

Altogether, these data suggest that TcPRAC enzyme isoforms are essential elements for parasite survival and fate and also support that parasite praline racemase is a good target for both vaccination and chemotherapy. In fact, the addition of pyrrole carboxylic acid at TcPRAC neutralizing doses to non-infected monkey cell cultures do not interfere with cellular growth. Besides, the utilization of a praline racemase inhibitor in humans would be a priori possible since the absence of the two critical active site cysteine residues (Cys 330 and Cys 160) for the PRAC enzyme activity has been observed in the single sequence that displays some peptide homologies with TcPRAC that was identified by blasting the Human Genome available data with the TcPRAC gene sequence.

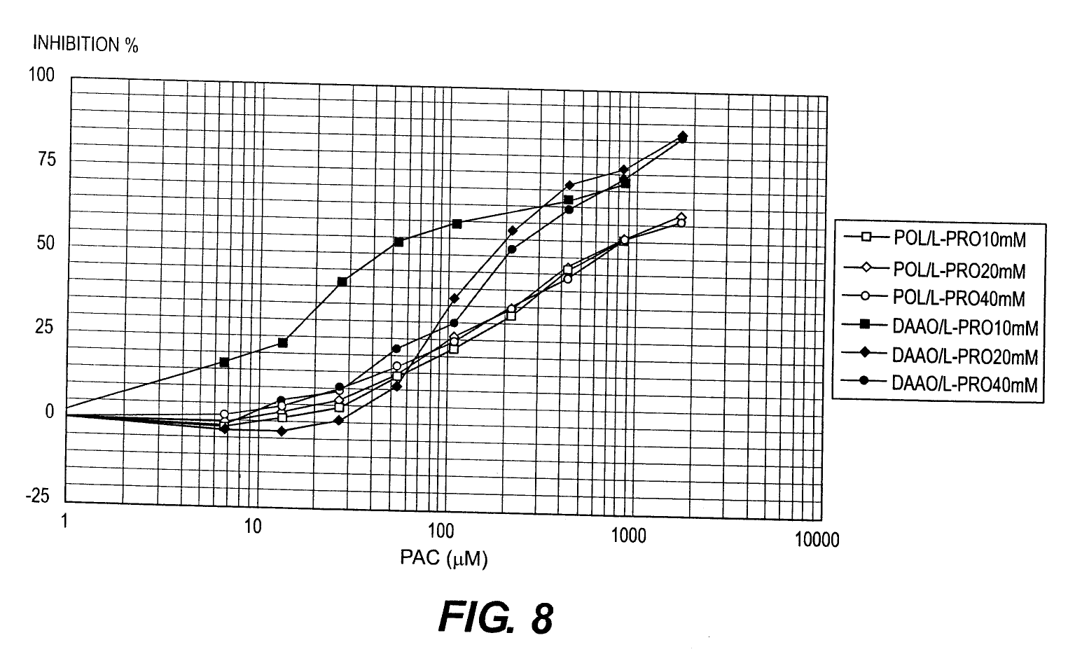

As observed by data mining using TcPRAC gene sequences, it has been possible to identify putative proline racemases in other microrganisms of medical and agricultural interest. As can be seen in FIG. 8, the presence of MI, MII and most particularly MIII stringent motif (the signature for proline racemases) indicates the potentiality of those proteins to be functional proline racemases. On the one hand, it can be observed that critical residues necessary for the enzyme activity are displayed in those sequences and, on the other hand, that the open reading frames (ORF) are highly homologous to the ORF of the parasite PRAC.

In order to search for putative molecules that could be used as inhibitors of TcPRAC, or other proline racemases, it would be necessary to develop a microtest able to specifically reveal the inhibition of proline racemization performed by TcPRAC and consequently the blockage of a given proline stereoisomer generation. For instance, this could be done by analysing the ability of any potential inhibitory molecule to hinder the generation of D-proline in a reaction where L-proline is submitted to TcPRAC enzymatic activity.

At present, the available analyses to detect D- (or L-) amino acids are very challenging and methods to differentiate L-stereoisomers from D-stereoisomers are time-consuming, i.e. gas chromatography, thin layer chromatography using chiral plates, high-performance capillary electrophoretic methods, HPLC, and some enzymatic methods. Some of those techniques also require the use of columns and/or heavy equipment, such as polarimeters or fluorescence detectors.

With the aim of developing a simple test that is useful to rapidly screen putative inhibitors of TcPRAC, TcPRAC constructs allowing for the production of high amounts of the recombinant active enzyme were used together with the knowledge of a specific inhibitor of proline racemases (pyrrole carboxylic acid, PAC) to develop a medium/high throughput microplate test that can be used to easily screen a high number of inhibitor candidates (i.e. 100-1000). Such a test is based on colorimetric reactions that are certainly a simpler alternative to polarimetry and other time-consuming tests. Thus, the evaluation of light deviation of L- or D-proline enantiomers by a polarimeter to quantify the inhibition of proline racemization to test such an elevated number of molecules is impracticable, offers a low sensibility, and would require greater amounts of reagents as compared to a microplate test that would additionally be of an affordable price.

Accordingly, this invention is based on the detection of D-proline originated through racemization of L-proline by TcPRAC, in the presence or in the absence of known concentrations of PAC inhibitor as positive and negative controls of racemization, respectively. For that purpose, this invention utilizes another enzyme, D-amino acid oxidase (D-AAO), that has the ability to specifically oxidize D-amino acids in the presence of a donor/acceptor of electrons and yield hydrogen peroxide. The advantage of this strategy is that hydrogen peroxide can be classically quantified by peroxidase in a very sensitive reaction involving ortho-phenylenediamine, for example, ultimately offering a chromogenic reaction that is visualized by colorimetry at 490 nm.

Since D-amino acid oxidase reacts indiscriminately with any “D-amino acid”, and not with their L-stereoisomers, such a test is not only helpful to identify proline racemase inhibitors, but also applicable, if slightly modified, to detect any alterations in levels of free D-aa in various fluids to make a diagnosis of some pathogenic processes.

### I-Basics for a D-Amino-Acid Quantitative Test

The following method of the invention allows detection and quantitation of D-Amino acids. A first reaction involves a D-amino-oxidase. This enzyme specifically catalyses an oxidative deamination of D-amino-acids, together with a prosthetic group, either Flavin-Adenin-Dinucleotide (FAD) or Flavin-Mononucleotide (FMN), according to the origin of the Enzyme. (Obs. FAD if the enzyme comes from porcine kidney).

The general reaction is as follows:

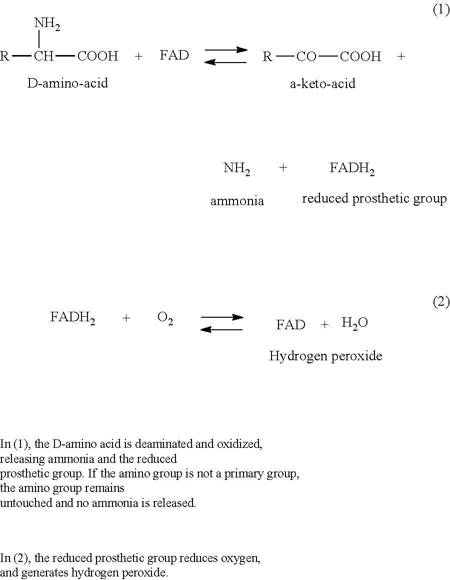

Either a catalase or a peroxidase can decompose hydrogen peroxide.
A catalase activity is written as:

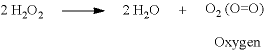

whereas a peroxidase activity is

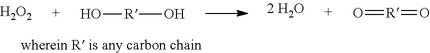

Thus, detection of hydrogen peroxide can be done with the use of catalase and a reagent sensitive to oxygen such as by destaining reduced methylene blue for instance with oxygen or with the use of peroxidase with a change in color of the reagent indicated by:

HO—R′—OH→O═R′═O

II-Application of Such a Test for Evaluating the T. Cruzi Racemase Activity and the Inhibition of this Racemase.

### II-1-Test for Racemase Activity

The T. cruzi racemase activity converts reversibly L-Pro into D-Pro. Since these two forms can induce polarized light deviation, this conversion can be measured by optical polarized light deviation. But the presence of the D-form allows also the use of D-amino-acid oxidase in order to assess the amount of D-Proline in racemase kinetics. In this test the following reactions are involved:

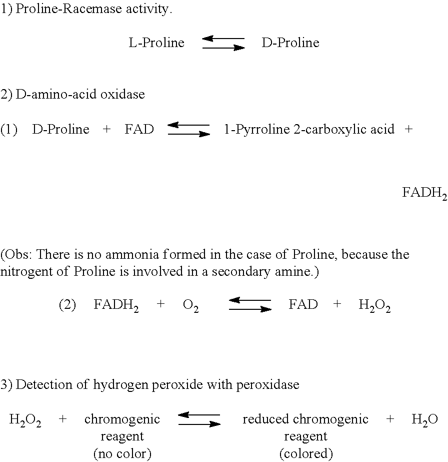

The chromogenic reagent can be, for example, orthophenylenediamine (OPD), or 3,3′,5,5′ tetramethyl benzidine (TMB), or 5-aminosalicylic acid (ASA).

These reactions can be carried out using the following exemplary, but preferred, materials and methods.

### II-1-1—Materials

| Materials | Comments |
|---|---|
| Proline-racemase (TcPRAC) (1 mg/ml Stock) | |
| L-Proline, Sigma, Ref. P-0380 (1M Stock) | An equimolar of D- and L-Proline is made by |
| D-Proline, Aldrich, ref. 85 891-9 (1M Stock) | mixing equal volumes of 2M D-Proline with 2M L-Proline |
| Orthophenylenediamine (OPD) Sigma ref P-8287 | 10 mg tablets. Extemporaneously used as a |
| lot 119H8200 | 20 mg/ml stock solution in water. |
| D-AAO from swine kidney (Sigma) ref. A-5222 lot | Powder dissolved into 1 ml Buffer* + 1 ml 100% |
| 102K1287 | glycerol. The resulting activity is 50 U/ml. Stored at −20° C. |
| Horse radish peroxidase (HRP) Sigma ref P8375 | Powder dissolved into 2.5 ml Buffer* + 2.5 ml 100% |
| lot 69F95002 | glycerol. The resulting activity is 5042 U/ml. Stored at −20° C. |
| Sodium acetate 0.2M Ph 6.0 | |
| Flavine-adenine-dinucleotide (FAD) (Sigma) ref. | Stock solution of 10⁻¹M in water. Stored at −20° C. |
| F-6625 | Used as a 10⁻³M sub-stock solution. |
| Sodium pyrophosphate (Pop) 0.235M | Not soluble at a higher concentration. |
| | Must be stored at 4° C. and gently heated |
| | before use in order to solubilize crystals |
| | which may occur. |
| Buffer* = 10 ml of 0.2M sodium acetate | The final pH is 8.3. |
| buffer pH 6.0 + 680 μl 0.235M Pop | |
| Microplates (96 wells) | With adhesive coverlid |
| ELISA reader for microplates | With a wavelength filter at 490 nm for OPD substrate. |

### II-1-2—Methods

### II-1-2.1—Racemisation in Microplates:

(1) The volumes are indicated for a single well, but duplicates are mandatory. Leave enough raws of the microplate empty for standard and controls to be used in further steps. Distribute the following volumes per well reactions:

a) without inhibitor (Vol=QS 81 μl)

| | | | | |
|---|---|---|---|---|
| TcPRAC 1 mg/ml | 2 μl | 2 μl | 2 μl | 2 μl |
| L-Proline 0.1M | 32 μl | 16 μl | 8 μl | 4 μl |
| Proline Final | (40 mM) | (20 mM) | (10 mM) | (5 mM) |
| concentration | | | | |
| Sodium acetate | 47 μl | 63 μl | 71 μl | 75 μl |
| buffer 0.2M | | | | |
| pH 6 | | | | |

b) with inhibitor (Vol=QS 81 μl)

A range of concentrations between 5 mM and 1 mM can be planned for the inhibitor. It should be diluted in sodium acetate buffer 0.2 M pH 6.0. Hence, the volume of inhibitor is subtracted from the volume of buffer added in order to reach a final volume of 81 μl. For instance, 50% inhibition of racemisation of 10 mM L-proline is obtained with 45 μM Pyrrole carboxylic acid (PAC, specific inhibitor of proline racemase), when 36.5 μl PAC+44.5 μl buffer are used (see results in FIG. 8).

Table VI is provided for 10 mM L-Proline as a substrate.

TABLE IV

| | | | | | | | | | | |
|---|---|---|---|---|---|---|---|---|---|---|
| TcPrac 1 mg/ml | 2 μl | 2 μl | 2 μl | 2 μl | 2 μl | 2 μl | 2 μl | 2 μl | 2 μl | 2 μl |
| L-Proline 0.1M | 8 μl | 8 μl | 8 μl | 8 μl | 8 μl | 8 μl | 8 μl | 8 μl | 8 μl | 8 μl |
| PAC | 0 μl | 5.4 μl | 11 μl | 22 μl | 43 μl | 9 μl | 17μl | 35 μl | 69 μl | 14 μl |
| 0.1 mM/1 mM**/10 mM*** | | | | | | ** | ** | ** | ** | *** |
| Final concentration (μM) | 0 | 6.7 | 13.5 | 27 | 54 | 107 | 214 | 429 | 858 | 1715 |
| Sodium acetate buffer 0.2M | 71 μl | 65.6 μl | 60 μl | 49 μl | 28 μl | 62 μl | 54 μl | 36 μl | 2 μl | 57 μl |
| pH6 QS 81 μl | | | | | | | | | | |

(2) Cover the microplate with an adhesive coverlid and leave for 30 nm at 37° C.
(3) At the end of racemisation, 5.5 μl of 0.235M Pop are added in each reaction well of the microplate in order to shift pH from pH6.0 to pH 8.3.

### II-1-2.1-2—Quantitation of Formed D-Proline: Standards and Controls.

### (1) Prepare Standard and Controls:

Standard: An equimolar mixture of L- and D-Proline is used as a standard in a range from 0.05 mM to 50 mM (final concentration in the assay). It is used for assessing the amount of D-Proline formed after racemization. The standard range is made in microtubes, as follows:

In tube 1, mix Proline and buffer according to the described proportions.

Then, add 500 μl of the obtained mixture to 500 μl of buffer in next tube, and so on.

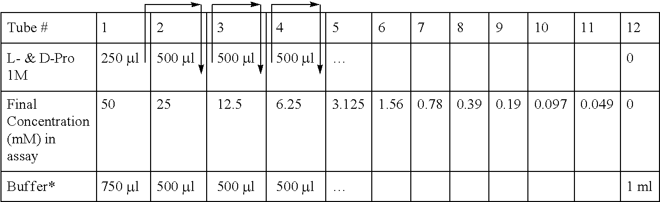

Negative control: is prepared in an other microtube, as follows:

L-Proline (1M) 200 μl

Buffer* 800 μl

Final concentration 40 ml

### Blank=Buffer*.

(2) Dispense in the empty wells of the microplate (see step II-1-2.1):

Buffer* 67 μl

Standard dilutions 20 μl

or negative control

Obs: For the blank dispense 87 μl of Buffer* only

(3) Prepare a mixture containing the enzymes (D-AAO/HRP Mix), as follows:
The amounts are given for one well, provided that the final volume will be 100 μl with the racemase products or the substrate:

| | For 13 μl: |
|---|---|
| Buffer* | 6.5 μl |
| D-AAO 50 U/ml | 1.7 μl |
| OPD (20 mg/ml) | 2.5 μl |
| HRP 5000 U/ml | 0.75 μl |
| FAD 10⁻³M (4.5 μl 10⁻¹M + 446 μl buffer) | 1.5 μl |

This mixture is kept in the ice until use.
(4) The quantitation reaction starts when 13 μl of D-AAO/HRP mix is added to the reaction well.
(5) The microplate is covered with an adhesive coverlid and it is left in the dark at 37° C. between 30 nm and 2 hours. The reaction can be monitored by eye whenever a color gradient matches the D-amino acid concentration of the standard dilutions.
(6) The microplate is read with a microplate spectrophotometer using a filter of at 490 nm.

### Example 13

### D-AOO Microplate Test is More Sensitive than D-Amino Acid Detection by Detection in Polarimeter

In order to compare the D-Proline quantitation by polarimeter and by D-amino-oxidase/HRP a comparison was performed between the two tests using different concentrations of L-proline and different concentrations of PAC, the specific inhibitor of proline racemases. FIG. 8 shows the percent of racemisation inhibition of different L-proline concentrations (ranging from 10-40 mM) using the D-AAO (D-AA0/L-) microtest as compared to conventional detection using a polarimeter (Pol/L-).

With the polarimeter, there seems to be no difference of PAC inhibition of TcPRAC with the three concentrations of L-Proline. Therefore, 50% inhibition is obtained with 1 mM PAC, whether 10 mM or 40 mM L-Proline is used. In contrast, when using D-AAO/HRP test, it can be seen that inhibition by PAC is somewhat higher with a low concentration of L-Proline (10 mM for example) than with an increased one (20 mM or 40 mM). Therefore, 50% inhibition is obtained:
with 50 μM PAC when 10 mM L-Proline is used,
with 170 μM PAC when 20 mM L-Proline is used and
with 220 μM PAC when 40 mM L-Proline is used.

In conclusion, D-AAO/HRP evaluation is more sensitive since it can discriminate PAC inhibition at a lower concentration than evaluation with the polarimeter. Furthermore, inhibition is logically conversely proportional to L-Proline concentration, which can be assessed with the D-AAO/HRP method, but not with the polarimeter measurement. Such a test is useful for the screening of new inhibitors of TcPRAC in a medium/high throughput test.

A preferred technological platform to perform the above test and to select appropriate inhibitors contains at least the following products:

L-Proline, D-Proline, a proline-racemase

A peroxidase, a substrate of a peroxidase

A D-amino-acid oxidase

And optionally a battery of potential inhibitory molecules.

### Example 14

### L-Proline Inhibits D-Amino-Oxidase Activity

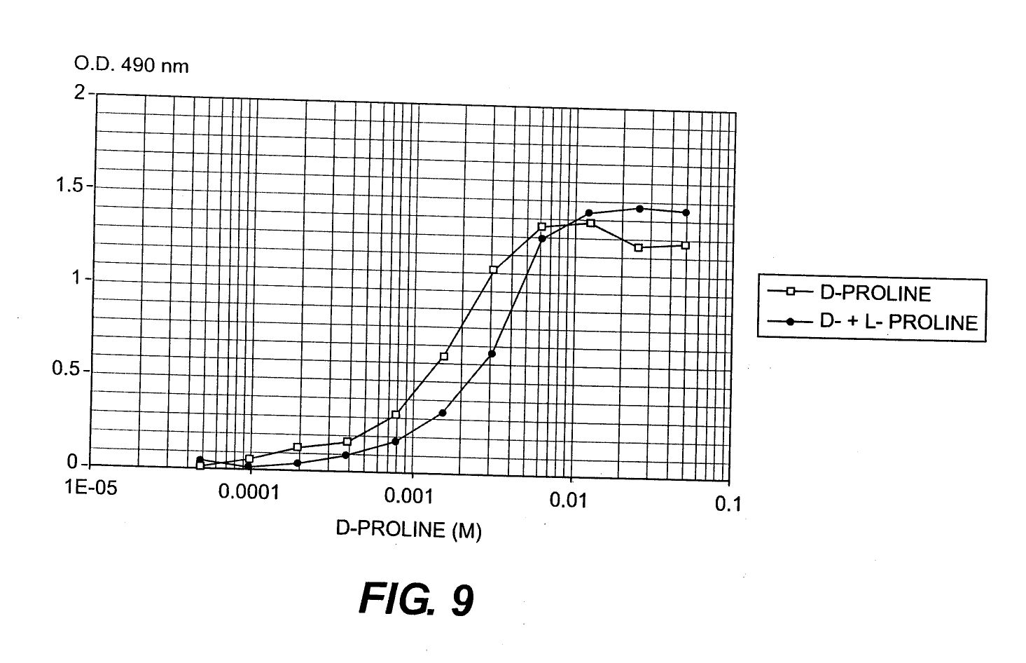

FIG. 9 shows the comparison of D-AAO/HRP reaction using D-Proline alone or an equimolar mixture of D- and L-Proline as standard. It can be seen that the amount of D-Proline required to obtain a given optical density is higher when a mixture of L- and D-Proline are used as compared to a standard using D-proline alone. Since Proline-racemase activity ends when both L- and D-Proline are in equal amounts, it was also adequate to use an equimolar mixture of both enantiomers of Proline as standard for D-Proline determination.

### Example 15

### PAC does not Interfere with DAAO/HRP Activity

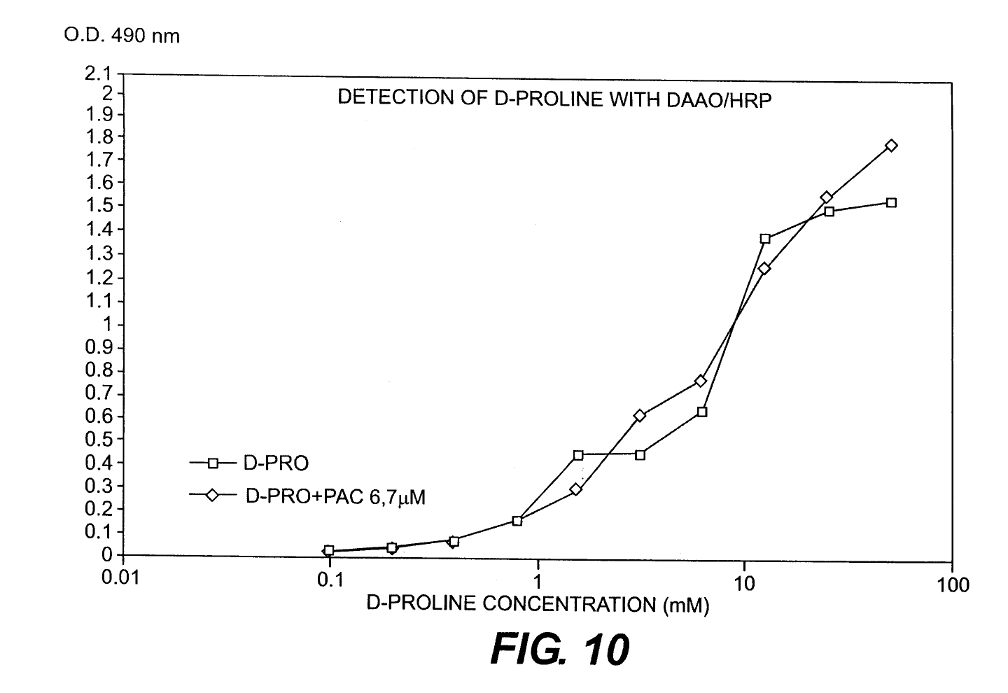

FIG. 10 shows optical density at 490 nm as a function of D-proline concentration under the following conditions.

Conditions in μl wells,

[D-Proline] range between 0.1 mM and 40 mM

[D-AAO] . . . 0.89 μml

[HRP} . . . 37.5 μml

[OPD] . . . 0.5 U/ml

[FAD] . . . 1.5×10⁻⁵ M

Buffer*

The presence of PAC does not influence DAAO/HRP reaction.

### Example 16

### A Medium/High Throughput Test Using the D-AAO Microplate Test

Table VII is an Example of a medium/high throughput test using the D-AAO microplate test.

Blue: D-proline standard (column 1)
Green: Positive control of racemization using avec 10 mM substrate (column 2, line A and B)
Orange: control for inhibition of racemization reaction by PAC using 10 mM substrate (column 2, line C and D)
Blank 1: mix with racemase (column 2, line E)
Blank 2: mix without racemase (column 2, line F)
Yellow: Negative control for specificity of (without racemase+40 mM L-proline) (column 2, line G and I-I)
Other wells: with Inhibitors (T1, T2, T3, . . . T40): in duplicates

TABLE VII

| 1 | | | | | | | | | | | | |
|---|---|---|---|---|---|---|---|---|---|---|---|---|
| D-Pro | | | | | | | | | | | | |
| (mM) | | 2 | 3 | 4 | 5 | 6 | 7 | 8 | 9 | 10 | 11 | 12 |
| A | 10 | L-Pro | T1 | T2 | T3 | T4 | T5 | T6 | T7 | T8 | T9 | T10 |
| B | 5 | L-Pro | ″ | ″ | ″ | ″ | ″ | ″ | ″ | ″ | ″ | ″ |
| C | 2.5 | L-Pro + | T11 | T12 | T13 | T14 | T15 | T16 | T17 | T18 | T19 | T20 |
| | | PAC | | | | | | | | | | |
| D | 1.25 | L-Pro + | ″ | ″ | ″ | ″ | ″ | ″ | ″ | ″ | ″ | ″ |
| | | PAC | | | | | | | | | | |
| E | 0.62 | Blanc 1 | T21 | T22 | T23 | T24 | T25 | T26 | T27 | T28 | T29 | T30 |
| F | 0.31 | Blanc 2 | ″ | ″ | ″ | ″ | ″ | ″ | ″ | ″ | ″ | ″ |
| G | 0.15 | L-Pro | T31 | T32 | T33 | T34 | T35 | T36 | T37 | T38 | T39 | T40 |
| H | 0.07 | L-Pro | ″ | ″ | ″ | ″ | ″ | ″ | ″ | ″ | ″ | ″ |

### Example 17

### Application of Such a Test for General Detection of D-Amino Acids in Samples

The use of a microplate test based on D-amino-acid oxidase together with a peroxidase, such as horseradish peroxidase, can be used to detect and quantitate any D-amino acid in any biological or chemical sample. For example, since D-amino acids are described to be involved in several pathological processes or neurological diseases, such as Alzheimer disease, Parkinson, or renal diseases, their detection can be an important marker or parameter for the diagnosis and the follow-up of these pathologies. This technology can be also extended to the detection and quantification of D-amino acids in eukaryotic organisms, such as plants or fungi, and in bacteria.

The D-AAO/HRP test described here above can also be used for this purpose with slight modifications. For that purpose, the racemase reaction step should be skipped and the microplate test should start straightforward at the 11-1-2.1-2 step described above with the following remarks:

1) Standard: It should not be an equimolar mixture of D- and L-amino acid, but rather a serial dilution of D-Amino acids. The choice of amino acid is made according to the interest of the D-amino acid under investigation. The final volume in wells should be of 87 μl.

2) Negative control: It is made with the L-enantiomer of the D-amino acid under investigation. The final volume should be 87 μl.

3) Blank: It is made with 87 μl buffer*. (See paragraph 11.1.1 Materials.)

4) Samples: The samples to be tested should be adjusted to pH 8.3 with buffer* and their final volumes should be of 87 μl per well.

Obs: Standards, negative controls, samples to test and blanks should be made in duplicates. They are dispensed into the wells of the microplate.

5) Then, the procedure follows steps 3) to 6), as above.

Several D-amino acids and their L-counterparts have been tested using the microplate test described above. Tables VIII and IX show that D-forms of Tyrosine, Valine, Threonine, Glutamic acid, Lysine and Tryptophane are indeed substrates for the D-AAO/HRP and are detected by the test, as described for D-Proline. The results also show that no L-amino acid is detected by such a methodology.

TABLE VIII

| | | | | | | | | | | | | | |
|---|---|---|---|---|---|---|---|---|---|---|---|---|---|
| A | Blank | 49.5 | 24.75 | 12.37 | 6.19 | 3.09 | 1.55 | 0.77 | 0.39 | 0.19 | 0.09 | 0.05 | D-pro |
| B | Blank | 49.5 | 24.75 | 12.37 | 6.19 | 3.09 | 1.55 | 0.77 | 0.39 | 0.19 | 0.09 | 0.05 | mM |
| C | Blank | L-Tyr | L-Val | L-Thr | L-Glu | L-Lys | L-Try | | | | | | |
| D | Blank | 12.5 | 12.5 | 12.5 | 12.5 | 12.5 | 12.5 | mM | | | | | |
| E | Blank | D-Tyr | D-Val | D-Thr | D-Glu | D-Lys | D-Try | | | | | | |
| F | Blank | 6.25 | 6.25 | 6.25 | 6.25 | 6.25 | 6.25 | mM | | | | | |

Optical densities at 490 nm obtained after D-AAO reaction. (raw OD data).

TABLE IX

| | | | | | | | | | | | | | |
|---|---|---|---|---|---|---|---|---|---|---|---|---|---|
| A | 0.105 | 1.961 | 1.757 | 1.814 | 1.983 | 1.716 | 1.234 | 0.809 | 0.496 | 0.308 | 0.213 | 0.173 | D-pro |
| B | 0.118 | 2.004 | 1.885 | 1.976 | 1.949 | 1.879 | 1.221 | 0.824 | 0.504 | 0.32 | 0.215 | 0.159 | mM |
| C | 0.123 | 0.193 | 0.135 | 0.124 | 0.131 | 0.125 | 0.131 | L- | | | | | |
| D | 0.125 | 0.141 | 0.129 | 0.128 | 0.141 | 0.131 | 0.138 | L- | | | | | |
| E | 0.120 | 1.317 | 1.683 | 0.215 | 0.147 | 0.243 | 0.615 | D- | | | | | |
| F | 0.105 | 0.991 | 1.612 | 0.157 | 0.116 | 0.157 | 0.662 | D- | | | | | |

Template of microplate, where, a serial dilution of D-Proline (mM) was made as positive control of the D-AAO reaction. Blank wells containing buffer* are shown. Different L- and D-amino acids were tested, namely Tyrosine (Tyr), Valine (Val), Threonine (Thr), Glutamic acid (Glu), Lysine (Lys) and Tryptophan (Try). To highlight the sensitivity of the D-AAO microtest, higher concentrations of L-enantiomers (12.5 mM) were used in the reactions as compared to the concentrations used for D-enantiomers (6.25 mM):

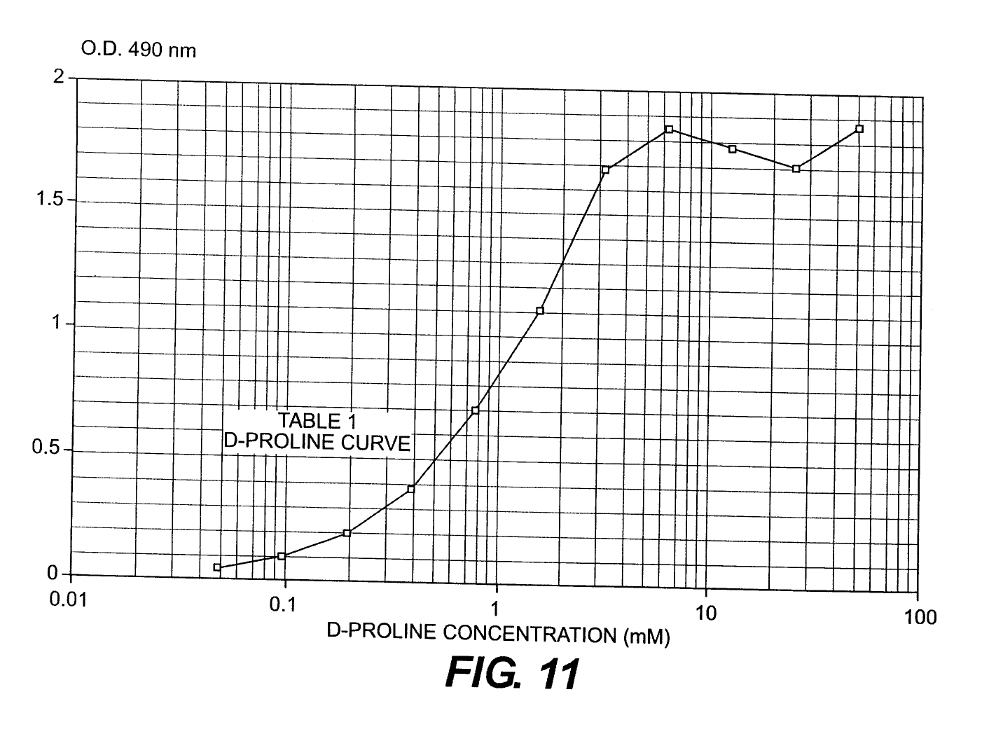

FIG. 11 is a Graph obtained with the serial dilutions of D-proline, as positive reaction control Obs: OD of wells (−) average of OD obtained from blank wells.

A preferred platform to search and quantitate the presence of a D-Amino acid in samples contains at least the following products:

A D-amino acid,

A peroxidase and a substrate of a peroxidase

A D-amino-acid oxidase

And optionally, a L-amino acid enantiomer, as control.

Finally, this invention relates to a method for screening a molecule, which can modulate a racemase activity, wherein the method comprises:
(A) modulating a racemase activity by means of a molecule being tested in the presence of an equimolar mixture of a L- and D-amino acid and of a racemase to be modulated;
(B) oxidatively deaminating the D-amino acid generated in step (A) by means of a D-amino oxidase in a prosthetic group; and
(C) detecting the hydrogen peroxide generated by the oxidative deamination;
   wherein modulation of the hydrogen peroxide is indicative of the capability of the tested molecule to modulate racemase activity. Preferably the molecule inhibits racemase activity, and more preferably the racemase is a proline racemase, for example, Tripanosoma curzi proline racemase. A molecule identified by a method is also part of this invention.

Further, this invention relates to technological platform and all reagents and devices necessary to perform the methods of the invention. The technological platform comprises:
a) L-amino acid, D-amino acid, and a racemase;
b) a peroxydase and a substrate of a peroxydase, or a catalase and a reagent sensitive to oxygen;
c) a D-amino acid oxidase; and
d) optionally, one or more molecules to be screened for inhibitory activity of said racemase.

Preferably, the racemase is a proline racemase and the L-amino acid and D-amino acid are L-proline and D-proline, respectively.

A molecule inhibits a proline racemase containing a subsequence selected from the SEQ ID NO: 1, 2, 3 or 4.

### REFERENCES

The following references are incorporated by reference, in their entirety, herein.
1. Lamzin, V. S., Dauter, Z., and Wilson, K. S. (1995) Curr Opin Struct Biol 5, 830-836.
2. Kleinkauf, H., and von Dohren, H. (1987) Annu. Rev. Microbiol. 41, 259-289
3. Fisher, G. H. (1998) Exs 85, 109-118
4. Nagata, Y., Fujiwara, T., Kawaguchi-Nagata, K., Fukumori, Y., and Yamanaka, T. (1998) Biochim Biophys Acta 1379, 76-82.
5. Nagata, Y., Tanaka, K., Iida, T., Kara, Y., Yamada, R., Nakajima, Y., Fujiwara, T., Fukumori, Y., Yamanaka, T., Koga, Y., Tsuji, S., and Kawaguchi-Nagata, K. (1999) Biochim Biophys Acta 1435, 160-166.
6. Oguri, S., Kumazaki, M., Kitou, R., Nonoyama, H., and Tooda, N. (1999) Biochim Biophys Acta 1472, 107-114.
7. Neidle, A., and Dunlop, D. S. (1990) Life sci. 46, 1512-1522
8. Schell, M. J., Molliver, M. E., and Snyder, S. H. (1995) Proc. Nat. Acad. Sci. 92, 3948-3952
9. Wolosker, H., Blackshaw, S., and Snyder, S. H. (1999) Proc Nat. Acad Sci U S A 96, 13409-13414.
10. Nagata, Y., Homma, H., Matsumoto, M., and Imai, K. (1999) FEBS 454, 317-320
11. Cardinale, G. J., and Abeles, R. H. (1968) Biochemistry 7, 3970-3978
12. Rudnick, G., and Abeles, R. H. (1975) Biochemistry 14, 4515-4522
13. Reina-San-Martin, B., Degrave, W., Rougeot, C., Cosson, A., Chamond, N., Cordeiro-dá-Silva, A., Arala-Chaves, M., and Minoprio, P. (2000) Nature Medicine 6, 890-897
14. Cano, M. I., Gruber, A., Vazquez, M., Cortés, A., Levin, M. J., González, A., Degrave, W., Rondinelli, E., Zingales, B., Ramirez, J. L., Alonso. C., Requena, J. M., and Silveira, J. F. d. (1995) Mol. Bio. Par. 71, 273-278
15. Higuchi, R., Krummel, B., and Saiki, K. K. (1988) Nuc. Ac. Res. 16, 7351-7367
16. Keenan, M. V., and Alworth, W. L. (1974) Biochem Biophys Res Commun 57, 500-504.
17. Fisher, L. M., Albery, W. J., and Knowles, J. R. (1986) Biochemistry 25, 2529-2537.
18. Albery, W. J., and Knowled, J. R. (1986) Biochemistry 25, 2572-2577.
19. Breitbart, R. E., Andreadis, A., and Nadal-Ginard, B. (1987) Annu. Rev. Biochem. 56, 467-495
20. Manning-Cela, R., Gonzalez, A., and Swindle, J. (2002) Infect. Immun. 70, 4726-4728
21. Krassner, S. M., and Flory, B. (1972) J. Protozoa 19, 917-920
22. Bowman, I. B. R., Srivastava, H. K., and Flynn, I. W. (1972) Adaptation in oxidative metabolism during the transformation of Trypanosoma rhodesiense from bloodstream into culture forms, Van den Bossche H. Ed. Comparative Biochemistry of parasites, Academic Press, New York
23. Evans, D. A., and Brown, R. C. (1972) J. Protozool. 19, 686-690
24. Auerswald, L., Schneider, P., and Gade, G. (1998) J. Exp. Biol. 201, 2333-2342
25. Sylvester, D., and Krassner, S. M. (1976) Comp. Biochem. Physiol. 55B, 443-447
26. de Isola, E. L., Lammel, E. M., Katzin, V. J., and Gonzalez Cappa, S. M. (1981) J. Parasitol. 67, 53-58
27. Contreras, V. T., Salles, J. M., Thomas, N., Morel, C. M., and Goldenberg, S. (1985) Mot Biochem. Parasitol. 16, 315-327
28. Silber, A. M., Tonelli, R. R., Martinelli, M., Colli, W., and Alves, M. J. (2002) J. Eukaryot. Microbiol. 49, 441-446
29. Janeway, C. A., and Humphrey, J. H. (1970) Folia Biol. 16, 156-172
30. Mozes, E., Kohn, L. D., Hakim, F., and Singer, D. S. (1993) Science 261, 91-92
31. Sela, M., and Zisman, E. (1997) FASEB J. 11, 449-456
32. Contreras, V. T., Morel, C. M., and Goldenberg, S. (1985) Mol Biochem Parasitol 14, 83-96
33. Souto-Padron, T., Reyes, M. B., Leguizamon, S., Campetella, O. E., Frash, A. C., and de Souza, W. (1989) Eur. J. Cell Biol. 50, 272-278
34. Janes, B. K., and Bender, R. A. (1998) J Bacterial 180, 563-570.
35. de Jong, M. H., van der Drift, C., and Vogels, G. D. (1975) J. Bacterial. 123, 824-827
36. Shakibaei, M., and Frevert, U. (1996) J. Exp. Med. 184, 1699-1711
37. Burleigh, B. A., and Andrews, N. W. (1998) Cur. Op. Microbiol. 1, 461-465
38. Gao, W., Worts, H. H., and Pereira, M. A. (2002) Internat. Immunol. 14, 299-308
39. Martin, D., Ault, B., and Nadler, J. V. (1992) Eur. J. Pharmacol. 219, 59-66
40. Van Harreveld, A. (1980) J. Neurobiol. 11, 519-529
41. Thompson, R. J., Bouwer, H. G., Portnoy, D. A., and Frankel, F. R. (1998) Infect Immun 66, 3552-3561.
42. Wolosker, H., Sheth, K. N., Takahashi, M., Mothet, J. P., Brady, R. O., Jr., Ferris, C. D., and Snyder, S. H. (1999) Proc Natl Acad Sci USA 96, 721-725.
43. Watanabe, T., Shibata, K., Kera, Y., and Yamada, R. (1998) Amino Acids 14, 353-360
¹ GenBank accession number AF195522
² GenBank accession number AY140947
³ EMBL accession number E10199.
⁴ The proline racemase/B-cell mitogen of Trypanosoma cruzi is a virulence factor whose mRNA is differentially regulated through development by alternative splicing. N. Chamond, N. Coatnoan, J. C. Barale, A. Cosson, A. Bememan, W. Degrave and P. Minoprio. Manuscript in preparation.

### Bibliography Related to the Microtest Using D-Amino Acid Oxidase According to the Invention:

1. Reina-San-Martin B., Degrave W., Rougeot C., Cosson A., Chamond N., Cordeiro-da-Silva A., Arala-Chaves M. & Minoprio P. (2000) A B-cell mitogen from a pathogenic trypanosome is a eukaryotic proline racemase. Nature Medicine, 6, 890.
2. Chamond N., Gregoire C., Coatnoan N., Rougeot C., Freitas-Junior L. H., da Silveira J. F., Degrave W. M. & Minoprio P. (2003) Biochemical characterization of proline racemases from the human protozoan parasite Trypanosoma cruzi and definition of putative protein signatures. J Biol Chem, 278, 15484.
3. Chamond N., Coatnoan N. & Minoprio P. (2002) Immunotherapy of Trypanosoma cruzi infections. Current Drug Targets, 2, 247.
4. Rassi A. & Luquetti A. O. (1992) Therapy of Chagas Disease. In: Chagas Disease (American Trypanosomiasis): its impact on transfusion and clinical medicine (ed. S. Wendel, Z. Brener, M. E. Camargo & A. Rassi), p. 237. ISBT Brazil '92-SBHH, Sao Paulo.
5. Cancado J. R. (1999) Criteria of Chagas disease cure. Mem Inst Oswaldo Cruz, 94 Suppl 1, 331.
6. Urbina J. A. (2001) Specific treatment of Chagas disease: current status and new developments. Curr Opin Infect Dis, 14, 733.
7. Urbina J. A. (2002) Chemotherapy of Chagas disease. Curr Pharm Des, 8, 287.
8. Donald, G.& McNeil Jr. (2003) Rare Infection Threatens to Spread in Blood Supply, in New York Times, Nov. 18, 2003
9. Beard, C., Pye, G. Steurer, F. J., Rodriguez, R., Campman, R., Townsend Peterson, A., Ramsey, J., Wirtz, R. A. & Robinson, L. E. Chagas Disease in a Domestic Transmission Cycle, Southern Texas, USA. (2003) Emerging Infectious Diseases, 9, 103.
10. Hamase K., Morikawa A. & Zaitsu K. (2002) D-amino acids in mammals and their diagnostic value. J Chromatogr B Analyt Technol Biomed Life Sci, 781, 73.
11. D'Aniello A., Lee J. M., Petrucelli L. & Di Fiore M. M. (1998) Regional decreases of free D-aspartate levels in Alzheimer's disease. Neurosci Lett, 250, 131.
12. D'Aniello A., Di Fiore M. M., Fisher G. H., Milone A., Seleni A., D'Aniello S., Perna A. F. & Ingrosso D. (2000) Occurrence of D-aspartic acid and N-methyl-D-aspartic acid in rat neuroendocrine tissues and their role in the modulation of luteinizing hormone and growth hormone release. FASEB J, 14, 699.
13. Fisher G. H., D'Aniello A., Vetere A., Padula L., Cusano G. P. & Man E. H. (1991) Free D-aspartate and D-alanine in normal and Alzheimer brain. Brain Res Bull, 26, 983.
14. Fisher G. H., Torres D., Brune J., Cerwinski S., Martin T., Bergljung C., Gruneiro A., Chou S. J., Man E. H. & Pappatheodorou S. (1995) Presence of D-aspartate and D-glutamate in tumor proteins. Cancer Biochem Biophys, 15, 79.
15. Fisher G., Lorenzo N., Abe H., Fujita E., Frey W. H., Emory C., Di Fiore M. M. & A D. A. (1998) Free D- and L-amino acids in ventricular cerebrospinal fluid from Alzheimer and normal subjects. Amino Acids, 15, 263.
16. Fisher G. H. (1998) Appearance of D-amino acids during aging: D-amino acids in tumor proteins. Exs, 85, 109.
17. Nagata Y., Akino T., Ohno K., Kataoka Y., Ueda T., Sakurai T., Shiroshita K. & Yasuda T. (1987) Free D-amino acids in human plasma in relation to senescence and renal diseases. Clin Sci (Colch), 73, 105.
18. Nagata Y., Masui R. & Akino T. (1992) The presence of free D-serine, O-alanine and D-proline in human plasma. Experientia, 48, 986.
19. Chouinard M. L., Gaitan D. & Wood P. L. (1993) Presence of the N-methyl-D-aspartate-associated glycine receptor agonist, D-serine, in human temporal cortex: comparison of normal, Parkinson, and Alzheimer tissues. J Neurachem, 61, 1561.
20. Kumashiro S., Hashimoto A. & Nishikawa T. (1995) Free D-serine in post-mortem brains and spinal cords of individuals with and without neuropsychiatric diseases. Brain Res, 681, 117.
21. Wellner D. and L. A. Lichtenberg, (1968), Assay of Amino acid oxidase, Methods in Enzymology XVII, “metabolism of Amino acids”, 593
22. Scannone H., D. Wellner and A. Novogrodsky, (1964), A study of amino acid oxidase specificity, using a new sensitive assay, Biochemistry, 3, 1742.
23. Kishimoto M. and Takahashi T., (2001), A spectrophotometric microplate Assay for L-amino acid oxidase, Analytical Biochemistry, 298, 136.
24. Wolosker H., Sheth K. N., Takahashi M., Mothet J.-P., Brady R. O. Jr, Ferris, C. D. and Snyder S. H., (1999), Purification of serine racemase: Biosynthesis of the neuromodulator D-Serine, Proc. Nat. Acad. Sci. USA, 96, 721

What is claimed is:

1. A purified nucleic acid molecule encoding a peptide consisting of a motif selected from SEQ ID NOS: 1, 2, 3, or 4.

2. A purified nucleic acid molecule that hybridizes to either strand of a denatured, double-stranded DNA comprising the nucleic acid molecule of claim 1 under conditions of moderate stringency.

3. A recombinant vector that directs the expression of a nucleic acid molecule selected from the group consisting of the purified nucleic acid molecules of claim 1 or 2.

4. A purified polypeptide encoded by a nucleic acid molecule selected from the group consisting of the purified nucleic acid molecules of claims 1 or 2, Motif I (SEQ ID NO:1), Motif II (SEQ ID NO:2), Motif III (SEQ ID NO:3), and Motif III* (SEQ ID NO:4).

5-8. (canceled)

9. Purified antibodies that bind to a polypeptide of claim 4.

10. (canceled)

11. Purified antibodies that bind to a polypeptide of claim 5.

12. (canceled)

13. Purified antibodies that bind to a polypeptide of claim 6.

14. (canceled)

15. Purified antibodies that bind to a polypeptide of claim 7.

16. (canceled)

17. Purified antibodies that bind to a polypeptide of claim 8.

18. (canceled)

19. A host cell transfected or transduced with the recombinant vector of claim 3.

20-22. (canceled)

23. A method of detecting a racemase encoded by a nucleotide sequence containing a subsequence encoding a peptide selected form SEQ ID NO: 1, 2, 3, or 4, said method comprising:
(a) contacting the nucleotide sequence with a primer or a probe, which hybridizes with the nucleic acid molecule of claim 1;
(b) amplifying the nucleotide sequence using said primer or said probe; and
(c) detecting a hybridized complex formed between said primer or probe and the nucleotide sequence.

24. A method of detecting a racemase encoded by a nucleotide sequence containing a subsequence encoding a peptide selected from SEQ ID NO: 1, 2, 3, or 4, said method comprising:
(a) contacting the product encoded by a nucleotide sequence racemase with antibodies according to any one of claims 9 to 18; and
(b) detecting the resulting immunocomplex.

25. A kit for detecting a racemase encoded by a nucleotide sequence containing a subsequence encoding a peptide selected form SEQ ID NO: 1, 2, 3, or 4, said kit comprising:
(a) a polynucleotide primer or probe, which hybridizes with the polynucleotide sequence of claim 1; and
(b) reagents to perform a nucleic acid hybridization reaction.

26. A kit for detecting a racemase encoded by a nucleotide sequence containing a subsequence encoding a peptide selected form SEQ ID NO: 1, 2, 3, or 4, said kit comprising:
(a) purified antibodies according to any one of claim 9 or 10;
(b) standard reagents in a purified form; and
(c) detection reagents.

27. An in vitro method of screening for an active molecule capable of inhibiting a racemase encoded by a nucleotide sequence containing a subsequence encoding a peptide selected form SEQ ID NO: 1, 2, 3, or 4, said method comprising:
(a) contacting the active molecule with said racemase;
(b) testing the capacity of the active molecule, at various concentrations, to inhibit the activity of the racemase; and
(c) choosing the active molecule that provides an inhibitory effect of at least 80% on the activity of the racemase.

28. An immunizing composition containing at least a purified polypeptide according to claim 4, capable of inducing an immune response in vivo, and a pharmaceutical carrier.

29. A method for detecting a D-amino acid, wherein the method comprises:
(A) providing a reaction medium containing the D-amino acid;
(B) reacting the D-amino acid with a D-amino oxidase with a prosthetic group to form a reduced prosthetic group by oxidative deamination of the D-amino acid with a primary amine or oxidation of the D-amino acid with a secondary amine;
(C) reacting the reduced prosthetic group with oxygen to form hydrogen peroxide; and
(D) detecting the hydrogen peroxide thus formed.

30-37. (canceled)

38. A method for detecting racemase activity in a reaction medium, wherein the method comprises:
(A) providing a reaction medium containing a D-amino acid specific to the racemase to be detected;
(B) reacting the D-amino acid with a D-amino oxidase with a prosthetic group to form a reduced prosthetic group by oxidation of the D-amino acid;
(C) reacting the reduced prosthetic group with oxygen to form hydrogen peroxide; and
(D) detecting the hydrogen peroxide thus formed;
wherein the detection of hydrogen peroxide indicates racemase activity tin the reaction medium.

39-41. (canceled)

42. A kit for screening for inhibitors of TcPRAC, wherein the kit comprises:
(A) L-proline, D-proline, and a proline-racemase;
(B) a peroxidase and a substrate of a peroxidase, or a catalase and a reagent sensitive to oxygen;
(C) a D-amino acid oxidase; and
(D) optionally, one or more molecules to be screened for inhibitory activity of TcPRAC.

43. A kit for detecting a D-amino acid in a sample, wherein the kit comprises:
(A) a D-amino acid;
(B) a peroxidase and a substrate of a peroxidase;
(C) a D-amino acid oxidase; and
(D) optionally, a L-amino acid enantiomer as control.

44. A method for detecting a D-amino acid in a sample, wherein the method comprises:
(A) oxidatively deaminating a D-amino acid by reaction with a D-amino acid oxidase in a prosthetic group; and
(B) detecting the hydrogen peroxide generated by the oxidative deamination;
wherein the presence of hydrogen peroxide is indicative of the presence of a D-amino acid in the sample.

45. (canceled)

46. A method for screening a molecule, which can modulate a racemase activity, wherein the method comprises:
(A) modulating a racemase activity by means of a molecule being tested in the presence of an equimolar mixture of a L- and D-amino acid an of a racemase to be modulated;
(B) oxidatively deaminating the D-amino acid generated in step (A) by means of a D-amino oxidase in a prosthetic group; and
(C) detecting the hydrogen peroxide generated by the oxidative deamination;
wherein modulation of the hydrogen peroxide is indicative of the capability of the tested molecule to modulate racemase activity.

47-50. (canceled)

51. A technological platform and all reagents and devices necessary to perform the method of claims 29 to 41 and 44 to 49.

52. The technological platform as claimed in claim 50, comprising
a) L-amino acid, D-amino acid, and a racemase;
b) a peroxydase and a substrate of a peroxydase, or a catalase and a reagent sensitive to oxygen;
c) a D-amino acid oxidase; and
d) optionally, one or more molecules to be screened for inhibitory activity of said racemase.

53. The technological platform as claimed in claim 51, wherein said racemase is a proline racemase and said L-amino acid and D-amino acid are L-proline and D-proline, respectively.

54. A molecule inhibiting a proline racemase containing a subsequence selected form the SEQ ID NO: 1, 2, 3, or 4.

55. A purified nucleic acid molecule encoding a polypeptide consisting of an amino acid sequence that is substantially similar to SEQ ID NO: 1, 2, 3, or 4.

56. The purified nucleic acid molecule of claim 55, wherein the purified nucleic acid molecule hybridizes to either strand of a denatured, double-stranded nucleic acid molecule encoding a polypeptide consisting of the amino acid sequence SEQ ID NO: 1, 2, 3, or 4, wherein the hybridization conditions comprise incubating the nucleic acids at 50° C. in a solution containing 5×SSC and 1×Denhardt's solution and three washes for twenty minutes each in 2×SSC at 60° C.

57. A recombinant vector that directs the expression of the nucleic acid molecule as claimed in claim 55.

58. An isolated host cell transfected or transduced with the recombinant vector as claimed in claim 57.

59. A purified nucleic acid molecule consisting of nucleotides encoding a polypeptides consisting of an amino acid sequence that is substantially similar to SEQ ID NO: 1, 2, 3, or 4.

60. A duplex nucleic acid molecule comprising a single strand of a denatured, double-stranded DNA hybridized to either strand of a denatured, double-stranded DNA comprising the nucleic acid molecule of claim 55 or 61, wherein the hybridization conditions comprise incubating the nucleic acids at 50° C. in a solution containing 5×SSC and 1×Denhardt's solution and three washes for twenty minutes each in 2×SSC at 60° C.

61. The purified nucleic acid molecule of claim 59, wherein the purified nucleic acid molecule hybridizes to either strand of a denatured, double-stranded nucleic acid molecule consisting of nucleotides encoding a polypeptide consisting of the amino acid sequence of SEQ ID NO: 1, 2, 3, or 4, wherein the hybridization conditions comprise incubating the nucleic acids at 50° C. in a solution containing 5×SSC and 1×Denhardt's solution and three washes for twenty minutes each in 2×SSC at 60° C.

62. A recombinant vector that directs the expression of the nucleic acid molecule as claimed in claim 59.

63. An isolated host cell transfected or transduced with the recombinant vector of claim 62.

64. A kit for detecting a racemase encoded by a nucleotide sequence having a subsequence encoding the peptide SEQ ID NO: 1, 2, 3, or 4, said kit comprising:
(a) a polynucleotide primer or probe consisting of the polynucleotide sequence of claim 55 or 61; and
(b) reagents to perform a nucleic acid hybridization reaction.
